(19) United States
(12) Patent Application Publication
(10) Pub. No.: US 2006/0287254 A1
(43) Pub. Date: Dec. 21, 2006

(54) Use of substituted azetidinone compounds for the treatment of sitosterolemia

(75) Inventor: Harry R. Davis JR., Berkeley Heights, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(73) Assignee: Schering Corporation

(21) Appl. No.: 11/437,454

(22) Filed: May 19, 2006

Related U.S. Application Data
(63) Continuation-in-part of application No. 10/057,629, filed on Jan. 25, 2002.
(60) Provisional application No. 60/264,645, filed on Jan. 26, 2001.

(51) Int. Cl. A61K 31/7052; A61K 31/4706; A61K 31/401; A61K 31/397; A61K 31/366; A61K 31/22

(52) U.S. Cl. 514/23; 514/210.02; 514/313; 514/210.03; 514/53; 514/423; 514/460; 514/548

(57) ABSTRACT

The present invention is directed to pharmaceutical compositions comprising a sterol absorption inhibitor, a CEPT inhibitor and/or an HMG-CoA reductase inhibitor as well as methods for treating sitosterolemia, hypercholesterolemia, hyperlipidemia, atherosclerosis, mixed dyslopidemia, vascular events prevention and related disorders in a mammal in need thereof by administering said pharmaceutical compositions to the mammal.

### REFERENCE TO RELATED APPLICATIONS

This application is a continuation-in part of application U.S. Ser. No. 10/057,629, filed Jan. 25, 2002, now pending, which in turn claims the benefit of U.S. Provisional Application Ser. No. 60/264,645 filed Jan. 26, 2001; the disclosure of which are herein incorporated by reference.

### FIELD OF THE INVENTION

The present invention provides methods and pharmaceutical compositions for treating or preventing sitosterolemia, hypercholesterolemia (primary & mixed), hyperlipidemia, atherosclerosis and related diseases by administering to a mammal in need of such treatment an effective amount of at least one treatment composition comprising at least one sterol absorption inhibitor and optionally, an effective amount of at least one bile acid sequestrant or other lipid lowering agent.

### BACKGROUND OF THE INVENTION

Sitosterolemia is a genetic lipid storage disorder, characterized by increased levels of sitosterol and other plant sterols in the plasma and other tissues, due to an increased nonselective intestinal absorption of sterols and decreased hepatic removal. Individuals having sitosterolemia can exhibit tendon and tuberous xanthomas, arthritis, hemolytic eposodes, accelerated atherosclerosis, myocardial infarctions, and die at an early age due to extensive coronary atherosclerosis. See Nguyen et al., “Regulation of cholesterol biosynthesis in sitosterolemia: effects of lovastatin, cholestyramine, and dietary sterol restriction”, Vol 32, Journal of Lipid Research, pp. 1941-1948, (1991).

Sitosterolemia can be treated with bile acid sequestrants (such as cholestyramine, colesevelam hydrochloride and colestipol), however, these compounds have a tendency to cause constipation in patients and therefore compliance with this treatment is difficult. Bile acid sequestrants (insoluble anion exchange resins) bind bile acids in the intestine, interrupting the enterohepatic circulation of bile acids and causing an increase in the fecal excretion of steroids. Use of bile acid sequestrants is desirable because of their non-systemic mode of action. Bile acid sequestrants can lower intrahepatic cholesterol and promote the synthesis of apo B/E (LDL) receptors which bind LDL from plasma to further reduce cholesterol levels in the blood.

Alternative treatments include ileal bypass surgery and selective low density lipoprotein plasmapheresis, which are physically undesirable for the patient.

An improved treatment and related disorders for sitosterolemia is needed which can reduce the concentration of sterols in plasma and tissues, and inhibit associated debilitating physical effects. Such a contribution is provided by this invention.

### SUMMARY OF THE INVENTION

The present invention is directed to a method of treating or preventing sitosterolemia comprising administering to a mammal in need of such treatment an effective amount (i.e. a therapeutically effective amount) of at least one sterol absorption inhibiting compound or a prodrug or a pharmaceutically acceptable salt thereof.

In another embodiment, the present invention is directed to a method of treating or preventing sitosterolemia, comprising administering to a mammal in need of such treatment (i.e. a sitosterolemic mammal), an effective amount of at least one sterol absorption inhibitor in combination with an effective amount of at least one bile acid sequestrant or other lipid lowering agent.

In yet another embodiment, the present invention is directed to a method of treating or preventing sitosterolemia comprising administering to a mammal in need of such treatment an effective amount of at least one sterol absorption inhibiting compound, e.g., substituted azetidinone compounds further described herein below, in combination with at least one bile acid sequestrant such as, for example, cholestyramine, colesevelam hydrochloride and colestipol and/or at least one cholesteryl ester transfer protein (CETP) inhibitor or a prodrug or pharmaceutically acceptable salt thereof.

In still another embodiment, the present invention is directed to a method of treating or preventing sitosterolemia, hyperlipidemia, hypercholesterolemia, mixed dyslipidemia and vascular events prevention comprising administering to a mammal in need of such treatment an effective amount of at least one sterol absorption inhibiting compound, e.g., substituted azetidinone compounds further described herein below, in combination with at least one sterol biosynthesis inhibitor, e.g., a HMG COA Reductase Inhibitor such as, for example, lovastatin, pravastatin, simvastatin, atorvastatin and the like.

Other embodiments of the present invention include pharmaceutical compositions for the treatment or prevention of sitosterolemia, comprising an effective amount of the compositions or combinations used in the methods described above, in a pharmaceutically acceptable carrier.

Another embodiment of this invention is pharmaceutical compositions comprising at least one absorption inhibiting compound or a prodrug or a pharmaceutically acceptable salt thereof and at least one cholesteryl ester transfer protein (CETP) inhibitor or a prodrug or pharmaceutically acceptable salt thereof.

Another embodiment of this invention is the use of pharmaceutical compositions comprising at least one absorption inhibiting compound or a prodrug or a pharmaceutically acceptable salt thereof and at least one CETP inhibitor or a prodrug or pharmaceutically acceptable salt thereof for the treatment or prevention of sitosterolemia, hyperlipidemia, hypercholesterolemia, mixed dyslipidemia and vascular events prevention.

Another embodiment of the present invention is a method of reducing plasma and tissue concentration of at least one sterol selected from the group consisting of phytosterols, 5α-stanols, and mixtures thereof, as well as cholesterol, comprising administering to a mammal in need of such treatment an effective amount of at least one treatment composition comprising at least one sterol absorption inhibitor and optionally at least one CETP inhibitor.

Yet another embodiment of the present invention is a method of reducing plasma and tissue concentration of at least one sterol selected from the group consisting of phytosterols, 5α-stanols and mixtures thereof, as well as cholesterol, comprising administering to a mammal in need of such treatment an effective amount of at least one treatment composition comprising at least one sterol absorption inhibitor and optionally at least one CETP inhibitor.

### DETAILED DESCRIPTION

The present invention provides methods, pharmaceutical compositions and combinations for treating or preventing sitosterolemia. Useful treatment compositions comprise one or more sterol absorption inhibitors represented by Formulas (I-XI) shown below.

In one embodiment one or more sterol absorption inhibitors useful in the methods, compositions or combinations of this invention are represented by Formula (I): 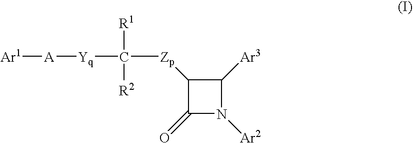
or isomers thereof, or pharmaceutically acceptable salts or solvates of the compounds of Formula (I) or of the isomers thereof, or prodrugs of the compounds of Formula (I) or of the isomers, salts or solvates thereof, wherein:

Ar¹ is R³-substituted aryl;

Ar² is R⁴-substituted aryl;

Ar³ is R⁵-substituted aryl;

Y and Z are independently selected from the group consisting of —CH₂—, —CH(lower alkyl)- and —C(dilower alkyl)-;

A is —O—, —S—, —S(O)— or —S(O)₂—;

R¹ is selected from the group consisting of —OR⁶, —O(CO)R⁶, —O(CO)OR⁹ and —O(CO)NR⁶R⁷; R² is selected from the group consisting of hydrogen, lower alkyl and aryl; or R¹ and R² together are ═O;

q is 1, 2 or 3;

p is 0, 1, 2, 3 or 4;

R⁵ is 1-3 substituents independently selected from the group consisting of —OR⁶, —O(CO)R⁶, —O(CO)OR⁹, —O(CH₂)₁₋₅OR⁹, —O(CO)NR⁶R⁷, —NR⁶R⁷, —NR⁶(CO)R⁷, —NR⁶(CO)OR⁹, —NR⁶(CO)NR⁷R⁸, —NR⁶SO₂-lower alkyl, —NR⁶SO₂-aryl, —CONR⁶R⁷, —COR⁶, —SO₂NR⁶R⁷, S(O)₀₋₂-alkyl, S(O)₀₋₂-aryl, —O(CH₂)₁₋₁₀—COOR⁶, —O(CH₂)₁₋₁₀CONR⁶R⁷, o-halogeno, m-halogeno, o-lower alkyl, m-lower alkyl, -(lower alkylene)-COOR⁶, and —CH═CH—COOR⁶;

R³ and R⁴ are independently 1-3 substituents independently selected from the group consisting of R⁵, hydrogen, p-lower alkyl, aryl, —NO₂, —CF₃ and p-halogeno; R⁶, R⁷ and R⁸ are independently selected from the group consisting of hydrogen, lower alkyl, aryl and aryl-substituted lower alkyl; and

R⁹ is lower alkyl, aryl or aryl-substituted lower alkyl.

Preferred are compounds of formula I wherein Ar¹ is R³-substituted phenyl, especially (4-R³)-substituted phenyl. Ar² is preferably R⁴-substituted phenyl, especially (4-R⁴)-substituted phenyl. Ar³ is preferably R⁵-substituted phenyl, especially (4-R⁵)-substituted phenyl. Mono-substitution of each of Ar¹, Ar² and Ar³ is preferred.

Y and Z are each preferably —CH₂—. R² is preferably hydrogen. R¹ is preferably —OR⁶ wherein R⁶ is hydrogen, or a group readily metabolizable to a hydroxyl (such as —O(CO)R⁶, —O(CO)OR⁹ and —O(CO)NR⁶R⁷, defined above). Also preferred are compounds wherein R¹ and R² together are ═O.

The sum of q and p is preferably 1 or 2, more preferably 1. Preferred are compounds wherein p is zero and q is 1. More preferred are compounds wherein p is zero, q is 1, Y is —CH₂— and R¹ is —OR⁶, especially when R⁶ is hydrogen.

Another group of preferred compounds is that wherein Ar¹ is R³-substituted phenyl, Ar² is R⁴-substituted phenyl and Ar³ is R⁵-substituted phenyl.

Also preferred are compounds wherein Ar¹ is R³-substituted phenyl, Ar² is R⁴-substituted phenyl, Ar³ is R⁵-substituted phenyl, and the sum of p and q is 1 or 2, especially 1. More preferred are compounds wherein Ar¹ is R³-substituted phenyl, Ar² is R⁴-substituted phenyl, Ar³ is R⁵-substituted phenyl, p is zero and q is 1.

A is preferably —O—.

R³ is preferably —COOR⁶, —CONR⁶R⁷, —COR⁶, —SO₂NR⁶R⁷, S(O)₀₋₂-alkyl, S(O)₀₋₂-aryl, NO₂ or halogeno. A more preferred definition for R³ is halogeno, especially fluoro or chloro.

R⁴ is preferably hydrogen, lower alkyl, —OR⁶, —O(CO)R⁶, —O(CO)OR⁹, —O(CO)NR⁶R⁷, —NR⁶R⁷, COR⁶ or halogeno, wherein R⁶ and R⁷ are preferably independently hydrogen or lower alkyl, and R⁹ is preferably lower alkyl. A more preferred definition for R⁴ is hydrogen or halogeno, especially fluoro or chloro.

R⁵ is preferably —OR⁶, —O(CO)R⁶, —O(CO)OR⁹, —O(CO)NR⁶R⁷, —NR⁶R⁷, -(lower alkylene)-COOR⁶ or —CH═CH—COOR⁶, wherein R⁶ and R⁷ are preferably independently hydrogen or lower alkyl, and R⁹ is preferably lower alkyl. A more preferred definition for R⁵ is —OR⁶, -(lower alkylene)-COOR⁶ or —CH═CH—COOR⁶, wherein R⁶ is preferably hydrogen or lower alkyl, is useful in the treatment of sitosterolemia.

In another embodiment, one or more sterol absorption inhibitors useful in the methods, compositions or combinations of this invention are represented by Formula (II): 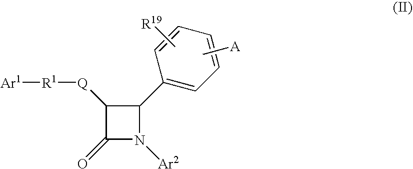
or isomers thereof, or pharmaceutically acceptable salts or solvates of the compounds of Formula (II) or of the isomers thereof, or prodrugs of the compounds of Formula (II) or of the isomers, salts or solvates thereof, wherein:

A is selected from the group consisting of R²-substituted heterocycloalkyl, R²-substituted heteroaryl, R²-substituted benzofused heterocycloalkyl, and R²-substituted benzofused heteroaryl;

Ar¹ is aryl or R³-substituted aryl;

Ar² is aryl or R⁴-substituted aryl;

Q is a bond or, with the 3-position ring carbon of the azetidinone, forms the spiro group 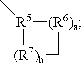
and

R¹ is selected from the group consisting of
—(CH₂)_{q}—, wherein q is 2-6, provided that when Q forms a spiro ring, q can also be zero or 1;
—(CH₂)ₑ-G-(CH₂)ᵣ, wherein G is —O—, —C(O)—, phenylene, —NR⁸— or —S(O)₀₋₂— e is 0-5 and r is 0-5, provided that the sum of e and r is 1-6;
—(C₂-C₆ alkenylene)-; and
—(CH₂)_{f}—V—(CH₂)_{g}—, wherein V is C₃-C₆ cycloalkylene, f is 1-5 and g is 0-5, provided that the sum of f and g is 1-6;

R⁵ is

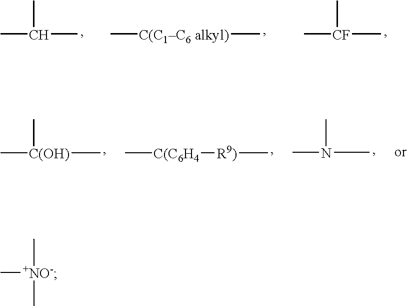

R⁶ and R⁷ are independently selected from the group consisting of —CH₂—, —CH(C₁-C₆ alkyl)-, —C(di-(C₁-C₆) alkyl), —CH═CH— and —C(C₁-C₆ alkyl)=CH—; or R⁵ together with an adjacent R⁶, or R⁵ together with an adjacent R⁷, form a —CH═CH— or a —CH═C(C₁-C₆ alkyl)- group;

a and b are independently 0, 1, 2 or 3, provided both are not zero; provided that when R⁶ is —CH═CH— or —C(C₁-C₆ alkyl)=CH—, a is 1; provided that when R⁷ is —CH═CH— or —C(C₁-C₆ alkyl)=CH—, b is 1; provided that when a is 2 or 3, the R⁶'s can be the same or different; and provided that when b is 2 or 3, the R⁷′s can be the same or different;

and when Q is a bond, R¹ also can be:

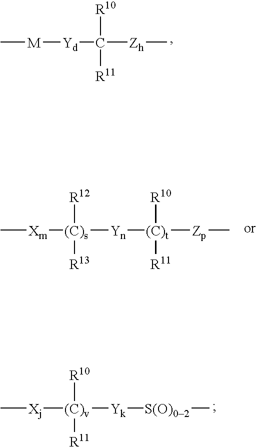

M is —O—, —S—, —S(O)— or —S(O)₂—;

X, Y and Z are independently selected from the group consisting of —CH₂—, —CH(C₁-C₆ alkyl)- and —C(di-(C₁-C₆) alkyl);

R¹⁰ and R¹² are independently selected from the group consisting of —OR¹⁴, —O(CO)R¹⁴, —O(CO)OR¹⁶ and —O(CO)NR¹⁴R¹⁵; R¹¹ and R¹³ are independently selected from the group consisting of hydrogen, (C₁-C₆)alkyl and aryl; or R¹⁰ and R¹¹ together are ═O, or R¹² and R¹³ together are ═O;

d is 1, 2 or 3;

h is 0, 1, 2, 3 or 4;

s is 0 or 1; t is 0 or 1; m, n and p are independently 0-4; provided that at least one of s and t is 1, and the sum of m, n, p, s and t is 1-6; provided that when p is 0 and t is 1, the sum of m, s and n is 1-5; and provided that when p is 0 and s is 1, the sum of m, t and n is 1-5;

v is 0 or 1;

j and k are independently 1-5, provided that the sum of j, k and v is 1-5;

R² is 1-3 substituents on the ring carbon atoms selected from the group consisting of hydrogen, (C₁-C₁₀)alkyl, (C₂-C₁₀)alkenyl, (C₂-C₁₀)alkynyl, (C₃-C₆)cycloalkyl, (C₃-C₆)cycloalkenyl, R¹⁷-substituted aryl, R¹⁷-substituted benzyl, R¹⁷-substituted benzyloxy, R¹⁷-substituted aryloxy, halogeno, —NR¹⁴R¹⁵, NR¹⁴R¹⁵(C₁-C₆ alkylene)-, NR¹⁴R¹⁵C(O)(C₁-C₆ alkylene)-, —NHC(O)R¹⁶, OH, C₁-C₆ alkoxy, —OC(O)R¹⁶, —COR¹⁴, hydroxy(C₁-C₆)alkyl, (C₁-C₆)alkoxy(C₁-C₆)alkyl, NO₂, —S(O)₀₋₂R¹⁶, —SO₂NR¹⁴R¹⁵ and —(C₁-C₆ alkylene)COOR¹⁴; when R² is a substituent on a heterocycloalkyl ring, R² is as defined, or is ═O or 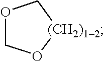
and, where R² is a substituent on a substitutable ring nitrogen, it is hydrogen, (C₁-C₆)alkyl, aryl, (C₁-C₆)alkoxy, aryloxy, (C₁-C₆)alkylcarbonyl, arylcarbonyl, hydroxy, —(CH₂)₁₋₆CONR¹⁸R¹⁸, 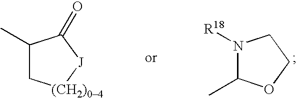
wherein J is —O—, —NH—, —NR¹⁸— or —CH₂—;

R³ and R⁴ are independently selected from the group consisting of 1-3 substituents independently selected from the group consisting of (C₁-C₆)alkyl, —OR¹⁴, —O(CO)R¹⁴, —O(CO)OR¹⁶, —O(CH₂)₁₋₅OR¹⁴, —O(CO)NR¹⁴R¹⁵, —NR¹⁴R¹⁵, —NR¹⁴(CO)R¹⁵, —NR¹⁴(CO)OR¹⁶, —NR¹⁴(CO)NR¹⁵R¹⁹, —NR¹⁴SO₂R¹⁶, —COOR¹⁴, CONR¹⁴R¹⁵, —COR¹⁴, —SO₂NR¹⁴R¹⁵, S(O)₀₋₂R¹⁶, —O(CH₂)₁₋₁₀—COOR¹⁴, —O(CH₂)₁₋₁₀CONR¹⁴R¹⁵, —(C₁-C₆ alkylene)-COOR¹⁴, —CH═CH—COOR¹⁴, —CF₃, —CN, —NO₂ and halogen;

R⁸ is hydrogen, (C₁-C₆)alkyl, aryl (C₁-C₆)alkyl, —C(O)R¹⁴ or —COOR¹⁴;

R⁹ and R¹⁷ are independently 1-3 groups independently selected from the group consisting of hydrogen, (C₁-C₆)alkyl, (C₁-C₆)alkoxy, —COOH, NO₂, —NR¹⁴R¹⁵, OH and halogeno;

R¹⁴ and R¹⁵ are independently selected from the group consisting of hydrogen, (C₁-C₆)alkyl, aryl and aryl-substituted (C₁-C₆)alkyl;

R¹⁶ is (C₁-C₆)alkyl, aryl or R¹⁷-substituted aryl;

R¹⁸ is hydrogen or (C₁-C₆)alkyl; and

R¹⁹ is hydrogen, hydroxy or (C₁-C₆)alkoxy.

As used in Formula (II) above, “A” is preferably an R²-substituted, 6-membered heterocycloalkyl ring containing 1 or 2 nitrogen atoms. Preferred heterocycloalkyl rings are piperidinyl, piperazinyl and morpholinyl groups. The ring “A” is preferably joined to the phenyl ring through a ring nitrogen. Preferred R² substituents are hydrogen and lower alkyl. R¹⁹ is preferably hydrogen.

Ar² is preferably phenyl or R⁴-phenyl, especially (4-R⁴)-substituted phenyl.

Preferred definitions of R⁴ are lower alkoxy, especially methoxy, and halogeno, especially fluoro.

Ar¹ is preferably phenyl or R³-substituted phenyl, especially (4-R³)-substituted phenyl.

There are several preferred definitions for the —R¹-Q- combination of variables:

Q is a bond and R¹ is lower alkylene, preferably propylene;

Q is a spiro group as defined above, wherein preferably R⁶ and R⁷ are each ethylene and R⁵ is

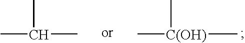

Q is a bond and R¹ is 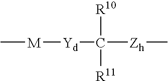
wherein the variables are chosen such that R¹ is —O—CH₂—CH(OH)—;

Q is a bond and R¹ is 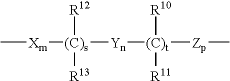
wherein the variables are chosen such that R¹ is —CH(OH)—(CH₂)₂—; and

Q is a bond and R¹ is 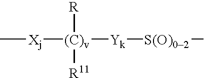
wherein the variables are chosen such that R¹ is —CH(OH)—CH₂—S(O)_{0-2—.}

In another embodiment, one or more sterol absorption inhibitors useful in the methods, compositions or combinations of this invention are represented by Formula (III):

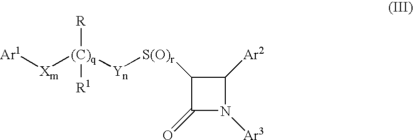

or isomers thereof, or pharmaceutically acceptable salts or solvates of the compounds of Formula (III) or of the isomers thereof, or prodrugs of the compounds of Formula (III) or of the isomers, salts or solvates thereof, wherein:

Ar¹ is aryl, R¹⁰-substituted aryl or heteroaryl;

Ar² is aryl or R⁴-substituted aryl;

Ar³ is aryl or R⁵-substituted aryl;

X and Y are independently selected from the group consisting of —CH₂—, —CH(lower alkyl)- and —C(dilower alkyl)-;

R is —OR⁶, —O(CO)R⁶, —O(CO)OR⁹ or —O(CO)NR⁶R⁷; R¹ is hydrogen, lower alkyl or aryl; or R and R¹ together are ═O;

q is 0 or 1;

r is 0, 1 or 2;

m and n are independently 0, 1, 2, 3, 4 or 5; provided that the sum of m, n and q is 1, 2, 3, 4 or 5;

R⁴ is 1-5 substituents independently selected from the group consisting of lower alkyl, —OR⁶, —O(CO)R⁶, —O(CO)OR⁹, —O(CH₂)₁₋₅OR⁶, —O(CO)NR⁶R⁷—NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, -(lower alkylene)COOR⁶ and —CH═CH—COOR⁶;

R⁵ is 1-5 substituents independently selected from the group consisting of —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, —CF₃, —CN, —NO₂, halogen, -(lower alkylene)COOR⁶ and —CH═CH—COOR⁶;

R⁶, R⁷ and R⁸ are independently selected from the group consisting of hydrogen, lower alkyl, aryl and aryl-substituted lower alkyl;

R⁹ is lower alkyl, aryl or aryl-substituted lower alkyl; and

R¹⁰ is 1-5 substituents independently selected from the group consisting of lower alkyl, —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, —CF₃, —CN, —NO₂ and halogen.

Within the scope of Formula III, there are two preferred structures. In Formula IIIA, q is zero and the remaining variables are as defined above, and in Formula IIIB, q is 1 and the remaining variables are as defined above:

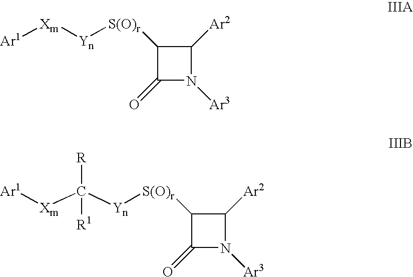

R⁴, R⁵ and R¹⁰ are each preferably 1-3 independently selected substituents as set forth above. Preferred are compounds of Formula (III) wherein Ar¹ is phenyl, R¹⁰-substituted phenyl or thienyl, especially (4-R¹⁰)-substituted phenyl or thienyl. Ar² is preferably R⁴-substituted phenyl, especially (4-R⁴)-substituted phenyl. Ar³ is preferably phenyl or R⁵-substituted phenyl, especially (4-R⁵)-substituted phenyl. When Ar¹ is R¹⁰-substituted phenyl, R¹⁰ is preferably halogeno, especially fluoro. When Ar² is R⁴-substituted phenyl, R⁴ is preferably —OR⁶, especially wherein R⁶ is hydrogen or lower alkyl. When Ar³ is R⁵-substituted phenyl, R⁵ is preferably halogeno, especially fluoro. Especially preferred are compounds of formula III wherein Ar¹ is phenyl, 4-fluorophenyl or thienyl, Ar² is 4-(alkoxy or hydroxy)phenyl, and Ar³ is phenyl or 4-fluorophenyl.

X and Y are each preferably —CH₂—. The sum of m, n and q is preferably 2, 3 or 4, more preferably 2. When q is 1, n is preferably 1 to 5.

Preferences for X, Y, Ar¹, Ar² and Ar³ are the same in each of formulae IIIA and IIIB.

In compounds of formula IIIA, the sum of m and n is preferably 2, 3 or 4, more preferably 2. Also preferred are compounds wherein the sum of m and n is 2, and r is 0 or 1.

In compounds of formula IIIB, the sum of m and n is preferably 1, 2 or 3, more preferably 1. Especially preferred are compounds wherein m is zero and n is 1. R¹ is preferably hydrogen and R is preferably —OR⁶ wherein R⁶ is hydrogen, or a group readily metabolizable to a hydroxyl (such as —O(CO)R⁶, —O(CO)OR⁹ and —O(CO)NR⁶R⁷, defined above), or R and R¹ together form a ═O group.

In another embodiment, one or more sterol absorption inhibitors useful in the methods, compositions or combinations of this invention are represented by Formula (IV): 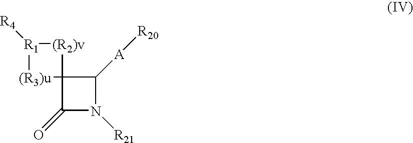
or isomers thereof, or pharmaceutically acceptable salts or solvates of the compounds of Formula (IV) or of the isomers thereof, or prodrugs of the compounds of Formula (IV) or of the isomers, salts or solvates thereof, wherein:
R₁ is 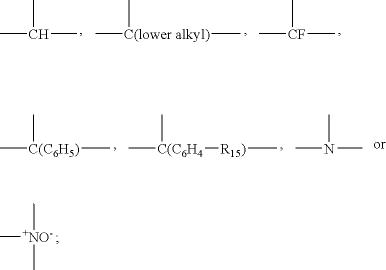
R₂ and R₃ are independently selected from the group consisting of
   —CH₂—,
   —CH(lower alkyl)-,
   —C(di-lower alkyl)-,
   —CH═CH— and
   —C(lower alkyl)=CH—; or
R₁ together with an adjacent R₂, or R₁ together with an adjacent R₃, form a —CH═CH— or a —CH═C(lower alkyl)- group;
u and v are independently 0, 1, 2 or 3,
   provided both are not zero;
   provided that when R₂ is —CH═CH— or —C(lower alkyl)=CH—, v is 1;
   provided that when R₃ is —CH═CH— or —C(lower alkyl)=CH—, u is 1;
   provided that when v is 2 or 3, the R₂'s can be the same or different;
   and provided that when u is 2 or 3, the R₃'s can be the same or different;
R₄ is B—(CH₂)ₘC(O)—, wherein m is 0, 1, 2, 3, 4 or 5;
   B—(CH₂)_{q}—, wherein q is 0, 1, 2, 3, 4, 5 or 6;
   B—(CH₂)ₑ-Z-(CH₂)ᵣ, wherein Z is —O—, —C(O)—, phenylene, —N(R₈)— or —S(O)₀₋₂—, e is 0, 1, 2, 3, 4 or 5 and r is 0, 1, 2, 3, 4 or 5, provided that the sum of e and r is 0, 1, 2, 3, 4, 5 or 6;
   B—(C₂-C₆ alkenylene)-;
   B—(C₄-C₆ alkadienylene)-;
   B—(CH₂)ₜ-Z-(C₂-C₆ alkenylene)-, wherein Z is as defined above, and wherein t is 0, 1, 2 or 3, provided that the sum of t and the number of carbon atoms in the alkenylene chain is 2, 3, 4, 5 or 6;
   B—(CH₂)ₜ—V—(CH₂)_{g}—, wherein V is C₃-C₆ cycloalkylene, f is 1, 2, 3, 4 or 5 and g is 0, 1, 2, 3, 4 or 5, provided that the sum off and g is 1, 2, 3, 4, 5 or 6;
   B—(CH₂)ₜ—V—(C₂-C₆ alkenylene)- or
   B—(C₂-C₆ alkenylene)-V—(CH₂)ₜ—, wherein V and t are as defined above, provided that the sum of t and the number of carbon atoms in the alkenylene chain is 2, 3, 4, 5 or 6;
   B—(CH₂)ₐ-Z-(CH₂)_{b}—V—(CH₂)_{d}—, wherein Z and V are as defined above and a, b and d are independently 0, 1, 2, 3, 4, 5 or 6, provided that the sum of a, b and d is 0, 1, 2, 3, 4, 5 or 6; or
T-(CH₂)ₛ—, wherein T is cycloalkyl of 3-6 carbon atoms and s is 0, 1, 2, 3, 4, 5 or 6; or
R₁ and R₄ together form the group 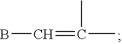
B is indanyl, indenyl, naphthyl, tetrahydronaphthyl, heteroaryl or W-substituted heteroaryl, wherein heteroaryl is selected from the group consisting of:
   pyrrolyl,
   pyridinyl,
   pyrimidinyl,
   pyrazinyl,
   triazinyl,
   imidazolyl,
   thiazolyl,
   pyrazolyl,
   thienyl,
   oxazolyl and
   furanyl, and for nitrogen-containing heteroaryls, the N-oxides thereof, or 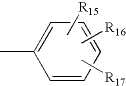
W is 1 to 3 substituents independently selected irom the group consisting of
   lower alkyl,
   hydroxy lower alkyl,
   lower alkoxy,
   alkoxyalkyl,
   alkoxyalkoxy,
   alkoxycarbonylalkoxy,
   (lower alkoxyimino)-lower alkyl,
   lower alkanedioyl,
   lower alkyl lower alkanedioyl,
   allyloxy,
   —CF₃,
   —OCF₃,
   benzyl,
   R₇-benzyl,
   benzyloxy,
   R₇-benzyloxy,
   phenoxy,
   R₇-phenoxy,
   dioxolanyl,
   NO₂,
   —N(R₈)(R₉),
   N(R₈)(R₉)-lower alkylene-,
   N(R₈)(R₉)-lower alkylenyloxy-,
   OH,
   halogeno,
   —CN,
   —N₃,
   —NHC(O)OR₁₀,
   —NHC(O)R₁₀,
   R₁₁O₂SNH—,
   (R₁₁O₂S)₂N—,
   —S(O)₂NH₂,
   —S(O)₀₋₂R₈,
   tert-butyldimethyl-silyloxymethyl,
   —C(O)R₁₂,
   —COOR₁₉,
   —CON(R₈)(R₉),
   —CH═CHC(O)R₁₂,
   -lower alkylene-C(O)R₁₂,
   R₁₀C(O)(lower alkylenyloxy)-,
   N(R₈)(R₉)C(O)(lower alkylenyloxy)- and 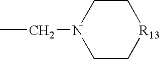
   for substitution on ring carbon atoms,
   and the substituents on the substituted heteroaryl ring nitrogen atoms,
when present, are selected from the group consisting of
   lower alkyl,
   lower alkoxy,
   —C(O)OR₁₀,
   —C(O)R₁₀,
   OH,
   N(R₈)(R₉)-lower alkylene-,
   N(R₈)(R₉)-lower alkylenyloxy-,
   —S(O)₂NH₂ and
   2-(trimethylsilyl)-ethoxymethyl;
R₇ is 1-3 groups independently selected from the group consisting of
   lower alkyl,
   lower alkoxy,
   —COOH,
   NO₂,
   —N(R₈)(R₉),
   OH, and
   halogeno;
R₈ and R₉ are independently H or lower alkyl;
R₁₀ is lower alkyl, phenyl, R₇-phenyl, benzyl or R₇-benzyl;
R₁₁ is OH, lower alkyl, phenyl, benzyl, R₇-phenyl or R₇-benzyl;
R₁₂ is H, OH, alkoxy, phenoxy, benzyloxy, 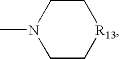
—N(R₈)(R₉), lower alkyl, phenyl or R₇-phenyl;
R₁₃ is —O—, —CH₂—, —NH—, —N(lower alkyl)- or —NC(O)R₁₉;
R₁₅, R₁₆ and R₁₇ are independently selected from the group consisting of H and the groups defined for W; or R₁₅ is hydrogen and R₁₆ and R₁₇, together with adjacent carbon atoms to which they are attached, form a dioxolanyl ring;
R₁₉ is H, lower alkyl, phenyl or phenyl lower alkyl; and
R₂₀ and R₂₁ are independently selected from the group consisting of phenyl, W-substituted phenyl, naphthyl, W-substituted naphthyl, indanyl, indenyl, tetrahydronaphthyl, benzodioxolyl, heteroaryl, W-substituted heteroaryl, benzofused heteroaryl, W-substituted benzofused heteroaryl and cyclopropyl, wherein heteroaryl is as defined above.

One group of preferred compounds of formula IV is that wherein R₂₁ is
phenyl,
W-substituted phenyl,
indanyl,
benzofuranyl,
benzodioxolyl,
tetrahydronaphthyl,
pyridyl,
pyrazinyl,
pyrimidinyl,
quinolyl or
cyclopropyl,

wherein W is
lower alkyl,
lower alkoxy,
OH,
halogeno,
—N(R₈)(R₉),
—NHC(O)OR₁₀,
—NHC(O)R₁₀.
NO₂,
—CN,
—N₃,
—SH,
—S(O)₀₋₂-(lower alkyl),
—COOR₁₉,
—CON(R₈)(R₉),
—COR₁₂,
phenoxy,
benzyloxy,
—OCF₃,
—CH═C(O)R₁₂ or
tert-butyldimethylsilyloxy, wherein R₈, R₉, R₁₀, R₁₂ and R₁₉ are as defined for Formula IV. When W is 2 or 3 substituents, the substituents can be the same or different.

Another group of preferred compounds of Formula IV is that wherein R₂₀ is phenyl or W-substituted phenyl, wherein preferred meanings of W are as defined above for preferred definitions of R₂₁.

More preferred are compounds of Formula IV wherein R₂₀ is phenyl or W-substituted phenyl and R₂₁ is
phenyl,
W-substituted phenyl,
indanyl,
benzofuranyl,
benzodioxolyl,
tetrahydronaphthyl,
pyridyl,
pyrazinyl,
pyrimidinyl,
quinolyl or
cyclopropyl;

W is lower alkyl,
lower alkoxy,
OH,
halogeno,
—N(R₈)(R₉),
—NHC(O)OR₁₀,
—NHC(O)R₁₀.
NO₂,
—CN,
—N₃,
—SH,
—S(O)₀₋₂-(lower alkyl),
—COOR₁₉,
—CON(R₈)(R₉),
—COR₁₂,
phenoxy,
benzyloxy,
—CH═CHC(O)R₁₂,
—OCF₃ or
tert-butyl-dimethyl-silyloxy, wherein when W is 2 or 3 substituents, the substituents can be the same or different, and wherein R₈, R₉, R₁₀, R₁₂ and R₁₉ are as defined in Formula IV.

Also preferred are compounds of Formula IV wherein R₁ is

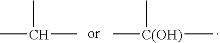

Another group of preferred compounds of Formula IV is that wherein R₂ and R₃ are each —CH₂— and the sum of u and v is 2, 3 or 4, with u=v=2 being more preferred.

R₄ is preferably B—(CH₂)_{q}— or B—(CH₂)ₑ-Z-(CH₂)ᵣ—, wherein B, Z, q, e and r are as defined above. B is preferably 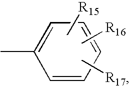
wherein R₁₆ and R₁₇ are each hydrogen and wherein R₁₅ is preferably H, OH, lower alkoxy, especially methoxy, or halogeno, especially chloro.

A preferred definition of Z is —O—, e is preferably 0, and r is preferably 0.

A preferred definition of q is 0-2.

R₂₀ is preferably phenyl or W-substituted phenyl.

Preferred W substituents for R₂₀ are lower alkoxy, especially methoxy and ethoxy, OH, and —C(O)R₁₂, wherein R₁₂ is preferably lower alkoxy.

Preferred definitions for R₂₁ are phenyl, lower alkoxy-substituted phenyl and F-phenyl.

Especially preferred are compounds of Formula IV wherein R₁ is 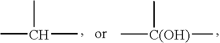
R₂ and R₃ are each —CH₂—, u=v=2, R₄ is B—(CH₂)_{q}—, wherein B is phenyl or phenyl substituted by lower alkoxy or chloro, q is 0-2, R₂₀ is phenyl, OH-phenyl, lower alkoxy-substituted phenyl or lower alkoxycarbonyl-substituted phenyl, and R₂₁ is phenyl, lower alkoxy-substituted phenyl or F-phenyl.

In another embodiment, one or more sterol absorption inhibitors useful in the methods, compositions or combinations of this invention are represented by Formulas (VA) and (VB): 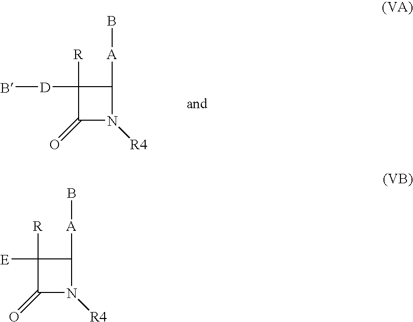
or isomers thereof, or pharmaceutically acceptable salts or solvates of the compounds of Formulas (VA) and (VB) or of the isomers thereof, or prodrugs of the compounds of Formulas (VA) and (VB) or o isomers, salts or solvates thereof, wherein:

A is —CH═CH—, —CδC——(CH₂)ₚ— wherein p is 0, 1 or 2;

B is

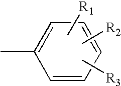

B′ is

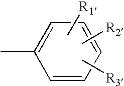

D is —(CH₂)ₘC(O)— or —(CH₂)_{q}— wherein m is 1, 2, 3 or 4 and q is 2, 3 or 4;

E is C₁₀ to C₂₋₀ alkyl or —C(O)-(C₉ to C₁₉)-alkyl, wherein the alkyl is straight or branched, saturated or containing one or more double bonds;

R is hydrogen, C₁-C₁₅ alkyl, straight or branched, saturated or containing one or more double bonds, or B—(CH₂)ᵣ-, wherein r is 0, 1, 2, or 3;

R₁, R₂, R₃, R_{1′}, R_{2′}, and R_{3′} are independently selected from the group consisting of hydrogen, lower alkyl, lower alkoxy, carboxy, NO₂, NH₂, OH, halogeno, lower alkylamino, dilower alkylamino, —NHC(O)OR₅, R₆O₂SNH— and —S(O)₂NH₂;

R₄ is 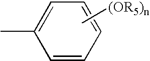
wherein n is 0, 1, 2 or 3;

R₅ is lower alkyl; and

R₆ is OH, lower alkyl, phenyl, benzyl or substituted phenyl wherein the substituents are 1-3 groups independently selected from the group consisting of lower alkyl, lower alkoxy, carboxy, NO₂, NH₂, OH, halogeno, lower alkylamino and dilower alkylamino.

Preferred are compounds of Formula (VA) wherein R is hydrogen, saturated or mono-unsaturated C₁₋C₁₀ alkyl or phenyl. Another group of preferred compounds of Formula (VA) is that wherein D is propyl (i.e., —(CH₂)_{q}— and q is 3). A third group of preferred compounds of Formula (VA) is that wherein R₄ is p-methoxyphenyl or 2,4,6-trimethoxyphenyl. Still another group of preferred compounds of Formula (VA) is that wherein A is ethylene or a bond (i.e., —(CH₂)ₚ— wherein p is zero). R_{1′}, R_{2′}, and R_{3′} are preferably each hydrogen, and preferably R₁ is hydrogen, hydroxy, nitro, lower alkoxy, amino or t-butoxycarbonyl-amino and R₂ and R₃ are each hydrogen.

Especially preferred are compounds of Formula (VA) wherein R_{1′}, R_{2′}, and R_{3′} are each hydrogen; R₁ is hydrogen, hydroxy, nitro, lower alkoxy, amino or t-butoxycarbonyl-amino and R₂ and R₃ are each hydrogen; R is hydrogen, ethyl or phenyl; D is propyl; R₄ is p-methoxyphenyl or 2,4,6-trimethoxyphenyl; and A is ethylene or a bond.

Especially preferred compounds of Formula (VA), wherein B′ is phenyl, are shown in the following table:

| D | R | A | B | R₄ |
|---|---|---|---|---|
| —(CH₂)₃— | H | — | p-MeO- | p-MeO-phenyl |
| | | | phenyl | |
| —CH₂C(O)— | phenyl | — | phenyl | p-MeO-phenyl |
| —(CH₂)₃— | H | — | phenyl | p-MeO-phenyl |
| —(CH₂)₃— | H | — | p-OH- | p-MeO-phenyl |
| | | | phenyl | |
| —(CH₂)₃— | H | ethylene | p-MeO- | p-MeO-phenyl |
| | | | phenyl | |
| —(CH₂)₃— | H | — | 3-MeO- | p-MeO-phenyl |
| | | | phenyl | |
| —(CH₂)₃— | ethyl | — | phenyl | p-MeO-phenyl |
| —(CH₂)₃— | phenyl | — | phenyl | p-MeO-phenyl |
| —(CH₂)₃— | ethyl | — | phenyl | 2,4,6-tri-MeO- |
| | | | | phenyl |
| —(CH₂)₃— | methyl | — | phenyl | p-MeO-phenyl |
| —(CH₂)₃— | H | — | p-NH₂- | p-MeO-phenyl |
| | | | phenyl | |

The first-listed compound in the above table having the (3R,4S) absolute stereochemistry is more preferred.

Preferred compounds of Formula (VB) are those wherein R is hydrogen, methyl, ethyl, phenyl or phenylpropyl. Another group of preferred compounds of Formula (VB) is that wherein R₄ is p-methoxyphenyl or 2,4,6-trimethoxyphenyl. Still another group of preferred compounds of Formula (VB) is that wherein A is ethylene or a bond. Yet another group of preferred compounds of Formula (VB) is that wherein E is decyl, oleoyl or 7-Z-hexadecenyl. Preferably R₁, R₂ and R₃ are each hydrogen.

Especially preferred compounds of Formula (VB) are those wherein R is hydrogen, methyl, ethyl, phenyl or phenylpropyl; R₄ is p-methoxyphenyl or 2,4,6-trimethoxyphenyl; A is ethylene or a bond; E is decyl, oleoyl or 7-Z-hexadecenyl; and R₁, R₂ and R₃ are each hydrogen.

An especially preferred compound of Formula (VB) is that wherein E is decyl, R is hydrogen, B-A is phenyl and R₄ is p-methoxyphenyl.

In another embodiment, one or more sterol absorption inhibitors useful in the methods, compositions or combinations of this invention are represented by Formula (VI): 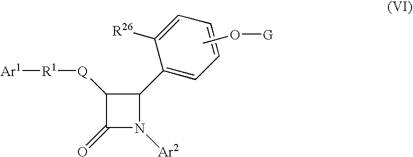
or isomers thereof, or pharmaceutically acceptable salts or solvates of the compounds of Formula (VI) or of the isomers thereof, or prodrugs of the compounds of Formula (VI) or of the isomers, salts or solvates thereof, wherein:

R²⁶ is H or OG¹;

G and G¹ are independently selected from the group consisting of
H, 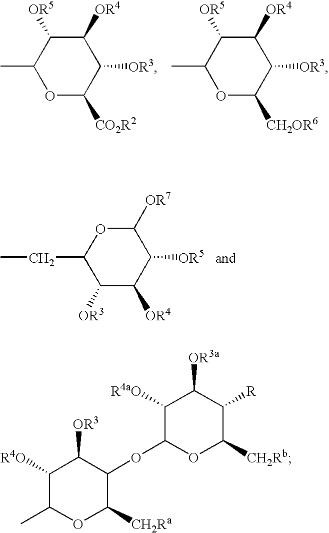
provided that when R²⁶ is H or
OH, G is not H;

R, R^{a} and R^{b} are independently selected from the group consisting of H, —OH, halogeno, —NH₂, azido, (C₁-C₆)alkoxy(C₁-C₆)-alkoxy or —W—R³⁰;

W is independently selected from the group consisting of —NH—C(O)—, —O—C(O)—, —O—C(O)—N(R³¹)—, —NH—C(O)—N(R³¹)— and —O—C(S)—N(R³¹)—;

R² and R⁶ are independently selected from the group consisting of H, (C₁-C₆)alkyl, aryl and aryl(C₁-C₆)alkyl;

R³, R⁴, R⁵, R⁷, R^{3a} and R^{4a} are independently selected from the group consisting of H, (C₁-C₆)alkyl, aryl(C₁-C₆)alkyl, —C(O)(C₁-C₆)alkyl and —C(O)aryl;

R³⁰ is selected from the group consisting of R³²-substituted T, R³²-substituted-T-(C₁-C₆)alkyl, R³²-substituted-(C₂-C₄)alkenyl, R³²-substituted-(C₁-C₆)alkyl, R³²-substituted-(C₃-C₇)cycloalkyl and R³²-substituted-(C₃-C₇)cycloalkyl(C₁-C₆)alkyl;

R³¹ is selected from the group consisting of H and (C₁-C₄)alkyl;

T is selected from the group consisting of phenyl, furyl, thienyl, pyrrolyl, oxazolyl, isoxazolyl, thiazolyl, iosthiazolyl, benzothiazolyl, thiadiazolyl, pyrazolyl, imidazolyl and pyridyl;

R³² is independently selected from 1-3 substituents independently selected from the group consisting of halogeno, (C₁-C₄)alkyl, —OH, phenoxy, —CF₃, —NO₂, (C₁-C₄)alkoxy, methylenedioxy, oxo, (C₁-C₄)alkylsulfanyl, (C₁-C₄)alkylsulfinyl, (C₁-C₄)alkylsulfonyl, —N(CH₃)₂, —C(O)—NH(C₁-C₄)alkyl, —C(O)—N((C₁-C₄)alkyl)₂, —C(O)—(C₁-C₄)alkyl, —C(O)—(C₁-C₄)alkoxy and pyrrolidinylcarbonyl; or R³² is a covalent bond and R³¹, the nitrogen to which it is attached and R³² form a pyrrolidinyl, piperidinyl, N-methyl-piperazinyl, indolinyl or morpholinyl group, or a (C₁-C₄)alkoxycarbonyl-substituted pyrrolidinyl, piperidinyl, N-methylpiperazinyl, indolinyl or morpholinyl group;

Ar¹ is aryl or R¹⁰-substituted aryl;

Ar² is aryl or R₁₁-substituted aryl;

Q is a bond or, with the 3-position ring carbon of the azetidinone,
forms the spiro group 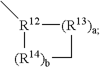
and

R¹ is selected from the group consisting of
a—(CH₂)_{q}—, wherein q is 2-6, provided that when Q forms a spiro ring, q can also be zero or 1;
—(CH₂)ₑ-E-(CH₂)ᵣ—, wherein E is —O—, —C(O)—, phenylene, —NR²²— or —S(O)₀₋₂—, e is 0-5 and r is 0-5, provided that the sum of e and r is 1-6;
—(C₂-C₆)alkenylene-; and
—(CH₂)_{f}—V—(CH₂)_{g}—, wherein V is C₃-C₆ cycloalkylene, f is 1-5 and g is 0-5, provided that the sum of f and g is 1-6;

R¹² is

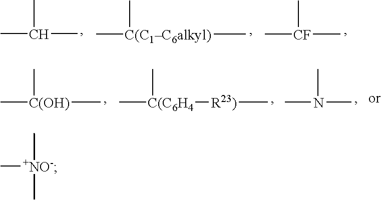

R¹³ and R¹⁴ are independently selected from the group consisting of
—CH₂—,
—CH(C₁-C₆ alkyl)-,
—C(di-(C₁-C₆) alkyl),
—CH═CH— and
—C(C₁-C₆ alkyl)=CH—; or R¹² together with an adjacent R¹³, or R¹² together with an adjacent R¹⁴, form a —CH═CH— or a —CH═C(C₁-C₆ alkyl)-group;

a and b are independently 0, 1, 2 or 3, provided both are not zero;
provided that when R¹³ is —CH═CH— or —C(C₁-C₆ alkyl)=CH—, a is 1;
provided that when R¹⁴ is —CH═CH— or —C(C₁-C₆ alkyl)=CH—, b is 1;
provided that when a is 2 or 3, the R¹³′s can be the same or different;

and provided that when b is 2 or 3, the R¹⁴′s can be the same or different;

and when Q is a bond, R¹ also can be:

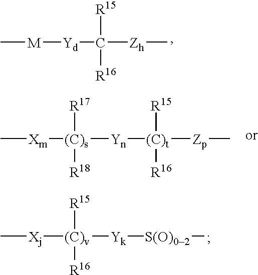

M is —O—, —S—, —S(O)— or —S(O)₂—;

X, Y and Z are independently selected from the group consisting of —CH₂—, —CH(C₁-C₆)alkyl- and —C(di-(C₁-C₆)alkyl);

R¹⁰ and R¹¹ are independently selected from the group consisting of 1-3 substituents independently selected from the group consisting of
(C₁-C₆)alkyl,
—OR¹⁹,
—O(CO)R¹⁹,
—O(CO)OR²¹,
—O(CH₂)₁₋₅OR¹⁹,
—O(CO)NR¹⁹R²⁰,
—NR¹⁹R²⁰,
—NR¹⁹(CO)R²⁰,
—NR¹⁹(CO)OR²¹,
—NR¹⁹(CO)NR²OR²⁵,
—NR¹⁹SO₂R²¹,
—COOR¹⁹,
—CONR¹⁹R²⁰,
—COR¹⁹,
—SO₂NR¹⁹R²⁰,
S(O)₀₋₂R²¹,
—O(CH₂)₁₋₁₀—COOR¹⁹,
—O(CH₂)₁₋₁₀CONR¹⁹R²⁰,
—(C₁-C₆ alkylene)-COOR¹⁹,
—CH═CH—COOR¹⁹,
—CF₃, —CN, —NO₂ and halogen;

R¹⁵ and R¹⁷ are independently selected from the group consisting of —OR¹⁹, —O(CO)R¹⁹, —O(CO)OR²¹ and —O(CO)NR¹⁹R₂₀;

R¹⁶ and R¹⁸ are independently selected from the group consisting of H, (C₁-C₆)alkyl and aryl; or R¹⁵ and R¹⁶ together are ═O, or R¹⁷ and R¹⁸ together are ═O;

d is 1, 2 or 3;

h is 0, 1, 2, 3 or 4;

s is 0 or 1; t is 0 or 1; m, n and p are independently 0-4;
provided that at least one of s and t is 1, and the sum of m, n, p, s and t is 1-6;
provided that when p is 0 and t is 1, the sum of m, s and n is 1-5; and
provided that when p is 0 and s is 1, the sum of m, t and n is 1-5;

v is 0 or 1;

j and k are independently 1-5, provided that the sum of j, k and v is 1-5;

and when Q is a bond and R¹ is 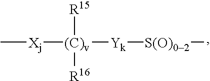
Ar¹ can also be pyridyl, isoxazolyl, furanyl, pyrrolyl, thienyl, imidazolyl, pyrazolyl, thiazolyl, pyrazinyl, pyrimidinyl or pyridazinyl;

R¹⁹ and R²⁰ are independently selected from the group consisting of H, (C₁-C₆)alkyl, aryl and aryl-substituted (C₁-C₆)alkyl;

R²¹ is (C₁-C₆)alkyl, aryl or R²⁴-substituted aryl;

R²² is H, (C₁-C₆)alkyl, aryl (C₁-C₆)alkyl, —C(O)R¹⁹ or —COOR¹⁹;

R²³ and R²⁴ are independently 1-3 groups independently selected from the group consisting of H, (C₁-C₆)alkyl, (C₁-C₆)alkoxy, —COOH, NO₂, —NR¹⁹R²⁰, —OH and halogeno; and

R²⁵ is H, —OH or (C₁-C₆)alkoxy.

Ar² is preferably phenyl or R₁₁-phenyl, especially (4-R¹¹)-substituted phenyl. Preferred definitions of R¹¹ are lower alkoxy, especially methoxy, and halogeno, especially fluoro.

Ar¹ is preferably phenyl or R¹⁰-substituted phenyl, especially (4-R¹⁰)— substituted phenyl. A preferred definition of R¹⁰ is halogeno, especially fluoro.

There are several preferred definitions for the —R¹—O— combination of variables:

Q is a bond and R¹ is lower alkylene, preferably propylene;

Q is a spiro group as defined above, wherein preferably R¹³ and R¹⁴ are each ethylene and R¹² is 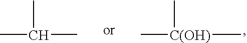
and R¹ is —(CH₂)_{q} wherein q is 0-6;

Q is a bond and R¹ is 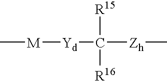
wherein the variables are chosen such that R¹ is —O—CH₂—CH(OH)—;

Q is a bond and R₁ 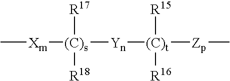
wherein the is
variables are chosen such that R¹ is —CH(OH)—(CH₂)₂—; and

Q is a bond and R¹ is 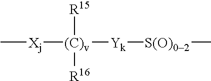
wherein the variables are chosen such that R¹ is —CH(OH)—CH₂—S(O)_{0-2—.}

A preferred compound of Formula (VI) therefore, is one wherein G and G¹ are as defined above and in which the remaining variables have the following definitions:

Ar¹ is phenyl or R¹⁰-substituted phenyl, wherein R¹⁰ is halogeno;

Ar² is phenyl or R¹¹-phenyl, wherein R¹¹ is 1 to 3 substituents independently selected from the group consisting of C₁-C₆ alkoxy and halogeno;

Q is a bond and R¹ is lower alkylene; Q, with the 3-position
ring carbon of the azetidinone, forms the group 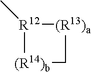
wherein preferably R¹³ and R¹⁴ are each ethylene and a and b are each 1, and wherein R¹² is 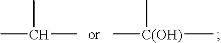
Q is a bond and R¹ is —O—CH₂—CH(OH)—; Q is a bond and R¹ is —CH(OH)—(CH₂)₂—; or Q is a bond and R¹ is —CH(OH)—CH₂—S(O)_{0-2—.}

Preferred variables for G and G¹ groups of the formulae 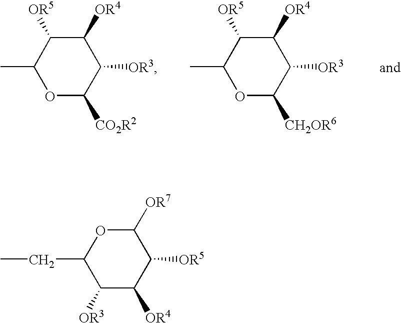
are as follows:

R², R³, R⁴, R⁵, R⁶ and R⁷ are independently selected from the group consisting of H, (C₁-C₆)alkyl, benzyl and acetyl.

Preferred variables for group G or G¹ of the formula 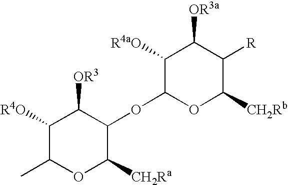
are as follows:

R³, R^{3a}, R⁴ and R^{4a} are selected from the group consisting of H, (C₁-C₆)alkyl, benzyl and acetyl;

R, R^{a} and R^{b} are independently selected from the group consisting of
H,
—OH,
halogeno,
—NH₂,
azido,
(C₁-C₆)alkoxy(C₁-C₆)alkoxy and
—W—R³⁰,

wherein W is —O—C(O)— or —O—C(O)—NR³¹—, R³¹ is H and

R³⁰ is (C₁-C₆)alkyl, —C(O)—(C₁-C₄)alkoxy-(C₁-C₆)alkyl, T, T-(C₁-C₆)alkyl, or T or T-(C₁-C₆)alkyl wherein T is substituted by one or two halogeno or (C₁-C₆)alkyl groups.

Preferred R³⁰ substituents are selected from the group consisting of
2-fluorophenyl,
2,4-difluoro-phenyl,
2,6-dichlorophenyl,
2-methylphenyl,
2-thienylmethyl,
2-methoxy-carbonylethyl,
thiazol-2-yl-methyl,
2-furyl,
2-methoxycarbonylbutyl and
phenyl.

Preferred combinations of R, R^{a} and R^{b} are as follows:

1) R, R^{a} and R^{b} are independently —OH or —O—C(O)—NH—R³⁰, especially wherein R^{a} is —OH and R and R^{b} are —O—C(O)—NH—R³⁰ and R³⁰ is selected from the preferred substituents identified above, or wherein R and R^{a} are each —OH and R^{b} is —O—C(O)—NH—R³⁰ wherein R³⁰ is 2-fluorophenyl, 2,4-difluoro-phenyl, 2,6-dichlorophenyl;

2) R^{a} is —OH, halogeno, azido or (C₁-C₆)-alkoxy(C₁-C₆)alkoxy, R^{b} is H, halogeno, azido or (C₁-C₆)alkoxy(C₁-C₆)-alkoxy, and R is —O—C(O)—NH—R³⁰, especially compounds wherein R^{a} is —OH, R^{b} is H and R³⁰ is 2-fluorophenyl;

3) R, R^{a} and R^{b} are independently —OH or —O—C(O)—R³⁰ and R³⁰ is (C₁-C₆)alkyl, T, or T substituted by one or two halogeno or (C₁-C₆)alkyl groups, especially compounds wherein R is —OH and R^{a} and R^{b} are —O—C(O)—R³⁰ wherein R³⁰ is 2-furyl; and

4) R, R^{a} and R^{b} are independently —OH or halogeno. Three additional classes of preferred compounds are those wherein the C¹ anomeric oxy is beta, wherein the C² anomeric oxy is beta, and wherein the R group is alpha. G and G¹ are preferably selected from: 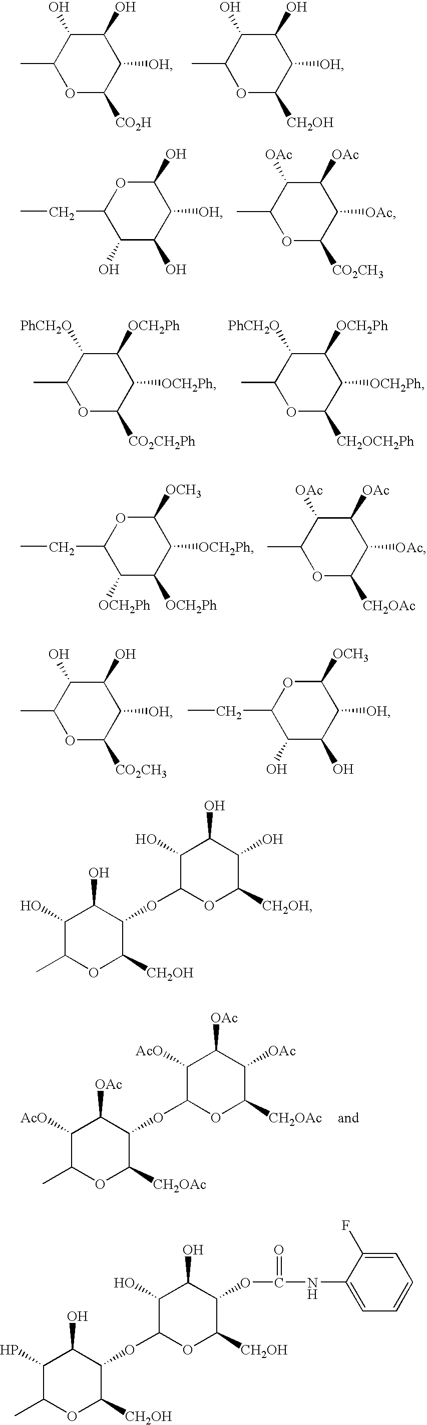
wherein Ac is acetyl and Ph is phenyl.

Preferably, R²⁶ is H or OH, more preferably H. The —O-G substituent is preferably in the 4-position of the phenyl ring to which it is attached.

In another embodiment, one or more sterol absorption inhibitors useful in the methods, compositions or combinations of this invention are represented by Formula (VII): 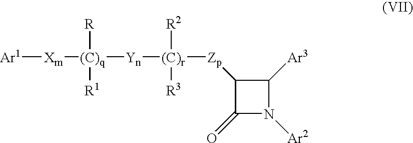
or isomers thereof, or pharmaceutically acceptable salts or solvates of the compounds of Formula (VII) or of the isomers thereof, or prodrugs of the compounds of Formula (VII) or of the isomers, salts or solvates thereof, wherein:

Ar¹ and Ar² are independently selected from the group consisting of aryl and R⁴-substituted aryl;

Ar³ is aryl or R⁵-substituted aryl;

X, Y and Z are independently selected from the group consisting of —CH₂—, —CH(lower alkyl)- and —C(dilower alkyl)-;

R and R² are independently selected from the group consisting of —OR⁶, —O(CO)R⁶, —O(CO)OR⁹ and —O(CO)NR⁶R⁷;

R¹ and R³ are independently selected from the group consisting of hydrogen, lower alkyl and aryl;

q is 0 or 1; r is 0 or 1; m, n and p are independently 0, 1, 2, 3 or 4; provided that at least one of q and r is 1, and the sum of m, n, p, q and r is 1, 2, 3, 4, 5 or 6; and provided that when p is 0 and r is 1, the sum of m, q and n is 1, 2, 3, 4 or 5;

R⁴ is 1-5 substituents independently selected from the group consisting of
lower alkyl,
—O(CO)R⁶,
—O(CO)OR⁹,
—O(CH₂)₁₋₅OR⁶,
—O(CO)NR⁶R⁷,
—NR⁶R⁷,
—NR⁶(CO)R⁷,
—NR⁶(CO)OR⁹,
—NR⁶(CO)NR⁷R⁸
—NR⁶SO₂R⁹,
—COOR⁶,
—CONR⁶R⁷,
—COR⁶,
—SO₂NR⁶R⁷,
S(O)₀₋₂R⁹,
—O(CH₂)₁₋₁₀—COOR⁶,
—O(CH₂)₁₋₁₀CONR⁶R⁷,
-(lower alkylene)COOR⁶,
—CH═CH—COOR⁶,
—CF₃,
—CN,
—NO₂ and
halogen;

R⁵ is 1-5 substituents independently selected from the group consisting of
—OR⁶,
—O(CO)R⁶,
—O(CO)OR⁹,
—O(CH₂)₁₋₅OR⁶,
—O(CO)NR⁶R⁷,
—NR⁶R⁷,
—NR⁶(CO)R⁷,
—NR⁶(CO)OR⁹,
—NR⁶(CO)NR⁷R⁸,
—NR⁶SO₂R⁹,
—COOR⁶,
—CONR⁶R⁷,
—COR⁶,
—SO₂NR⁶R⁷,
S(O)₀₋₂R⁹,
—O(CH₂)₁₋₁₀—COOR⁶,
—O(CH₂)₁₋₁₀CONR⁶R⁷,
-(lower alkylene)COOR⁶ and
—CH═CH—COOR⁶;

R⁶, R⁷ and R⁸ are independently selected from the group consisting of hydrogen, lower alkyl, aryl and aryl-substituted lower alkyl; and

R⁹ is lower alkyl, aryl or aryl-substituted lower alkyl.

R⁴ is preferably 1-3 independently selected substituents, and R⁵ is preferably 1-3 independently selected substituents.

Preferred compounds of Formula (VII) are those in which Ar¹ is phenyl or R⁴-substituted phenyl, especially (4-R⁴)-substituted phenyl. Ar² is preferably phenyl or R⁴-substituted phenyl, especially (4-R⁴)-substituted phenyl. Ar³ is preferably R⁵-substituted phenyl, especially (4-R⁵)-substituted phenyl. When Ar¹ is (4-R⁴)-substituted phenyl, R⁴ is preferably a halogen. When Ar² and Ar³ are R⁴- and R⁵-substituted phenyl, respectively, R⁴ is preferably halogen or —OR⁶ and R⁵ is preferably —OR⁶, wherein R⁶ is lower alkyl or hydrogen. Especially preferred are compounds wherein each of Ar¹ and Ar² is 4-fluorophenyl and Ar³ is 4-hydroxyphenyl or 4-methoxyphenyl.

X, Y and Z are each preferably —CH₂—. R¹ and R³ are each preferably hydrogen. R and R² are preferably —OR⁶ wherein R⁶ is hydrogen, or a group readily metabolizable to a hydroxyl (such as —O(CO)R⁶, —O(CO)OR⁹ and —O(CO)NR⁶R⁷, defined above).

The sum of m, n, p, q and r is preferably 2, 3 or 4, more preferably 3. Preferred are compounds wherein m, n and r are each zero, q is 1 and p is 2.

Also preferred are compounds of Formula (VII) wherein p, q and n are each zero, r is 1 and m is 2 or 3. More preferred are compounds wherein m, n and r are each zero, q is 1, p is 2, Z is —CH₂— and R is —OR⁶, especially when R⁶ is hydrogen.

Also more preferred are compounds of Formula (VII) wherein p, q and n are each zero, r is 1, m is 2, X is —CH₂— and R² is —OR⁶, especially when R⁶ is hydrogen.

Another group of preferred compounds of Formula (VII) are those wherein,

Ar¹ is phenyl or R⁴-substituted phenyl,

Ar² is phenyl or R⁴-substituted phenyl and

Ar³ is R⁵-substituted phenyl.

Also preferred are compounds wherein

Ar¹ is phenyl or R⁴-substituted phenyl,

Ar² is phenyl or R⁴-substituted phenyl,

Ar³ is R⁵-substituted phenyl, and the sum of m, n, p, q and r is 2, 3 or 4, more especially 3.

More preferred are compounds wherein

Ar¹ is phenyl or R⁴-substituted phenyl,

Ar² is phenyl or R⁴-substituted phenyl,

Ar³ is R⁵-substituted phenyl, and wherein m, n and r are each zero, q is 1 and p is 2, or wherein p, q and n are each zero, r is 1 and m is 2 or 3.

In a preferred embodiment the compound of Formula (VIII) (ezetimibe): 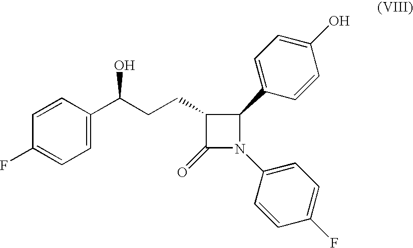
is used for the treatment of sitosterolemia.

In another embodiment, one or more sterol absorption inhibitors useful in the methods, compositions or combinations of this invention are represented by Formula (IX): 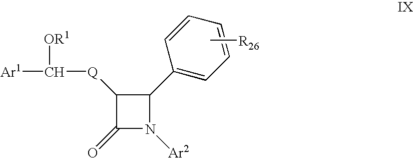
or isomers thereof, or pharmaceutically acceptable salts or solvates of the compounds of Formula (IX) or of the isomers thereof, or prodrugs of the compounds of Formula (IX) or of the isomers, salts or solvates thereof, wherein:

R²⁶ is selected from the group consisting of:
a) OH;
b) OCH₃;
c) fluorine and
d) chlorine.

R¹ is selected from the group consisting of
H, 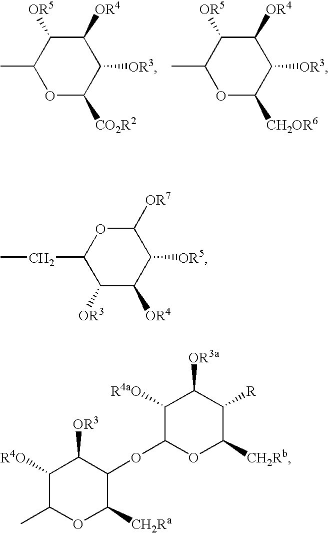
—SO₃H; natural and unnatural amino acids.

R, R^{a} and R^{b} are independently selected from the group consisting of H, —OH, halogeno, —NH₂, azido, (C₁-C₆)alkoxy(C₁-C₆)-alkoxy and —W—R³⁰;

W is independently selected from the group consisting of —NH—C(O)—, —O—C(O)—, —O—C(O)—N(R³¹)—, —NH—C(O)—N(R³¹)— and —O—C(S)—N(R³¹)—;

R² and R⁶ are independently selected from the group consisting of H, (C₁-C₆)alkyl, aryl and aryl(C₁-C₆)alkyl;

R³, R⁴, R⁵, R⁷, R₃ₐ and R^{4a} are independently selected from the group consisting of H, (C₁-C₆)alkyl, aryl(C₁-C₆)alkyl, —C(O)(C₁-C₆)alkyl and —C(O)aryl;

R³⁰ is independently selected form the group consisting of R³²-substituted T, R³²-substituted-T-(C₁-C₆)alkyl, R³²-substituted-(C₂-C₄)alkenyl, R³²-substituted-(C₁-C₆)alkyl, R³²-substituted-(C₃-C₇)cycloalkyl and R³²-substituted-(C₃-C₇)cycloalkyl(C₁-C₆)alkyl;

R³¹ is independently selected from the group consisting of H and (C₁-C₄)alkyl;

T is independently selected from the group consisting of phenyl, furyl, thienyl, pyrrolyl, oxazolyl, isoxazolyl, thiazolyl, iosthiazolyl, benzothiazolyl, thiadiazolyl, pyrazolyl, imidazolyl and pyridyl;

R³² is independently selected from 1-3 substituents independently selected from the group consisting of H, halogeno, (C₁-C₄)alkyl, —OH, phenoxy, —CF₃, —NO₂, (C₁-C₄)alkoxy, methylenedioxy, oxo, (C₁-C₄)alkylsulfanyl, (C₁-C₄)alkylsulfinyl, (C₁-C₄)alkylsulfonyl, —N(CH₃)₂, —C(O)—NH(C₁-C₄)alkyl, —C(O)—N((C₁-C₄)alkyl)₂, —C(O)—(C₁-C₄)alkyl, —C(O)—(C₁-C₄)alkoxy and pyrrolidinylcarbonyl; or R³² is a covalent bond and R³¹, the nitrogen to which it is attached and R³² form a pyrrolidinyl, piperidinyl, N-methyl-piperazinyl, indolinyl or morpholinyl group, or a (C₁-C₄)alkoxycarbonyl-substituted pyrrolidinyl, piperidinyl, N-methylpiperazinyl, indolinyl or morpholinyl group;

Ar¹ is aryl or R¹⁰-substituted aryl;

Ar² is aryl or R¹¹-substituted aryl;

Q is —(CH₂)_{q}—, wherein q is 2-6, or, with the 3-position ring carbon of the azetidinone,

forms the spiro group

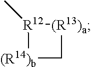

R¹² is

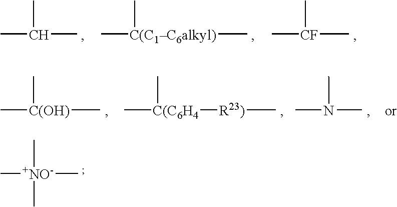

R¹³ and R¹⁴ are independently selected from the group consisting of —CH₂—, —CH(C₁-C₆ alkyl)-, —C(di-(C₁-C₆) alkyl), —CH═CH— and —C(C₁-C₆ alkyl)=CH—; or R¹² together with an adjacent R¹³, or R¹² together with an adjacent R¹⁴, form a —CH═CH— or a —CH═C(C₁-C₆ alkyl)- group;

a and b are independently 0, 1, 2 or 3, provided both are not zero; provided that when R¹³ is —CH═CH— or —C(C₁-C₆ alkyl)=CH—, a is 1; provided that when R¹⁴ is —CH═CH— or —C(C₁-C₆ alkyl)=CH—, b is 1; provided that when a is 2 or 3, the R¹³′s can be the same or different; and provided that when b is 2 or 3, the R¹⁴'s can be the same or different;

R¹⁰ and R¹¹ are independently selected from the group consisting of 1-3 substituents independently selected from the group consisting of (C₁-C₆)alkyl, —OR¹⁹, —O(CO)R¹⁹, —O(CO)OR²¹, —O(CH₂)₁₋₅OR¹⁹, —O(CO)NR¹⁹R²⁰, —NR¹⁹R²⁰, —NR¹⁹(CO)R²⁰, —NR¹⁹(CO)OR²¹, —NR¹⁹(CO)NR²OR²⁵, —NR¹⁹SO₂R²¹, —COOR¹⁹, —CONR¹⁹R²⁰, —COR¹⁹, —SO₂NR¹⁹R²⁰, S(O)₀₋₂R²¹, —O(CH₂)₁₋₁₀—COOR¹⁹, —O(CH₂)₁₋₁₀CONR¹⁹R²⁰, —(C₁-C₆ alkylene)-COOR¹⁹, —CH═CH—COOR¹⁹, —CF₃, —CN, —NO₂ and halogen;

Ar¹ can also be pyridyl, isoxazolyl, furanyl, pyrrolyl, thienyl, imidazolyl, pyrazolyl, thiazolyl, pyrazinyl, pyrimidinyl or pyridazinyl;

R¹⁹ and R²⁰ are independently selected from the group consisting of H, (C₁-C₆)alkyl, aryl and aryl-substituted (C₁-C₆)alkyl;

R²¹ is (C₁-C₆)alkyl, aryl or R²⁴-substituted aryl;

R²² is H, (C₁-C₆)alkyl, aryl (C₁-C₆)alkyl, —C(O)R¹⁹ or —COOR¹⁹;

R²³ and R²⁴ are independently ₁₋₃ groups independently selected from the group consisting of H, (C₁-C₆)alkyl, (C₁-C₆)alkoxy, —COOH, NO₂, —NR¹⁹R²⁰, —OH and halogeno; and

R²⁵ is H, —OH or (C₁-C₆)alkoxy.

Ar² is preferably phenyl or R¹¹-phenyl, especially (4-R¹¹)-substituted phenyl. Preferred definitions of R¹¹ are lower alkoxy, especially methoxy, and halogeno, especially fluoro.

Ar¹ is preferably phenyl or R¹⁰-substituted phenyl, especially (4-R¹⁰)— substituted phenyl. A preferred definition of R¹⁰ is halogeno, especially fluoro.

Preferably Q is a lower alkyl or a spiro group as defined above, wherein preferably R¹³ and R¹⁴ are each ethylene and R¹² is

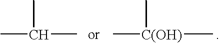

A preferred compound of formula IX, therefore, is one wherein R¹ is as defined above and in which the remaining variables have the following definitions:

Ar¹ is phenyl or R¹⁰-substituted phenyl, wherein R¹⁰ is halogeno;

Ar² is phenyl or R¹¹-phenyl, wherein R¹¹ is 1 to 3 substituents independently selected from the group consisting of C₁-C₆ alkoxy and halogeno;

Q is a lower alkyl (i.e C-1 to C-2) with Q=C-2 being preferred, or Q, with the 3-position ring carbon of the azetidinone, forms the group 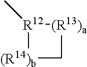
wherein preferably R¹³ and R¹⁴ are each ethylene and a and b are each 1, and wherein R¹² is

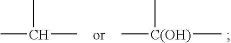

Preferred variables for R¹ groups of the formula 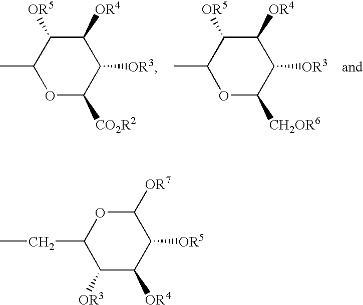
are as follows:

R², R³, R⁴, R⁵, R⁶ and R⁷ are independently selected from the group consisting of H, (C₁-C₆)alkyl, benzyl and acetyl;

Preferred variables for group R¹ of the formula 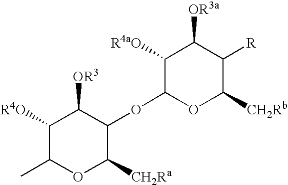
are as follows:

R³, R₃ₐ, R⁴ and R₄ₐ are selected from the group consisting of H, (C₁-C₆)alkyl, benzyl and acetyl;

R, R^{a} and R^{b} are independently selected from the group consisting of H, —OH, halogeno, —NH₂, azido, (C₁-C₆)alkoxy(C₁-C₆)alkoxy and —W—R³⁰, wherein W is —O—C(O)— or —O—C(O)—NR³¹—, R³¹ is H and R³⁰ is (C₁-C₆)alkyl, —C(O)—(C₁-C₄)alkoxy-(C₁-C₆)alkyl, T, T-(C₁-C₆)alkyl, or T or T-(C₁-C₆)alkyl wherein T is substituted by one or two halogeno or (C₁-C₆)alkyl groups.

Preferred R³⁰ substituents are 2-fluorophenyl, 2,4-difluoro-phenyl, 2,6-dichlorophenyl, 2-methylphenyl, 2-thienylmethyl, 2-methoxy-carbonylethyl, thiazol-2-yl-methyl, 2-furyl, 2-methoxycarbonylbutyl and phenyl. Preferred combinations of R, R^{a} and R^{b} are as follows: 1) R, R^{a} and R^{b} are independently —OH or —O—C(O)—NH—R³⁰, especially wherein R^{a} is —OH and R and R^{b} are —O—C(O)—NH—R³⁰ and R³⁰ is selected from the preferred substituents identified above, or wherein R and R^{a} are —OH and R^{b} is-O—C(O)—NH—R³⁰ wherein R³⁰ is 2-fluorophenyl, 2,4-difluoro-phenyl, 2,6-dichlorophenyl; 2) R^{a} is —OH, halogeno, azido or (C₁-C₆)-alkoxy(C₁-C₆)alkoxy, R^{b} is H, halogeno, azido or (C₁-C₆)alkoxy(C₁-C₆)-alkoxy, and R is —O—C(O)—NH—R³⁰, especially compounds wherein R^{a} is —OH, R^{b} is H and R³⁰ is 2-fluorophenyl; 3) R, R^{a} and R^{b} are independently —OH or —O—C(O)—R³⁰ and R³⁰ is (C₁-C₆)alkyl, T, or T substituted by one or two halogeno or (C₁-C₆)alkyl groups, especially compounds wherein R is —OH and R^{a} and R^{b} are —O—C(O)—R³⁰ wherein R³⁰ is 2-furyl; and 4) R, R^{a} and R^{b} are independently —OH or halogeno. Three additional classes of preferred are compounds are those wherein the C^{1′} anomeric oxy is beta, wherein the C^{2′} anomeric oxy is beta, and wherein the R group is alpha.

R¹ is preferably selected from: 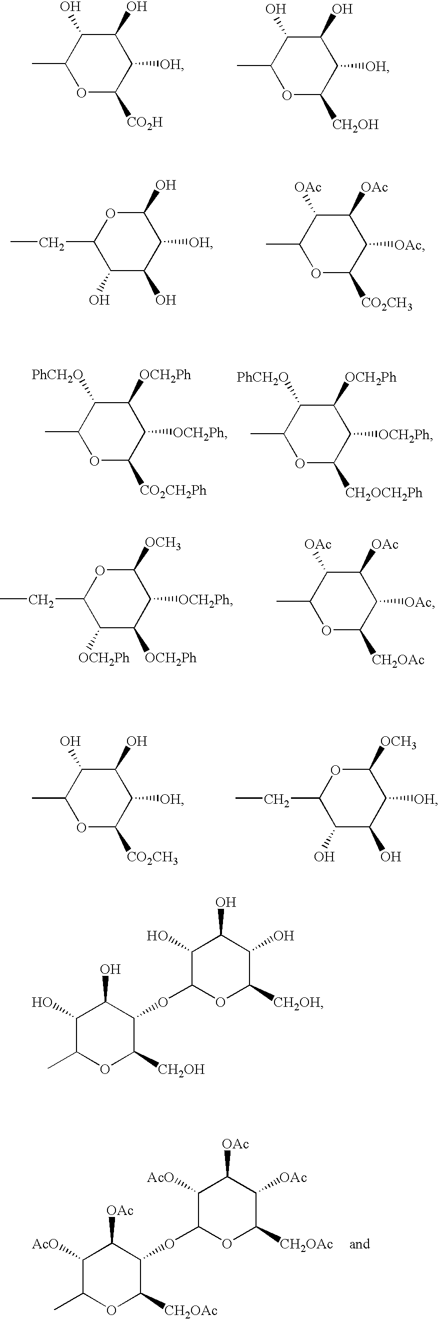 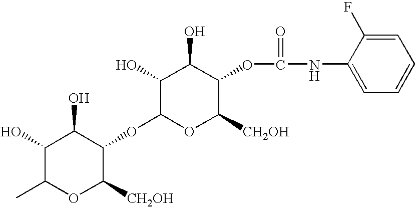
wherein Ac is acetyl and Ph is phenyl.

Thus a preferred compound of this invention is one represented by the formula X: 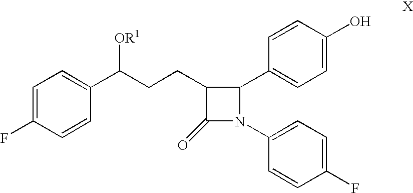
wherein R¹ is defined as above.

A more preferred compound is one represented by formula XI:

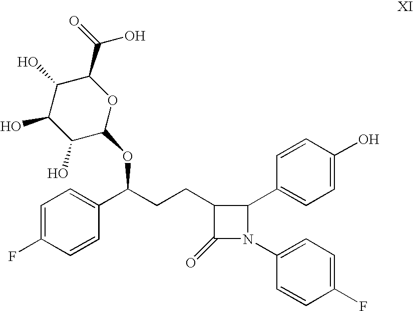

Methods for making the compounds described above and other non-limiting examples of suitable compounds useful in the treatment of sitosterolemia, are well known to those skilled in the art. Their general preparation and specific examples are disclosed in U.S. Pat. Nos. 5,767,115; 5,846,966; 5,756,470, 5,698,548; 5,624,920; 5,656,624; 5,688,787; 5,688,990, 5,631,365, 6,207,822 and U.S. Provisional Patent Application 60/279,288 filed Mar. 28, 2001, each of which is incorporated herein by reference.

Generally, compounds of formulas I-XI can be prepared by known methods, for example WO 93/02048 describes the preparation of compounds wherein —R¹-Q- is alkylene, alkenylene or alkylene interrupted by a hetero atom, phenylene or cycloalkylene; WO 94/17038 describes the preparation of compounds wherein Q is a spirocyclic group; WO 95/08532 describes the preparation of compounds wherein —R¹-Q- is a hydroxy-substituted alkylene group; PCT/US95/03196 describes compounds wherein —R¹-Q- is a hydroxy-substituted alkylene attached to the Ar¹ moiety through an —O— or S(O)₀₋₂— group; and U.S. Ser. No. 08/463,619, filed Jun. 5, 1995, describes the preparation of compounds wherein —R¹-Q- is a hydroxy-substituted alkylene group attached the the azetidinone ring by a —S(O)₀₋₂- group, each of which is incorporated herein by reference.

As used herein, the term “alkyl” or “lower alkyl” means straight or branched alkyl chains of 1 to 6 carbon atoms and “alkoxy” similarly refers to alkoxy groups having 1 to 6 carbon atoms.

“Alkenyl” means straight or branched carbon chains having one or more double bonds in the chain, conjugated or unconjugated. Similarly, “alkynyl” means straight or branched carbon chains having one or more triple bonds in the chain. Where an alkyl, alkenyl or alkynyl chain joins two other variables and is therefore bivalent, the terms alkylene, alkenylene and alkynylene are used.

“Cycloalkyl” means a saturated carbon ring of 3 to 6 carbon atoms, while “cycloalkylene” refers to a corresponding bivalent ring, wherein the points of attachment to other groups include all positional isomers.

“Halogeno” refers to fluorine, chlorine, bromine or iodine radicals.

“Aryl” means phenyl, naphthyl, indenyl, tetrahydronaphthyl or indanyl.

“Phenylene” means a bivalent phenyl group, including ortho, meta and para-substitution.

As used herein, “prodrug” means compounds that are drug precursors which, following administration to a patient, release the drug in vivo via some chemical or physiological process (e.g., a prodrug on being brought to the physiological pH or through enzyme action is converted to the desired drug form).

As used herein, “solvate” means a molecular or ionic complex of molecules or ions of solvent with those of solute (for example, one or more compounds of Formula I-XI, isomers of the compounds of Formula I-XI, and prodrugs of the compounds of Formula I-XI). Non-limiting examples of useful solvents include polar, protic solvents such as water and alcohols (for example methanol).

The statements, wherein, for example, R¹⁹, R²⁰ and R²⁵ are said to be independently selected from a group of substituents, means that R¹⁹, R²⁰ and R²⁵ are independently selected, but also that where an R¹⁹, R²⁰ or R²⁵ variable occurs more than once in a molecule, those occurrences are independently selected (e.g., if R¹⁰ is —OR¹⁹ wherein R¹⁹ is hydrogen, R¹¹ can be —OR¹⁹ wherein R¹⁹ is lower alkyl). Those skilled in the art will recognize that the size and nature of the substituent(s) will affect the number of substituents which can be present.

The term “therapeutically effective amount” means that amount of a therapeutic agent of the composition, such as the bile acid sequestrant(s), sterol absorption inhibitor(s) and other pharmacological or therapeutic agents described below, that will elicit a biological or medical response of a tissue, system, animal or mammal that is being sought by the administrator (such as a researcher, doctor or veterinarian) which includes alleviation of the symptoms of the sitosterolemic condition or disease being treated and the prevention, slowing or halting of progression of the sitosterolemic condition.

As used herein, “combination therapy” or “therapeutic combination” means the administration of two or more therapeutic agents, such as sterol absorption inhibitor(s) and bile acid sequestrant(s) or other therapeutic vascular agents, to prevent or treat sitosterolemia and/or reduce the level of sterol(s) in the plasma and tissues. As used herein, “vascular” comprises cardiovascular, cerebrovascular and combinations thereof. Such administration includes coadministration of these therapeutic agents in a substantially simultaneous manner, such as in a single tablet or capsule having a fixed ratio of active ingredients or in multiple, separate capsules for each therapeutic agent. Also, such administration includes use of each type of therapeutic agent in a sequential manner. In either case, the treatment using the combination therapy will provide beneficial effects in treating the sitosterolemic condition. A potential advantage of the combination therapy disclosed herein may be a reduction in the required amount of an individual therapeutic compound or the overall total amount of therapeutic compounds that are effective in treating the sitosterolemic condition. Therapeutic agents can be selected to provide a broader range of complementary effects or complimentary modes of action.

Compounds of the invention have at least one asymmetrical carbon atom and therefore all isomers, including enantiomers, stereoisomers, rotamers, tautomers, racemates of the compounds of Formula (I-XI) (where they exist) are contemplated as being part of this invention. The invention includes d and I isomers in both pure form and in admixture, including racemic mixtures. Isomers can be prepared using conventional techniques, either by reacting optically pure or optically enriched starting materials or by separating isomers of a compound of the Formulae I-XI. Isomers may also include geometric isomers, e.g., when a double bond is present.

Those skilled in the art will appreciate that for some of the compounds of the Formulas I-XI, one isomer will show greater pharmacological activity than other isomers.

Compounds of the invention with an amino group can form pharmaceutically acceptable salts with organic and inorganic acids. Examples of suitable acids for salt formation are hydrochloric, sulfuric, phosphoric, acetic, citric, oxalic, malonic, salicylic, malic, fumaric, succinic, ascorbic, maleic, methanesulfonic and other mineral and carboxylic acids well known to those in the art. The salt is prepared by contacting the free base form with a sufficient amount of the desired acid to produce a salt. The free base form may be regenerated by treating the salt with a suitable dilute aqueous base solution such as dilute aqueous sodium bicarbonate. The free base form differs from its respective salt form somewhat in certain physical properties, such as solubility in polar solvents, but the salt is otherwise equivalent to its respective free base forms for purposes of the invention.

Certain compounds of the invention are acidic (e.g., those compounds which possess a carboxyl group). These compounds form pharmaceutically acceptable salts with inorganic and organic bases. Examples of such salts are the sodium, potassium, calcium, aluminum, gold and silver salts. Also included are salts formed with pharmaceutically acceptable amines such as ammonia, alkyl amines, hydroxyalkylamines, N-methylglucamine and the like.

In an alternative embodiment, the treatment composition can further comprise one or more bile acid sequestrant(s) in coadministration with or in combination with one or more sterol absorption inhibitors.

Non-limiting examples of suitable bile acid sequestrants include cholestyramine (a styrene-divinylbenzene copolymer containing quaternary ammonium cationic groups capable of binding bile acids, such as QUESTRAN® or QUESTRAN LIGHT® which are available from Bristol-Myers Squibb), colestipol (a copolymer of diethylenetriamine and 1-chloro-2,3-epoxypropane, such as COLESTID® tablets which are available from Pharmacia), colesevelam hydrochloride (such as WelChol® Tablets (poly(allylamine hydrochloride) cross-linked with epichlorohydrin and alkylated with 1-bromodecane and (6-bromohexyl)-trimethylammonium bromide) which are available from Sankyo), water soluble derivatives such as 3,3-ioene, N-(cycloalkyl) alkylamines and poliglusam, insoluble quaternized polystyrenes, saponins and mixtures thereof. Other useful bile acid sequestrants are disclosed in PCT Patent Applications Nos. WO 97/11345 and WO 98/57652, and U.S. Pat. Nos. 3,692,895 and 5,703,188 which are incorporated herein by reference. Suitable inorganic cholesterol sequestrants include bismuth salicylate plus montmorillonite clay, aluminum hydroxide and calcium carbonate antacids.

The bile acid sequestrant(s) are administered in a therapeutically effective amount to treat the specified condition, for example in a daily dose preferably ranging from about 1 to about 50 grams per day, and more preferably about 2 to about 16 grams per day, given in a single dose or 2-4 divided doses. The exact dose, however, is determined by the attending clinician and is dependent on such factors as the potency of the compound administered, the age, weight, condition and response of the patient.

In yet another alternative embodiment, the treatment composition can further comprise one or more lipid lowering agents such as, for example, sterol biosynthesis inhibitors, in coadministration with or in combination with one or more sterol absorption inhibitors.

Non-limiting lipid lowering agents for use in the treatment compositions of the present invention include HMG CoA reductase inhibitors such as lovastatin, pravastatin, fluvastatin, simvastatin, atorvastatin, rosuvastatin and itavastatin. Preferred HMG CoA reductase inhibitors include lovastatin, atorvastatin and simvastatin. The most preferred HMG CoA reductase inhibitors are atorvastatin and simvastatin.

In another preferred embodiment, the treatment composition comprises the compound of Formula (VIII) in combination with a bile acid sequestrant. In this embodiment, preferably the bile acid sequestrant is selected from cholestyramine, colesevelam hydrochloride and colestipol. Preferably, the treatment composition comprises one or more bile acid sequestrants such as, for example, cholestyramine, colesevelam hydrochloride and colestipol in combination with a compound of Formula (VIII)

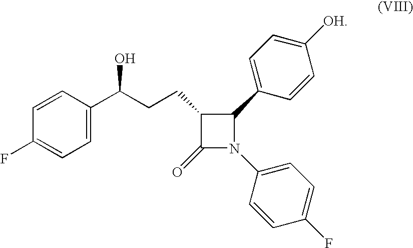

In another preferred embodiment, the treatment composition comprises the compound of Formula (VIII) in combination with another lipid lowering agent. In this embodiment, preferably the lipid lowering agent comprises one or more HMG CoA reductase inhibitors. Preferably, the treatment composition comprises one or more HMG CoA reductase inhibitors such as, for example, lovastatin, atorvastatin and simvastatin in combination with a compound of Formula (VIII)

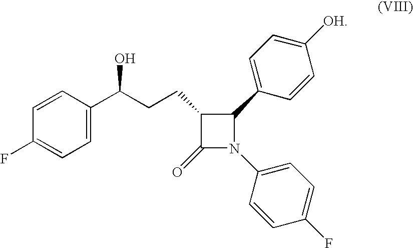

Still even more preferred, the treatment composition comprises compound of formula VIII in combination with atorvastatin and/or simvastatin.

In one embodiment of the invention, the compositions or therapeutic combinations can further comprise one or more pharmacological or therapeutic agents or drugs such as cholesterol biosynthesis inhibitors and/or lipid-lowering agents discussed below.

Also useful with the invention are compositions or therapeutic combinations that can further comprise at least one (one or more) activators for peroxisome proliferator-activated receptors (PPAR). The activators act as agonists for the peroxisome proliferator-activated receptors. Three subtypes of PPAR have been identified, and these are designated as peroxisome proliferator-activated receptor alpha (PPARα), peroxisome proliferator-activated receptor gamma (PPARγ) and peroxisome proliferator-activated receptor delta (PPARδ). It should be noted that PPARδ is also referred to in the literature as PPARδ and as NUC1, and each of these names refers to the same receptor.

PPARα regulates the metabolism of lipids. PPARα is activated by fibrates and a number of medium and long-chain fatty acids, and it is involved in stimulating β-oxidation of fatty acids. The PPARγ receptor subtypes are involved in activating the program of adipocyte differentiation and are not involved in stimulating peroxisome proliferation in the liver. PPARδ has been identified as being useful in increasing high density lipoprotein (HDL) levels in humans. See, e.g., WO 97/28149.

PPARα activator compounds are useful for, among other things, lowering triglycerides, moderately lowering LDL levels and increasing HDL levels. Useful examples of PPARα activators include fibric acid derivatives or fibrates.

Non-limiting examples of suitable fibric acid derivatives (“fibrates”) include clofibrate (such as ethyl 2-(p-chlorophenoxy)-2-methyl-propionate, for example ATROMID-S® Capsules which are commercially available from Wyeth-Ayerst); gemfibrozil (such as 5-(2,5-dimethylphenoxy)-2,2-dimethylpentanoic acid, for example LOPID® tablets which are commercially available from Parke Davis); ciprofibrate (C.A.S. Registry No. 52214-84-3, see U.S. Pat. No. 3,948,973 which is incorporated herein by reference); bezafibrate (C.A.S. Registry No. 41859-67-0, see U.S. Pat. No. 3,781,328 which is incorporated herein by reference); clinofibrate (C.A.S. Registry No. 30299-08-2, see U.S. Pat. No. 3,716,583 which is incorporated herein by reference); binifibrate (C.A.S. Registry No. 69047-39-8, see BE 884722 which is incorporated herein by reference); lifibrol (C.A.S. Registry No. 96609-16-4); fenofibrate (such as TRICOR® micronized fenofibrate (2-[4-(4-chlorobenzoyl) phenoxy]-2-methyl-propanoic acid, 1-methylethyl ester) which is commercially available from Abbott Laboratories or LIPANTHYL® micronized fenofibrate which is commercially available from Labortoire Founier, France) and mixtures thereof. These compounds can be used in a variety of forms, including but not limited to acid form, salt form, racemates, enantiomers, zwitterions and tautomers.

Other examples of PPARα activators useful with the practice of the present invention include suitable fluorophenyl compounds as disclosed in U.S. Pat. No. 6,028,109 which is incorporated herein by reference; certain substituted phenylpropionic compounds as disclosed in WO 00/75103 which is incorporated herein by reference; and PPARα activator compounds as disclosed in WO 98/43081 which is incorporated herein by reference.

Non-limiting examples of suitable PPARγ activators include derivatives of glitazones or thiazolidinediones, such as, troglitazone (such as REZULIN® troglitazone (-5-[[4-[3,4-dihydro-6-hydroxy-2,5,7,8-tetramethyl-2H-1-benzopyran-2-yl)methoxy]phenyl]methyl]-2,4-thiazolidinedione) commercially available from Parke-Davis); rosiglitazone (such as AVANDIA® rosiglitazone maleate (-5-[[4-[2-(methyl-2-pyridinylamino)ethoxy]phenyl]methyl]-2,4-thiazolidinedione, (Z)-2-butenedioate) commercially available from SmithKline Beecham) and pioglitazone (such as ACTOS™ pioglitazone hydrochloride (5-[[4-[2-(5-ethyl-2-pyridinyl)ethoxy]phenyl]methyl]-2,4-thiazolidinedione monohydrochloride) commercially available from Takeda Pharmaceuticals). Other useful thiazolidinediones include ciglitazone, englitazone, darglitazone and BRL 49653 as disclosed in WO 98/05331 which is incorporated herein by reference; PPARγ activator compounds disclosed in WO 00/76488 which is incorporated herein by reference; and PPARγ activator compounds disclosed in U.S. Pat. No. 5,994,554 which is incorporated herein by reference.

Other useful PPARγ activator compounds include certain acetylphenols as disclosed in U.S. Pat. No. 5,859,051 which is incorporated herein by reference; certain quinoline phenyl compounds as disclosed in WO 99/20275 which is incorporated herein by reference; aryl compounds as disclosed by WO 99/38845 which is incorporated herein by reference; certain 1,4-disubstituted phenyl compounds as disclosed in WO 00/63161; certain aryl compounds as disclosed in WO 01/00579 which is incorporated herein by reference; benzoic acid compounds as disclosed in WO 01/12612 and WO 01/12187 which are incorporated herein by reference; and substituted 4-hydroxy-phenylalconic acid compounds as disclosed in WO 97/31907 which is incorporated herein by reference.

PPARδ compounds are useful for, among other things, lowering triglyceride levels or raising HDL levels. Non-limiting examples of PPARδ activators include suitable thiazole and oxazole derivates, such as C.A.S. Registry No. 317318-32-4, as disclosed in WO 01/00603 which is incorporated herein by reference); certain fluoro, chloro or thio phenoxy phenylacetic acids as disclosed in WO 97/28149 which is incorporated herein by reference; suitable non-β-oxidizable fatty acid analogues as disclosed in U.S. Pat. No. 5,093,365 which is incorporated herein by reference; and PPARδ compounds as disclosed in WO 99/04815 which is incorporated herein by reference.

Moreover, compounds that have multiple functionality for activating various combinations of PPARα, PPARγ and PPARδ are also useful with the practice of the invention. Non-limiting examples include certain substituted aryl compounds as disclosed in U.S. Pat. No. 6,248,781; WO 00/23416; WO 00/23415; WO 00/23425; WO 00/23445; WO 00/23451; and WO 00/63153, all of which are incorporated herein by reference, are described as being useful PPARα and/or PPARγ activator compounds. Other non-limiting examples of useful PPARα and/or PPARγ activator compounds include activator compounds as disclosed in WO 97/25042 which is incorporated herein by reference; activator compounds as disclosed in WO 00/63190 which is incorporated herein by reference; activator compounds as disclosed in WO 01/21181 which is incorporated herein by reference; biaryl-oxa(thia)zole compounds as disclosed in WO 01/16120 which is incorporated herein by reference; compounds as disclosed in WO 00/63196 and WO 00/63209 which are incorporated herein by reference; substituted 5-aryl-2,4-thiazolidinediones compounds as disclosed in U.S. Pat. No. 6,008,237 which is incorporated herein by reference; arylthiazolidinedione and aryloxazolidinedione compounds as disclosed in WO 00/78312 and WO 00/78313G which are incorporated herein by reference; GW2331 or (2-(4-[difluorophenyl]-1 heptylureido)ethyl]phenoxy)-2-methylbutyric compounds as disclosed in WO 98/05331 which is incorporated herein by reference; aryl compounds as disclosed in U.S. Pat. No. 6,166,049 which is incorporated herein by reference; oxazole compounds as disclosed in WO 01/17994 which is incorporated herein by reference; and dithiolane compounds as disclosed in WO 01/25225 and WO 01/25226 which are incorporated herein by reference.

Other useful PPAR activator compounds include substituted benzylthiazolidine-2,4-dione compounds as disclosed in WO 01/14349, WO 01/14350 and WO/01/04351 which are incorporated herein by reference; mercaptocarboxylic compounds as disclosed in WO 00/50392 which is incorporated herein by reference; ascofuranone compounds as disclosed in WO 00/53563 which is incorporated herein by reference; carboxylic compounds as disclosed in WO 99/46232 which is incorporated herein by reference; compounds as disclosed in WO 99/12534 which is incorporated herein by reference; benzene compounds as disclosed in WO 99/15520 which is incorporated herein by reference; o-anisamide compounds as disclosed in WO 01/21578 which is incorporated herein by reference; and PPAR activator compounds as disclosed in WO 01/40192 which is incorporated herein by reference.

The peroxisome proliferator-activated receptor(s) activator(s) are administered in a therapeutically effective amount to treat the specified condition, for example in a daily dose preferably ranging from about 50 to about 3000 mg per day, and more preferably about 50 to about 2000 mg per day, given in a single dose or 24 divided doses. The exact dose, however, is determined by the attending clinician and is dependent on such factors as the potency of the compound administered, the age, weight, condition and response of the patient.

In an alternative embodiment, the compositions or therapeutic combinations of the invention can further comprise one or more ileal bile acid transport (“IBAT”) inhibitors (or apical sodium co-dependent bile acid transport (“ASBT”) inhibitors) coadministered with or in combination with the sterol absorption inhibitor(s) discussed above. The IBAT inhibitors can inhibit bile acid transport to reduce LDL cholesterol levels. Non-limiting examples of suitable IBAT inhibitors include benzothiepines such as therapeutic compounds comprising a 2,3,4,5-tetrahydro-1-benzothiepine 1,1-dioxide structure such as are disclosed in PCT Patent Application WO 00/38727 which is incorporated herein by reference.

Generally, a total daily dosage of IBAT inhibitor(s) can range from about 0.01 to about 1000 mg/day, and preferably about 0.1 to about 50 mg/day in single or 24 divided doses.

In another alternative embodiment, the compositions or therapeutic combinations of the invention can further comprise nicotinic acid (niacin) and/or derivatives thereof coadministered with or in combination with the sterol absorption inhibitor(s) discussed above.

As used herein, “nicotinic acid derivative” means a compound comprising a pyridine-3-carboxylate structure or a pyrazine-2-carboxylate structure, including acid forms, salts, esters, zwitterions and tautomers, where available. Examples of nicotinic acid derivatives include niceritrol, nicofuranose and acipimox (5-methylpyrazine-2-carboxylic acid 4-oxide). Nicotinic acid and its derivatives inhibit hepatic production of VLDL and its metabolite LDL and increases HDL and apo A-1 levels. An example of a suitable nicotinic acid product is NIASPAN® (niacin extended-release tablets) which are available from Kos.

Generally, a total daily dosage of nicotinic acid or a derivative thereof can range from about 500 to about 10,000 mg/day, preferably about 1000 to about 8000 mg/day, and more preferably about 3000 to about 6000 mg/day in single or divided doses.

In another alternative embodiment, the compositions or therapeutic combinations of the invention can further comprise one or more AcylCoA:Cholesterol O-acyltransferase (“ACAT”) Inhibitors, which can reduce LDL and VLDL levels, coadministered with or in combination with the sterol absorption inhibitor(s) discussed above. ACAT is an enzyme responsible for esterifying excess intracellular cholesterol and may reduce the synthesis of VLDL, which is a product of cholesterol esterification, and overproduction of apo B-100-containing lipoproteins.

Non-limiting examples of useful ACAT inhibitors include avasimibe ([[2,4,6-tris(1-methylethyl)phenyl]acetyl]sulfamic acid, 2,6-bis(1-methylethyl)phenyl ester, formerly known as CI-1011), HL-004, lecimibide (DuP-128) and CL-277082 (N-(2,4-difluorophenyl)-N-[[4-(2,2-dimethylpropyl)phenyl]methyl]-N-heptylurea). See P. Chang et al., “Current, New and Future Treatments in Dyslipidaemia and Atherosclerosis”, Drugs 2000 July; 60(1); 55-93, which is incorporated by reference herein.

Generally, a total daily dosage of ACAT inhibitor(s) can range from about 0.1 to about 1000 mg/day in single or 2-4 divided doses.

In another alternative embodiment, the compositions or therapeutic combinations of the invention can further comprise one or more Cholesteryl Ester Transfer Protein (“CETP”) Inhibitors coadministered with or in combination with the sterol absorption inhibitor(s) discussed above. CETP is responsible for the exchange or transfer of cholesteryl ester carrying HDL and triglycerides in VLDL.

Non-limiting examples of suitable CETP inhibitors are disclosed in PCT Patent Application No. WO 00/38721, WO2005/095409, WO2000/176164, WO2000/17165, WO2000/17166 and U.S. Pat. No. 6,147,090, which are incorporated herein by reference as well as the documents cited in these publications are also incorporated by reference. Pancreatic cholesteryl ester hydrolase (pCEH) inhibitors such as WAY-121898 also can be coadministered with or in combination with the peroxisome proliferator-activated receptor(s) activator and sterol absorption inhibitor(s) discussed above.

Especially preferred CEPT inhibitors which may be used in pharmaceutical combinations with the sterol absorption inhibitor(s) discussed above are tetrahydroquinoline derivatives of the formula (I′): 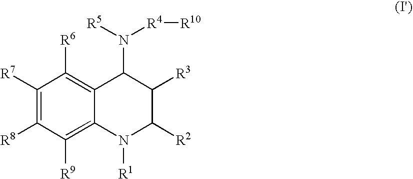
wherein R¹ is a hydrogen atom, an optionally substituted alkoxycarbonyl group, an optionally substituted carbamoyl group, an optionally substituted alkyl group, an optionally substituted alkanoyl group, a saturated or unsaturated monocyclic or bicyclic heterocyclic group containing 1 to 4 heteroatoms selected independently from oxygen, sulfur and nitrogen atoms (the heterocyclic group is optionally substituted), or a saturated or unsaturated monocyclic or bicylic heterocyclic carbonyl group containing 1 to 4 heteroatoms selected independently from oxygen, sulfur and nitrogen atoms (the heterocyclic group is optionally substituted);
R² is a hydrogen atom or an optionally substituted alkyl group;
R³ is a hydrogen atom or an optionally substituted alkyl group;
R⁴ is an optionally substituted alkylene group;
R⁵ is a saturated or unsaturated monocyclic or bicyclic heterocyclic group containing 1 to 4 heteroatoms selected independently from oxygen, sulfur and nitrogen atoms, wherein the heterocyclic group is substituted by 1 to 5 substituents selected from the following groups, or said heterocyclic group is substituted by 1 to 5 substituents selected from the following groups and further by a halogen atom, an oxo and/or hydroxyl group;
   cyano group, nitro group, carboxyl group, sulfo group, C₃₋₁₀ alkyl group, substituted alkyl group, optionally substituted cycloalkyl group, optionally substituted alkenyl group, C₃₋₁₀ alkoxy group, substituted alkoxy group, optionally substituted cycloalkoxy group, optionally substituted alkoxycarbonyl group, optionally substituted carbamoyl group, optionally substituted carbamimidoyl group, optionally substituted alkylthio group, optionally substituted alkylsulfinyl group, optionally substituted alkylsulfonyl group, optionally substituted allkylsuflonyl group, optionally substituted amino group, optionally substituted sulfamoyl group, optionally substituted alkanoyl group, a saturated or unsaturated monocyclic or bicyclic heterocyclic group containing 1 to 4 heteroatoms selected independently from oxygen, sulfur and nitrogen atoms (the heterocyclic group is optionally substituted), a saturated or unsaturated monocyclic or bicyclic heterocyclic oxy group containing 1 to 4 heteroatoms selected independently from oxygen, sulfur and nitrogen atoms (the heterocyclic oxy group is optionally substituted), and a saturated or unsaturated monocyclic or bicyclic heterocyclic carbonyl group containing 1 to 4 heterocyclic carbonyl group is optionally substituted);
R⁶ and R⁷, or R⁷ and R₈, or R⁸ and R⁹ may combine at the ends to form an alkylene group which alkylene group may contain 1 to 3 heteroatoms selected independently from nitrogen, sulfur and oxygen atoms, and may have a substituent(s); and
R₁₀ is an aromatic ring optionally containing 1 to 3 heteroatoms selected independently from oxygen, sulfur and nitrogen atoms (the aromatic ring is optionally substituted),
   or a pharmaceutically acceptable salt thereof. The definitions for compounds of formula (I′) are those described in WO 2005/095409. The compound (I′) of the present invention encompasses a mixture of stereoisomers, respective stereoisomers in a purified or substantially purified form.

Another embodiment of the present invention include compounds of the formula (I) wherein R⁵ is a group of the formula: 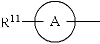
wherein Ring A is a saturated or unsaturated 5- to 8-membered heterocyclic group containing 1 to 4 heteroatoms selected independently from oxygen, sulfur and nitrogen atoms, and
R¹¹ is a group selected from the following groups:
cyano group;
nitro group;
carboxyl group;
sulfo group;
alkyl group substituted by a group selected from halogen atom, cyano group, hydroxyl group, carboxyl group, alkoxycarbonyl group, alkoxy group (said alkoxy group is optionally substituted by phenyl, hydroxyl or carboxyl group), alkanoyloxy group, alkylthio group, alkylsulfonyl group, alkylsulfinyl group, amino group, mono- or di-alkylamino group, mono- or di-alkylsulfamoylamino group, mono- or di-alkylureido group optionally substituted by morpholinyl group, oxiranyl group, dialkyldioxolanyl group, pyrrolidinyl grouop optionally substituted by alkoxycarbonyl or carboxyl group, piperidinyl group optionally substituted by alkoxycarbonyl or carboxyl group, piperazinyl group optionally substituted by alkyl group, hexahydroazepinyl grouop and morpholinyl group;
alkenyl group optionally substituted by carboxyl group, cyano group or benzyloxycarbonyl group;
alkoxy grouop substituted by a group selected from halogen atom, cyano group, hydroxyl group, carboxyl group, alkoxycarbonyl group, alkoxy group (said alkoxy group is optionally substituted by phenyl, hydroxyl or carboxyl group), alkanoyloxy group, alkylthio group, alkylsulfonyl group, mono- or di-alkylsulfamoylamino group, mono- or di-alkylureido group optionally substituted by morpholinyl group, oxiranyl group, di-alkyldioxolanyl group, pyrrolidinyl group optionally substituted by alkoxycarbonyl or carboxyl group, piperidinyl group optionally substituted by alkoxycarbonyl or carboxyl group, piperazinyl group optionally substituted by alkyl group, hexahydroazepinyl group and morpholinyl group;
alkoxycarbonyl group optionally substituted by phenyl group; mono- or di-alkylcarbamoyl group optionally substituted by carboxyl group;
hydroxycarbamimidoyl group;
alkylthio group;
alkylsulfinyl group;
alkylsulfonyl group optionally substituted by carboxyl group;
mono- or di-alkylamino group optionally substituted by hydroxyl group, carboxyl group, alkoxycarbonyl group, alkoxy group, mono- or di-alkylamino group or morpholinyl group;
mono- or di-alkylsulfamoyl group;
morpholinyl group;
optionally oxidized thiomorpholinyl group;
piperazinyl group optionally substituted by a group selected from alkyl group, alkanoyl group and hydroxyalkanoyl group;
pyrrolidinyl group optionally substituted by carboxyl group, carboxyalkyl group, alkyl group, alkoxycarbonyl group or alkoxycarbonylalkyl group; dioxolanyl group optionally substituted by alkyl group;
tetrazolyl group optionally substituted by alkyl group, hydroxyalkl group or morpholinylalkyl group;
dihydrooxadiazolyl group optionally substituted by oxo group;
pyrimidinyl group; or
tetrahydropyranyl group;

which compound is shown by the formula (I′-A):

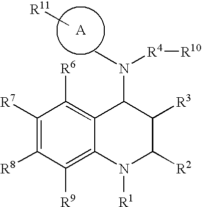

where each symbol has the same meaning as defined above.

More preferred embodiment includes compounds of the formula (I) where R¹ is a hydrogen atom; an alkoxycarbonyl group optionally substituted by 1 to 5 substituents selected independently from cyano group, hydroxyl group, carboxyl group, alkoxycarbonyl group, alkoxy group (said alkoxy group is optionally substituted by 1 to 3 substituents selected independently from hydroxy grouop, amino group, mono- or di-alkylamino group, halogen atom, carboxyl grop, alkoxycarbonyl group, carboxyalkoxy group and alkoxycarbonylalkoxy group), alkylthio group, alkylsulfonyl group, alkenyl group, amino group, mono- or di-alkylamino group, tetrazolyl group, carbamoyl group, mono- or di-alkylcarbamoyl group (said mono- or di-alkylcarbamoyl group is optionally substituted by 1 to 3 substituents selected independently from carboxyl group and alkoxycarbonyl group), alkanoylamino group (said alkanoylamino group is optionally substituted by 1 to 3 substituents selected independently from carboxyl group, alkoxycarbonyl group, hydroxyl group and halogen atom), halogen atom, cycloalkyl grop (said cycloalkyl group is optionally substituted by 1 to 3 substituents selected independently from hydroxyl group, amino group, mono- or di-alkylamino group, halogen atom, oxo group, carboxyl group, alkoxycarbonyl group, carboxyalkyl group, alkoxcarbonylalkyl group, carboxyalkoxy group and alkoxycarbonylalkoxy group), phenyl group (said phenyl group is optionally substituted by 1 to 3 substitutents selected independently from hydroxyl group, amino group, mono- or di-alkylamino group, halogen atom, carboxyl group, alkoxycarbonyl group, carboxyalkyl group, alkoxcarbonylalkyl group, carboxyalkoxy group and alkoxycarbonylalkoxy group), morpholinyl group optionally substituted by oxo group, piperidinyl group (said piperidinyl group is optionally substituted by 1 to 3 substituents selected indepdently from hydroxyl group, amino group, mono- or di-alkylamino group, halogen atom, oxo grouop, carboxyl group, alkoxycarbonyl group, carboxyalkyl group, alkoxycarbonylalkyl group and carboxyalkoxy group, alkoxycarbonylalkoxy group), pyrrolidinyl group (said pyrrolidinyl group is optionally substituted by 1 to 3 substitutents selected independently from hydroxy group, amino group, mono- or di-alkylamino group, halogen atom, oxo group, carboxyl group, alkoxycarbonyl group and alkoxycarbonylalkoxy group), and pyrimidinyl group (said pyrimidinyl group is optionally substituted by 1 to 3 substituents selected independently from carboxyalkyl group, alkoxycarbonylalkyl group and carboxyalkoxy group, alkoxycarbonylalkoxy group); dihydrooxazolyl group optionally substituted by 1 to 2 substituents selected independently from carboxyl group, alkoxycarbonyl group, alkyl group, carboxyalkyl group, alkoxycarbonylalkyl group and hydroxyalkyl group; or a mono- or di-alkylcarbamoyl group optionally substituted by 1 to 5 substituents selected independently from halogen atom, hydroxy group, carboxyl group, alkoxycarbonyl group, alkoxy group, amino group, mono- or di-alkylamino group, morpholinyl group, pyridyl group, cycloalkyl group (said cycloalkyl group is optionally substituted by 1 to 3 substituents selected independently from carboxyl group, alkoxycarbonyl group, halogen atom, amino group and hydroxyl group) and phenyl group (said phenyl group is optionally substituted by 1 to 3 substituents selected independently from carboxyl group, alkoxycarbonyl group, halogen atom, amino group and hydroxyl group);
R² is an alkyl group;
R³ is a hydrogen atom;
R⁴ is an alkylene group;
Ring A and R¹¹ are the same groups as defined above;
R⁶ and R⁹ each are a hydrogen atom;
R⁷ and R⁸ are independently a hydrogen atom, an alkyl group optionally substituted by halogen atom, alkoxy group, or mono- or di-alkylamino group; or combine at the dns to form an alkylenedioxy group and
R¹⁰ is a phenyl or pyridyl group, which phenyl or pyridyl group is optionally substituted by 1 to 4 substituents selected from alkyl group optionally substituted by halogen atom, alkoxy group, hydroxyl group, halogen atom, cyano group, amino group and mono- or di-alkylamino group.

Another preferred embodiment includes compouds of the formula (I) wherein Ring A is a saturated or unsaturated 5- to 8-membered heterocyclic group containing 1 to 4 heteroatoms selected independently from oxygen, sulfur and nitrogen atoms, and
R¹¹ is a group selected from the following groups:
cyano group; nitro group; carboxyl group; sulfo group, alkyl group substituted by a group selected from halogen atom, hydroxyl group, carboxyl group, alkoxycarbonyl group, alkoxy group optionally substituted by phenyl or hydroxyl group, alkanoyloxy group, alkylthio group, alkylsulfonyl group, alkylsulfinyl group, amino group, mono- or di-alkylamino group, mono- or di-alkylsulfamoylamino group, mono- or dialkylureido group optionally substituted by morpholinyl group, oxiranyl group, di-alkyldioxolanyl group, pyrrolidinyl group optionally substituted by alkyl group, hexahydroazepinyl group and morpholinyl group; alkenyl group optionally substituted by carboxyl group or benzyloxycarbonyl group; alkoxy group substituted by carboxyl group, hydroxyl group, alkoxy group, alkylthio group, alkylsulfonyl group or alkoxyphenyl group; alkoxycarbonyl group optioally substituted by phenyl group, mono- or di-alkylcarbamoyl group optionally substituted by carboxyl group; hydroxycarbamimidoyl group; alkylthio group; alkylsulfinyl group; alkylsulfonyl group optionally substituted by alkoxycarbonyl group; mono- or di-alkylamino group optionally substituted by hydroxyl group, carboxyl group, alkoxycarbonyl group, alkoxy group, mono- or di-alkylamino group or morpholinyl group; mono- or di-alkylsulfamoyl group; morpholinyl group; optionally oxidized thiomorpholinyl group; piperazinyl group optionally substituted by a group selected from alkyl grop, alkanoyl group and hydroxyalkanoyl group; piperidinyl group optionally substituted by carboxyl group, alkyl group or alkoxycarbonyl group; dioxolanyl group optionally substituted by alkyl group; tetrazolyl group substituted by alkyl group, hydroxyalkl group or morpholinylalkyl group; dihydrooxadiazolyl group optionally substituted by oxo group; pyrimidinyl group; or tetrahydropyranyl group.

More preferred embodiment herein included compounds of the formula (I′) wherein R¹ is an alkoxycarbonyl group optionally substituted by a group selected from hydroxy group and alkoxy group; or a dihydrooxazolyl group optionally substituted by hydroxyalkyl group;
R² is an alkyl group;
R³ is a hydrogen atom;
R⁴ is an alkylene group;
Ring A and R¹¹ are the same groups as defined above;
R⁶ and R⁹ each are a hydrogen atom; and
R⁷ and R₈ are independently a hydrogen atom, an alkyl group optionally substituted by 1 to 9 halogen atoms, an alkoxy group, or a mono- or di-alkylamino group; or combine at the ends to form an alkylenedioxy group; R¹⁰ is a phenyl or pyridyl group, which phenyl or pyridyl group is optionally substituted by 1 to 4 substituents selected from alkyl group optionally substituted by 1 to 9 halogen atoms, alkoxy group, hydroxyl group, halogen atom, cyano group, amino group and mono- or di-alkylamino group.

Examples of Ring A include a pyrimidinyl group, a pyridyl group, a triazolyl group, a tetrazolyl group, an oxadiazolyl group, a dihydropyrimidinyl group, a pyrazinyl group, a thiazoyl group, an oxazolyl group, a dihydropyrazinyl group, and the like.

More preferred compounds include those where Ring A is a pyrimidinyl group, a pyridyl grop, a tetrazolyl group, an oxadiazolyl grop, a pyrazinyl group, a thiazolyl group or an oxazolyl group; and R¹¹ is a carboxyl group; a cyano group,; a nitro group; an alkyl group substituted by a group selected from hydroxyl group, cyano group, carboxyl group, alkoxycarbonyl group, alkoxy group, phenylalkoxy group, hydroxyalkoxy group, carboxyalkoxy group, alkylthio group, alkylsulfonyl group, alkylsulfinyl group, amino group, mono- or di-alkylamino group, mono- or di-alkylsulfamoylamino group, mono- or di-alkylureido group optionally substituted by morpholinyl group, oxiranyl group, di-alkyldioxolanyl group, pyrrolidinyl group optionally substituted by carboxyl group, piperidinyl group optionally substituted by carboxyl group, piperazinyl group optionally substituted by alkyl group and morpholinyl group; an alkenyl group optionally substituted by carboxyl group; an alkoxy group substituted by a group selected from hydroxyl group, cyano group, carboxyl group, alkoxycarbonyl group, alkoxy group, phenylalkoxy group, hydroxyalkoxy group, carboxyalkoxy group, alkylthio group, alkylsulfonyl group, alklsulfinyl group, amino group, mono- or dialkylamino group, mono- or di-alkylsulfamoylamino group, mono- or di-alkylamino group, mono- or di-alkylsulfamoylamino group, mono- or di-alkylureido group optionally substituted by morpholinyl group, oxiranyl group, di-alkyldioxolanyl group, pyrrolidinyl group optionally substituted by carboxyl group, piperazinyl group optionally substituted by alkyl group and morpholinyl group; an alkoxycarbonyl group; a hydroxycarbamimidoyl group; alkylthio group; an alkylsulfonyl group optionally substituted by carboxyl group; a mono- or di-alkylamino group optionally substituted by hydroxyl group; carboxyl group, alkoxy group or mono- or di-alkylamino group; a morpholinyl group; an optionally oxidized thiomorpholinyl group; a piperazinyl group optionally oxidized thiomorpholinyl group; a piperazinyl group optionally substituted by a group selected from alkyl group, alkanyoyl group and hydroxyalkanoyl group; a pyrrolidinyl group optionally substituted by carboxyl group, alkyl group carboxyalkyl group or alkoxcarbonyl group; a piperidinyl group optionally susbstituted by carboxyl group, alkyl group, carboxyalkyl group or alkoxycarbonyl group; a tetrazolyl group optionally substituted by alkyl group, hydroxyalkyl group, or morpholinylalkyl group; an oxodihydrooxadiazolyl group; a pyrimidinyl group; or a tetrahydropyranyl group.

Still more preferred compounds include those where R¹ is an alkoxycarbonyl group optionally substituted by 1 to 5 substituents selected independently from hydroxyl group, carboxyl group, alkoxycarbonyl group, alkoxy group (said alkoxy group is optionally substituted by 1 to 3 substituents selected independently from hydroxyl group, halogen atom, carboxyl group and alkoxycarbonyl group), alkenyl group, halogen atom, cycloalkyl group (said cycloalkyl group is optionally substituted by 1 to 3 substituents selected independently from hydroxy group, halogen atom, carboxyl group, alkoxycarbonyl group, carboxyalkyl group and alkoxycarbonylalkyl group), phenyl group (said phenyl group is optionally substituted by 1 to 3 substituents selected selected independently from hydroxyl group, halogen atom, carboxyl group, alkoxycarbonyl group, carboxyalkyl group and alkoxycarbonylalkyl group), piperidinyl group (said piperidinyl group is optionally substituted by 1 to 3 substituents selected independently from hydroxyl group, halogen atom, carboxyl group, alkoxycarbonyl group, carboxyalkyl group and alkoxycarbonylalkyl group), and pyrrolidinyl group (said pyrrolidinyl group is optionally substituted by 1 to 3 substituents selected independently from hydroxyl group, halogen atom, carboxyl group, alkoxycarbonyl group, carboxyalkyl group and alkoxcarbonylalkyl group); or a dihydrooxazolyl group optionally substituted by 1 or 2 substituents selected independently from carboxyl group, alkoxycarbonyl group, carboxyalkyl group, alkoxycarbonylalkyl group and hydroxyalkyl group;

R¹⁰ is a phenyl group substituted by 1 to 3 substituents selected from alkyl group optionally substituted by halogen atom, alkoxy group, halogen atom and cyano group;

Ring A is a pyrimidinyl group, a pyridl group, a tetrazolyl group, an oxadiazolyl group or a thiazolyl group; and

R¹¹ is a carboxyl group, a cyano group, a nitro group; and alkyl group substituted by a group selected from hydroxyl group, carboxyl group, alkoxycarbonyl group, alkoxy group, phenylalkoxy group, carboxyalkoxy group, alkylthio group, alkylsulfonyl group, alkylsulfinyl group, amino group, mono- or di-alkylaminio group, mono- or di-alkylsulfamoylamino group, mono- or di-alkylureido group optionally substituted by morpholinyl group, oxiranyl group, di-alkyldioxolanyl group, piperazinyl group optionally substituted by alkyl group and morpholinyl group; an alkenyl group optiojally substituted by carboxyl group; an alkoxy group substituted by a group selected from cyano group, hydroxyl group, carboxyl group, alkoxycarbonyl group, alkoxy grouop, phenylalkoxy group, carboxyalkoxy group, alkylthio group, alkylsulfonyl group, alkylsulfinyl group, amino group, mono- or di-alkylamino group, mono- or di-alkylsulfamoylamino group, mono- or di-alkyldioxolanyl group, piperazinyl group optionally substituted by alkyl group and morpholinyl group; a hydroxycarbamimidoyl group; an alkylthio group; an alkylsulfonyl group optionally substituted by alkoxycarbonyl group; a mono- or di-alkylamino group optionally substituted by hydroxyl group, carboxyl group or alkoxy group; a morpholinyl group; optionally oxidized thiomorpholinyl group, a piperazinyl group optionally substituted by a group selected from alkyl group, alkanoyl group and hydroxyalkanoyl group, a pyrrolidinyl group optionally substituted by carboxyl group, alkyl group carboxyalkyl group or alkoxycarbonyl group; a piperidinyl group optionally substituted by carboxyl group, alkyl group, carboxyalkyl group or alkoxycarbonyl group, a tetrazolyl group optionally substituted by alkyl group, hydroxyalkyl group, or mopholinylalkyl group; and oxodihydrooxadiazoyl group; a pyrimidinyl group; or a tetrahydropyranyl group.

Still furthermore preferred compounds include those where R¹ is an alkoxycarbonyl group optionally substituted by 1 or 5 substituents selected independently from carboxyl group, alkoxycarbonyl group, halogen atom, hydroxyl group and cycloalkyl group;
R¹⁰ is a phenyl group substituted by 1 to 3 substituents selected from cyano group, alkyl group optionally substituted by halogen atom and alkoxy group;
Ring A is a pyrimidinyl group, a pyridyl group, a tetrazolyl grop or an oxadiazolyl group;
R¹¹ is a carboxyl group; an alkyl group substituted by hydroxyl group, carboxyl group, alkoxy group or alkylsulfonyl group; an alkenyl group optionally substituted by carboxyl group; an alkoxy group substituted by cyano group, carboxyl group, hydroxyl group, alkoxy group, alkylthio group or alkylsulfonyl group; a mono- or di-alkylamino group optionally substituted by carboxyl group or alkoxy group; a morpholinyl group; a piperidinyl group substituted by hydroxyalkyl group;
R⁷ is an alkyl group optionally substituted by halogen atom, alkoxy grouop, or mono- or di-alkylamino group; and
R⁸ is a hydrogen atom.

Especially preferred compounds include those wherein R¹ is an ethoxycarbonyl group, a hydroxyethoxycarbonyl group, a 2-fluoroethoxycarbonyl group, a 2,2-difluoroethoxycarbonyl group or a 2,2,2-trifluoroethoxycarbonyl group; R² is an ethyl group; R¹⁰ is a phenyl group substituted by 1 to 2 substituents selected from cyano group, trifluoromethyl group and methoxy group; R⁷ is a trifluoromethyl group or a methoxy group. In this regard, other examples of especially preferred compounds include those wherein R¹ is a carboxy (C₂₋₁₀alkoxy) carbonyl group or an alkoxycarbonyl (C₂₋₁₀alkoxy) carbonyl group, and R²R¹⁰ and R⁷ are the same above.

Especially more preferred compounds include those wherein R¹ is an ethoxycarbonyl group or a hydroxyethoxycarbonyl group; R² is an ethyl group; R₁₀ is a phenyl group substituted by 1 to 2 substituents selected from cyano group, trifluoromethyl group and methoxy group; R⁷ is a trifluoromethyl group or a methoxy group.

Especially preferred sterol absorption inhibitors which may be combined in pharmaceutical combinations with compounds of Formula (I″) are those represented by Formula (I) 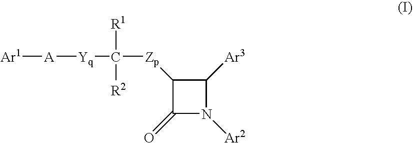
or isomers thereof, or pharmaceutically acceptable salts or solvates of the compounds of Formula (I) or of the isomers thereof, or prodrugs of the compounds of Formula (I) or of the isomers, salts or solvates thereof, wherein:

Ar¹ is R³-substituted aryl;

Ar² is R⁴-substituted aryl;

Ar³ is R⁵-substituted aryl;

Y and Z are independently selected from the group consisting of —CH₂—, —CH(lower alkyl)- and —C(dilower alkyl)-;

A is —O—, —S—, —S(O)— or —S(O)₂—;

R¹ is selected from the group consisting of —OR⁶, —O(CO)R⁶, —O(CO)OR⁹ and —O(CO)NR⁶R⁷;

R² is selected from the group consisting of hydrogen, lower alkyl and aryl; or R¹ and R² together are ═O;

q is 1, 2 or 3;

p is 0, 1, 2, 3 or 4;

R⁵ is 1-3 substituents independently selected from the group consisting of —OR⁶, —O(CO)R⁶, —O(CO)OR⁹, —O(CH₂)₁₋₅OR⁹, —O(CO)NR⁶R⁷, —NR⁶R⁷, —NR⁶(CO)R⁷, —NR⁶(CO)OR⁹, —NR⁶(CO)NR⁷R⁸, —NR⁶SO₂-lower alkyl, —NR⁶SO₂-aryl, —CONR⁶R⁷, —COR⁶, —SO₂NR⁶R⁷, S(O)₀₋₂-alkyl, S(O)₀₋₂-aryl, —O(CH₂)₁₋₁₀—COOR⁶, —O(CH₂)₁₋₁₀CONR⁶R⁷, o-halogeno, m-halogeno, o-lower alkyl, m-lower alkyl,

-(lower alkylene)-COOR⁶, and —CH═CH—COOR⁶;

R³ and R⁴ are independently 1-3 substituents independently selected from the group consisting of R⁵, hydrogen, p-lower alkyl, aryl, —NO₂, —CF₃ and p-halogeno;

R⁶, R⁷ and R⁸ are independently selected from the group consisting of hydrogen, lower alkyl, aryl and aryl-substituted lower alkyl; and

R⁹ is lower alkyl, aryl or aryl-substituted lower alkyl.

An especially preferred sterol absorption inhibitor which may be combined in pharmaceutical combinations with compounds of Formula (I″) is wherein the sterol absorption inhibitor is represented by Formula (VIII)

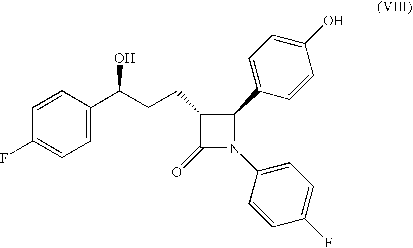

Generally, a total daily dosage of CETP inhibitor(s) can range from about 0.01 to about 1000 mg/day, and preferably about 0.5 to about 20 mg/kg body weight/day in single or divided doses.

In another alternative embodiment, the compositions or therapeutic combinations of the invention can further comprise probucol or derivatives thereof (such as AGI-1067 and other derivatives disclosed in U.S. Pat. Nos. 6,121,319 and 6,147,250), which can reduce LDL levels, coadministered with or in combination with the sterol absorption inhibitor(s) discussed above.

Generally, a total daily dosage of probucol or derivatives thereof can range from about 10 to about 2000 mg/day, and preferably about 500 to about 1500 mg/day in single or 2-4 divided doses.

In another alternative embodiment, the compositions or treatments of the invention can further comprise low-density lipoprotein (LDL) receptor activators, coadministered with or in combination with the sterol absorption inhibitor(s) discussed above. Non-limiting examples of suitable LDL-receptor activators include HOE-402, an imidazolidinyl-pyrimidine derivative that directly stimulates LDL receptor activity. See M. Hueftinger et al., “Hypolipidemic activity of HOE-402 is Mediated by Stimulation of the LDL Receptor Pathway”, Arterioscler. Thromb. 1993; 13:1005-12.

Generally, a total daily dosage of LDL receptor activator(s) can range from about 1 to about 1000 mg/day in single or 2-4 divided doses.

In another alternative embodiment, the compositions or therapeutic combinations of the invention can further comprise fish oil, which contains Omega 3 fatty acids (3-PUFA), which can reduce VLDL and triglyceride levels, coadministered with or in combination with sterol absorption inhibitor(s) discussed above. Generally, a total daily dosage of fish oil or Omega 3 fatty acids can range from about 1 to about 30 grams per day in single or 2-4 divided doses.

In another alternative embodiment, the compositions or therapeutic combinations of the invention can further comprise natural water soluble fibers, such as psyllium, guar, oat and pectin, which can reduce cholesterol levels, coadministered with or in combination with the sterol absorption inhibitor(s) discussed above. Generally, a total daily dosage of natural water soluble fibers can range from about 0.1 to about 10 grams per day in single or 24 divided doses.

In another alternative embodiment, the compositions or therapeutic combinations of the invention can further comprise plant sterols, plant stanols and/or fatty acid esters of plant stanols, such as sitostanol ester used in BENECOL® margarine, which can reduce cholesterol levels, coadministered with or in combination with the sterol absorption inhibitor(s) discussed above. Generally, a total daily dosage of plant sterols, plant stanols and/or fatty acid esters of plant stanols can range from about 0.5 to about 20 grams per day in single or 24 divided doses.

In another alternative embodiment, the compositions or therapeutic combinations of the invention can further comprise antioxidants, such as probucol, tocopherol, ascorbic acid, β-carotene and selenium, or vitamins such as vitamin B₆ or vitamin B₁₂, coadministered with or in combination with the sterol absorption inhibitor(s) discussed above. Generally, a total daily dosage of antioxidants or vitamins can range from about 0.05 to about 10 grams per day in single or 24 divided doses.

In another alternative embodiment, the compositions or therapeutic combinations of the invention can further comprise monocyte and macrophage inhibitors such as polyunsaturated fatty acids (PUFA), thyroid hormones including throxine analogues such as CGS-26214 (a thyroxine compound with a fluorinated ring), gene therapy and use of recombinant proteins such as recombinant apo E, coadministered with or in combination with the sterol absorption inhibitor(s) discussed above. Generally, a total daily dosage of these agents can range from about 0.01 to about 1000 mg/day in single or 2-4 divided doses.

Also useful with the invention are compositions or therapeutic combinations which further comprise hormone replacement agents and compositions. Useful hormone agents and compositions for hormone replacement therapy of the present invention include androgens, estrogens, progestins, their pharmaceutically acceptable salts and derivatives thereof. Combinations of these agents and compositions are also useful.

The dosage of androgen and estrogen combinations vary, desirably from about 1 mg to about 4 mg androgen and from about 1 mg to about 3 mg estrogen. Examples include, but are not limited to, androgen and estrogen combinations such as the combination of esterified estrogens (sodium estrone sulfate and sodium equilin sulfate) and methyltestosterone (17-hydroxy-17-methyl-, (17B)- androst-4-en-3-one) available from Solvay Pharmaceuticals, Inc., Marietta, Ga., under the tradename Estratest.

Estrogens and estrogen combinations may vary in dosage from about 0.01 mg up to 8 mg, desirably from about 0.3 mg to about 3.0 mg. Examples of useful estrogens and estrogen combinations include:

(a) the blend of nine (9) synthetic estrogenic substances including sodium estrone sulfate, sodium equilin sulfate, sodium 17 α-dihydroequilin sulfate, sodium 17 α-estradiol sulfate, sodium 17, β-dihydroequilin sulfate, sodium 17 α-dihydroequilenin sulfate, sodium 17 β-dihydroequilenin sulfate, sodium equilenin sulfate and sodium 17 β-estradiol sulfate; available from Duramed Pharmaceuticals, Inc., Cincinnati, Ohio, under the tradename Cenestin;

(b) ethinyl estradiol (19-nor-17 α-pregna-1,3,5(110)-trien-20-yne-3,17-diol; available by Schering Plough Corporation, Kenilworth, N.J., under the tradename Estinyl;

(c) esterified estrogen combinations such as sodium estrone sulfate and sodium equilin sulfate; available from Solvay under the tradename Estratab and from Monarch Pharmaceuticals, Bristol, Tenn., under the tradename Menest;

(d) estropipate (piperazine estra-1,3,5(10)-trien-17-one, 3-(sulfooxy)-estrone sulfate); available from Pharmacia & Upjohn, Peapack, N.J., under the tradename Ogen and from Women First Health Care, Inc., San Diego, Calif., under the tradename Ortho-Est; and

(e) conjugated estrogens (17 α-dihydroequilin, 17 α-estradiol, and 17β-dihydroequilin); available from Wyeth-Ayerst Pharmaceuticals, Philadelphia, Pa., under the tradename Premarin.

Progestins and estrogens may also be administered with a variety of dosages, generally from about 0.05 to about 2.0 mg progestin and about 0.001 mg to about 2 mg estrogen, desirably from about 0.1 mg to about 1 mg progestin and about 0.01 mg to about 0.5 mg estrogen. Examples of progestin and estrogen combinations that may vary in dosage and regimen include:

(a) the combination of estradiol (estra-1,3,5 (10)-triene-3,17 β-diol hemihydrate) and norethindrone (17β-acetoxy-19-nor-17 α-pregn-4-en-20-yn-3-one); which is available from Pharmacia & Upjohn, Peapack, N.J., under the tradename Activella;

(b) the combination of levonorgestrel (d(−)-13β-ethyl-17α-ethinyl-17β-hydroxygon-4-en-3-one) and ethinyl estradial; available from Wyeth-Ayerst under the tradename Alesse, from Watson Laboratories, Inc., Corona, Calif., under the tradenames Levora and Trivora, Monarch Pharmaceuticals, under the tradename Nordette, and from Wyeth-Ayerst under the tradename Triphasil;

(c) the combination of ethynodiol diacetate (19-nor-17α-pregn-4-en-20-yne-3α, 17-diol diacetate) and ethinyl estradiol; available from G.D. Searle & Co., Chicago, Ill., under the tradename Demulen and from Watson under the tradename Zovia;

(d) the combination of desogestrel (13-ethyl-11-methylene-18,19-dinor-17 α-pregn-4-en-20-yn-17-ol) and ethinyl estradiol; available from Organon under the tradenames Desogen and Mircette, and from Ortho-McNeil Pharmaceutical, Raritan, N.J., under the tradename Ortho-Cept;

(e) the combination of norethindrone and ethinyl estradiol; available from Parke-Davis, Morris Plains, N.J., under the tradenames Estrostep and femhrt, from Watson under the tradenames Microgestin, Necon, and Tri-Norinyl, from Ortho-McNeil under the tradenames Modicon and Ortho-Novum, and from Warner Chilcott Laboratories, Rockaway, N.J., under the tradename Ovcon;

(f) the combination of norgestrel ((±)-13-ethyl-17-hydroxy-18,19-dinor-17 α-preg-4-en-20-yn-3-one) and ethinyl estradiol; available from Wyeth-Ayerst under the tradenames Ovral and Lo/Ovral, and from Watson under the tradenames Ogestrel and Low-Ogestrel;

(g) the combination of norethindrone, ethinyl estradiol, and mestranol (3-methoxy-19-nor-17α-pregna-1,3,5(10)-trien-20-yn-17-ol); available from Watson under the tradenames Brevicon and Norinyl;

(h) the combination of 17β-estradiol (estra-1,3,5(10)-triene-3,17β-diol) and micronized norgestimate (17α-17-(Acetyloxyl)-13-ethyl-18,19-dinorpregn-4-en-20-yn-3-one3-oxime); available from Ortho-McNeil under the tradename Ortho-Prefest;

(i) the combination of norgestimate (18,19-dinor-17-pregn-4-en-20-yn-3-one, 17-(acetyloxy)-13-ethyl-, oxime, (17(α)-(+)−) and ethinyl estradiol; available from Ortho-McNeil under the tradenames Ortho Cyclen and Ortho Tri-Cyclen; and

(j) the combination of conjugated estrogens (sodium estrone sulfate and sodium equilin sulfate) and medroxyprogesterone acetate (20-dione, 17-(acetyloxy)-6-methyl-, (6(α))-pregn-4-ene-3); available from Wyeth-Ayerst under the tradenames Premphase and Prempro.

In general, a dosage of progestins may vary from about 0.05 mg to about 10 mg or up to about 200 mg if microsized progesterone is administered. Examples of progestins include norethindrone; available from ESI Lederle, Inc., Philadelphia, Pa., under the tradename Aygestin, from Ortho-McNeil under the tradename Micronor, and from Watson under the tradename Nor-QD; norgestrel; available from Wyeth-Ayerst under the tradename Ovrette; micronized progesterone (pregn-4-ene-3,20-dione); available from Solvay under the tradename Prometrium; and medroxyprogesterone acetate; available from Pharmacia & Upjohn under the tradename Provera.

The compositions, therapeutic combinations or methods of the invention can further comprise one or more obesity control medications. Useful obesity control medications include, but are not limited to, drugs that reduce energy intake or suppress appetite, drugs that increase energy expenditure and nutrient-partitioning agents. Suitable obesity control medications include, but are not limited to, noradrenergic agents (such as diethylpropion, mazindol, phenylpropanolamine, phentermine, phendimetrazine, phendamine tartrate, methamphetamine, phendimetrazine and tartrate); serotonergic agents (such as sibutramine, fenfluramine, dexfenfluramine, fluoxetine, fluvoxamine and paroxtine); thermogenic agents (such as ephedrine, caffeine, theophylline, and selective, β3-adrenergic agonists); alpha-blocking agents; kainite or AMPA receptor antagonists; leptin-lipolysis stimulated receptors; phosphodiesterase enzyme inhibitors; compounds having nucleotide sequences of the mahogany gene; fibroblast growth factor-10 polypeptides; monoamine oxidase inhibitors (such as befloxatone, moclobemide, brofaromine, phenoxathine, esuprone, befol, toloxatone, pirlindol, amiflamine, sercloremine, bazinaprine, lazabemide, milacemide and caroxazone); compounds for increasing lipid metabolism (such as evodiamine compounds); and lipase inhibitors (such as orlistat). Generally, a total dosage of the above-described obesity control medications can range from 1 to 3,000 mg/day, desirably from about 1 to 1,000 mg/day and more desirably from about 1 to 200 mg/day in single or 2-4 divided doses.

The compositions, therapeutic combinations or methods of the invention can further comprise one or more blood modifiers which are chemically different from the substituted azetidinone and substituted β-lactam compounds discussed above. Useful blood modifiers include but are not limited to anti-coagulants (argatroban, bivalirudin, dalteparin sodium, desirudin, dicumarol, lyapolate sodium, nafamostat mesylate, phenprocoumon, tinzaparin sodium, warfarin sodium); antithrombotic (anagrelide hydrochloride, bivalirudin, cilostazol, dalteparin sodium, danaparoid sodium, dazoxiben hydrochloride, efegatran sulfate, enoxaparin sodium, fluretofen, ifetroban, ifetroban sodium, lamifiban, lotrafiban hydrochloride, napsagatran, orbofiban acetate, roxifiban acetate, sibrafiban, tinzaparin sodium, trifenagrel, abciximab, zolimomab aritox); fibrinogen receptor antagonists (roxifiban acetate, fradafiban, orbofiban, lotrafiban hydrochloride, tirofiban, xemilofiban, monoclonal antibody 7E3, sibrafiban); platelet inhibitors (cilostazol, clopidogrel bisulfate, epoprostenol, epoprostenol sodium, ticlopidine hydrochloride, aspirin, ibuprofen, naproxen, sulindae, idomethacin, mefenamate, droxicam, diclofenac, sulfinpyrazone, piroxicam, dipyridamole); platelet aggregation inhibitors (acadesine, beraprost, beraprost sodium, ciprostene calcium, itazigrel, lifarizine, lotrafiban hydrochloride, orbofiban acetate, oxagrelate, fradafiban, orbofiban, tirofiban, xemilofiban); hemorrheologic agents (pentoxifylline); lipoprotein associated coagulation inhibitors; Factor VIIa inhibitors (4H-31-benzoxazin-4-ones, 4H-3,1-benzoxazin-4-thiones, quinazolin-4-ones, quinazolin-4-thiones, benzothiazin-4-ones, imidazolyl-boronic acid-derived peptide analogues TFPI-derived peptides, naphthalene-2-sulfonic acid {1-[3-(aminoiminomethyl)-benzyl]-2-oxo-pyrrolidin-3-(S)-yl}amide trifluoroacetate, dibenzofuran-2-sulfonic acid {1-[3-(aminomethyl)-benzyl]-5-oxo-pyrrolidin-3-yl}-amide, tolulene-4-sulfonic acid {1-[3-(aminoiminomethyl)-benzyl]-2-oxo-pyrrolidin-3-(S)-yl}-amide trifluoroacetate, 3,4-dihydro-1H-isoquinoline-2-sulfonic acid {1-[3-(aminoiminomethyl)-benzyl]-2-oxo-pyrrolin-3-(S)-yl}-amide trifluoroacetate); Factor Xa inhibitors (disubstituted pyrazolines, disubstituted triazolines, substituted n-[(aminoiminomethyl)phenyl]propylamides, substituted n-[(aminomethyl)phenyl]propylamides, tissue factor pathway inhibitor (TFPI), low molecular weight heparins, heparinoids, benzimidazolines, benzoxazolinones, benzopiperazinones, indanones, dibasic (amidinoaryl) propanoic acid derivatives, amidinophenyl-pyrrolidines, amidinophenyl-pyrrolines, amidinophenyl-isoxazolidines, amidinoindoles, amidinoazoles, bis-arlysulfonylaminobenzamide derivatives, peptidic Factor Xa inhibitors).

The compositions, therapeutic combinations or methods of the invention can further comprise one or more cardiovascular agents which are chemically different from the substituted azetidinone and substituted β-lactam compounds (such as compounds I-XI above) discussed above. Useful cardiovascular agents include but are not limited to calcium channel blockers (clentiazem maleate, amlodipine besylate, isradipine, nimodipine, felodipine, nilvadipine, nifedipine, teludipine hydrochloride, diltiazem hydrochloride, belfosdil, verapamil hydrochloride, fostedil); adrenergic blockers (fenspiride hydrochloride, labetalol hydrochloride, proroxan, alfuzosin hydrochloride, acebutolol, acebutolol hydrochloride, alprenolol hydrochloride, atenolol, bunolol hydrochloride, carteolol hydrochloride, celiprolol hydrochloride, cetamolol hydrochloride, cicloprolol hydrochloride, dexpropranolol hydrochloride, diacetolol hydrochloride, dilevalol hydrochloride, esmolol hydrochloride, exaprolol hydrochloride, flestolol sulfate, labetalol hydrochloride, levobetaxolol hydrochloride, levobunolol hydrochloride, metalol hydrochloride, metoprolol, metoprolol tartrate, nadolol, pamatolol sulfate, penbutolol sulfate, practolol, propranolol hydrochloride, sotalol hydrochloride, timolol, timolol maleate, tiprenolol hydrochloride, tolamolol, bisoprolol, bisoprolol fumarate, nebivolol); adrenergic stimulants; angiotensin converting enzyme (ACE) inhibitors (benazepril hydrochloride, benazeprilat, captopril, delapril hydrochloride, fosinopril sodium, libenzapril, moexipril hydrochloride, pentopril, perindopril, quinapril hydrochloride, quinaprilat, ramipril, spirapril hydrochloride, spiraprilat, teprotide, enalapril maleate, lisinopril, zofenopril calcium, perindopril erbumine); antihypertensive agents (althiazide, benzthiazide, captopril, carvedilol, chlorothiazide sodium, clonidine hydrochloride, cyclothiazide, delapril hydrochloride, dilevalol hydrochloride, doxazosin mesylate, fosinopril sodium, guanfacine hydrochloride, methyldopa, metoprolol succinate, moexipril hydrochloride, monatepil maleate, pelanserin hydrochloride, phenoxybenzamine hydrochloride, prazosin hydrochloride, primidolol, quinapril hydrochloride, quinaprilat, ramipril, terazosin hydrochloride, candesartan, candesartan cilexetil, telmisartan, amlodipine besylate, amlodipine maleate, bevantolol hydrochloride); angiotensin II receptor antagonists (candesartan, irbesartan, losartan potassium, candesartan cilexetil, telmisartan); anti-anginal agents (amlodipine besylate, amlodipine maleate, betaxolol hydrochloride, bevantolol hydrochloride, butoprozine hydrochloride, carvedilol, cinepazet maleate, metoprolol succinate, molsidomine, monatepil maleate, primidolol, ranolazine hydrochoride, tosifen, verapamil hydrochloride); coronary vasodilators (fostedil, azaclorzine hydrochloride, chromonar hydrochloride, clonitrate, diltiazem hydrochloride, dipyridamole, droprenilamine, erythrityl tetranitrate, isosorbide dinitrate, isosorbide mononitrate, lidoflazine, mioflazine hydrochloride, mixidine, molsidomine, nicorandil, nifedipine, nisoldipine, nitroglycerine, oxprenolol hydrochloride, pentrinitrol, perhexiline maleate, prenylamine, propatyl nitrate, terodiline hydrochloride, tolamolol, verapamil); diuretics (the combination product of hydrochlorothiazide and spironolactone and the combination product of hydrochlorothiazide and triamterene).

The compositions, therapeutic combinations or methods of the invention can further comprise one or more antidiabetic medications for reducing blood glucose levels in a human. Useful antidiabetic medications include, but are not limited to, drugs that reduce energy intake or suppress appetite, drugs that increase energy expenditure and nutrient-partitioning agents. Suitable antidiabetic medications include, but are not limited to, sulfonylurea (such as acetohexamide, chlorpropamide, gliamilide, gliclazide, glimepiride, glipizide, glyburide, glibenclamide, tolazamide, and tolbutamide), meglitinide (such as repaglinide and nateglinide), biguanide (such as metformin and buformin), alpha-glucosidase inhibitor (such as acarbose, miglitol, camiglibose, and voglibose), certain peptides (such as amlintide, pramlintide, exendin, and GLP-1 agonistic peptides), and orally administrable insulin or insulin composition for intestinal delivery thereof. Generally, a total dosage of the above-described antidiabetic medications can range from 0.1 to 1,000 mg/day in single or 24 divided doses.

Mixtures of any of the pharmacological or therapeutic agents described above can be used in the compositions and therapeutic combinations of the invention.

The treatment compositions of the invention generally additionally comprise a pharmaceutically acceptable carrier diluent, excipient or carrier (collectively referred to herein as carrier materials). Because of their sterol absorption inhibitory activity, such pharmaceutical compositions possess utility in treating sitosterolemia and related disorders.

In the treatment compositions used in the methods of the present invention, the active ingredients will typically be administered in admixture with suitable carrier materials suitably selected with respect to the intended form of administration, i.e. oral tablets, capsules (either solid-filled, semi-solid filled or liquid filled), powders for constitution, oral gels, elixirs, dispersible granules, syrups, suspensions, and the like, and consistent with conventional pharmaceutical practices. For example, for oral administration in the form of tablets or capsules, the active drug component may be combined with any oral non-toxic pharmaceutically acceptable inert carrier, such as lactose, starch, sucrose, cellulose, magnesium stearate, dicalcium phosphate, calcium sulfate, talc, mannitol, ethyl alcohol (liquid forms) and the like. Moreover, when desired or needed, suitable binders, lubricants, disintegrating agents and coloring agents may also be incorporated in the mixture. Powders and tablets may be comprised of from about 5 to about 95 percent inventive composition.

Suitable binders include starch, gelatin, natural sugars, corn sweeteners, natural and synthetic gums such as acacia, sodium alginate, carboxymethyl-cellulose, polyethylene glycol and waxes. Among the lubricants there may be mentioned for use in these dosage forms, boric acid, sodium benzoate, sodium acetate, sodium chloride, and the like. Disintegrants include starch, methylcellulose, guar gum and the like. Sweetening and flavoring agents and preservatives may also be included where appropriate. Some of the terms noted above, namely disintegrants, diluents, lubricants, binders and the like, are discussed in more detail below.

Additionally, the compositions of the present invention may be formulated in sustained release form to provide the rate controlled release of any one or more of the components or active ingredients to optimize the therapeutic effects, i.e. sterol absorption inhibitory activity and the like. Suitable dosage forms for sustained release include layered tablets containing layers of varying disintegration rates or controlled release polymeric matrices impregnated with the active components and shaped in tablet form or capsules containing such impregnated or encapsulated porous polymeric matrices.

Liquid form preparations include solutions, suspensions and emulsions. As an example may be mentioned water or water-propylene glycol solutions for parenteral injections or addition of sweeteners and pacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier such as inert compressed gas, e.g. nitrogen.

For preparing suppositories, a low melting wax such as a mixture of fatty acid glycerides such as cocoa butter is first melted, and the active ingredient is dispersed homogeneously therein by stirring or similar mixing. The molten homogeneous mixture is then poured into convenient sized molds, allowed to cool and thereby solidify.

Also included are solid form preparations which are intended to be converted, shortly before use, to liquid form preparations for either oral or parenteral administration. Such liquid forms include solutions, suspensions and emulsions.

The compounds of the invention may also be deliverable transdermally. The transdermal compositions may take the form of creams, lotions, aerosols and/or emulsions and can be included in a transdermal patch of the matrix or reservoir type as are conventional in the art for this purpose.

Preferably the compound is administered orally, intravenously or subcutaneously.

Preferably, the pharmaceutical preparation is in a unit dosage form. In such form, the preparation is subdivided into suitably sized unit doses containing appropriate quantities of the active components, e.g., an effective amount to achieve the desired purpose.

The pharmaceutical treatment compositions of the present invention can be administered to a mammal in need of such treatment in a pharmaceutically effective amount to treat sitosterolemia and/or reduce the level of sterol(s) in the plasma and tissues. The daily dose of the sterol absorption inhibitor(s) preferably ranges from about 0.1 to about 30 mg/kg of body weight per day, and more preferably about 0.1 to about 15 mg/kg. For an average body weight of 70 kg, the dosage level therefore ranges from about 1 mg to about 1000 mg of sterol absorption inhibitor(s) per day, given in a single dose or 2-4 divided doses. The exact dose, however, is determined by the attending clinician and is dependent on the potency of the compound administered, the age, weight, condition and response of the patient.

For the pharmaceutical treatment compositions of the present invention in which the sterol absorption inhibitor(s) is administered concomitantly or in combination with a bile acid sequestrant, the typical daily dose of the sequestrant preferably ranges from about 0.1 to about 80 mg/kg of body weight per day administered in single or divided dosages, usually once or twice a day. For example, preferably about 10 to about 40 mg per dose is given 1 to 2 times a day, giving a total daily dose of about 10 to about 80 mg per day. The exact dose of sterol absorption inhibitor(s) and bile acid sequestrant(s) to be administered is determined by the attending clinician and is dependent on the potency of the compound administered, the age, weight, condition and response of the patient.

Where the sterol absorption inhibitor(s) and bile acid sequestrant(s) are administered in separate dosages, the number of doses of each component given per day may not necessarily be the same, e.g., one component may have a greater duration of activity and will therefore need to be administered less frequently.

For the pharmaceutical treatment compositions of the present invention in which the sterol absorption inhibitor(s) is administered concomitantly or in combination with a lipid lowering agent, the typical daily dose of the lipid lowering agent preferably ranges from about 0.1 to about 80 mg/kg of body weight per day administered in single or divided dosages, usually once or twice a day. For example, for HMG CoA reductase inhibitors, preferably about 10 to about 40 mg per dose is given 1 to 2 times a day, giving a total daily dose of about 10 to about 80 mg per day. For other lipid lowering agents, preferably about 1 to about 1000 mg per dose is given 1 to 2 times a day, giving a total daily dose ranging from about 1 mg to about 2000 mg per day. The exact dose of sterol absorption inhibitor(s) and lipid lowering agent(s) to be administered is determined by the attending clinician and is dependent on the potency of the compound administered, the age, weight, condition and response of the patient.

Where the sterol absorption inhibitor(s) and lipid lowering agent(s) are administered in separate dosages, the number of closes of each component given per day may not necessarily be the same, e.g., one component may have a greater duration of activity and will therefore need to be administered less frequently.

The formulations and pharmaceutical compositions can be prepared using conventional pharmaceutically acceptable and conventional techniques. The following formulations exemplify some of the dosage forms of this invention. In each formulation, the term “active compound” designates a substituted azetidinone compound, a β-lactam compound or a compound of any of the Formulas I-XI described herein above.

### EXAMPLE A

Tablets

| No. | Ingredient | mg/tablet | mg/tablet |
|---|---|---|---|
| 1 | Active Compound | 100 | 500 |
| 2 | Lactose USP | 122 | 113 |
| 3 | Corn Starch, Food Grade, as a 10% | 30 | 40 |
| | paste in Purified Water | | |
| 4 | Corn Starch, Food Grade | 45 | 40 |
| 5 | Magnesium Stearate | 3 | 7 |
| | Total | 300 | 700 |

Method of Manufacture

Mix Item Nos. 1 and 2 in suitable mixer for 10-15 minutes. Granulate the mixture with Item No. 3. Mill the damp granules through a coarse screen (e.g., ¼″, 0.63 cm) if necessary. Dry the damp granules. Screen the dried granules if necessary and mix with Item No. 4 and mix for 10-15 minutes. Add Item No. 5 and mix for 1-3 minutes. Compress the mixture to appropriate size and weight on a suitable tablet machine.

### EXAMPLE B

Capsules

| No. | Ingredient | mg/tablet | mg/tablet |
|---|---|---|---|
| 1 | Active Compound | 100 | 500 |
| 2 | Lactose USP | 106 | 123 |
| 3 | Corn Starch, Food Grade | 40 | 70 |
| 4 | Magnesium Stearate NF | 4 | 7 |
| | Total | 250 | 700 |

Method of Manufacture

Mix Item Nos. 1, 2 and 3 in a suitable blender for 10-15 minutes. Add Item No. 4 and mix for 1-3 minutes. Fill the mixture into suitable two-piece hard gelatin capsules on a suitable encapsulating machine.

### EXAMPLE C

Tablets

| No. | Ingredient | mg/tablet |
|---|---|---|
| 1 | Active Compound I | 10 |
| 2 | Lactose monohydrate NF | 55 |
| 3 | Microcrystalline cellulose NF | 20 |
| 4 | Povidone (K29-32) USP | 4 |
| 5 | Croscarmellose sodium NF | 8 |
| 6 | Sodium lauryl sulfate | 2 |
| 7 | Magnesium stearate NF | 1 |
| | Total | 100 |

Method of Manufacture

Mix Item No. 4 with purified water in suitable mixer to form binder solution. Spray the binder solution and then water over Items 1, 2, 6 and a portion of Item 5 in a fluidized bed processor to granulate the ingredients. Continue fluidization to dry the damp granules. Screen the dried granules and blend with Item No. 3 and the remainder of Item 5. Add Item No. 7 and mix. Compress the mixture to appropriate size and weight on a suitable tablet machine.

In the present invention, the above-described tablet can be coadministered with a tablet, capsule, etc. comprising a dosage of another therapeutic agent such as are described above, for example a bile acid sequestrant as described above.

Representative formulations comprising other lipid lowering agents are well known in the art. It is contemplated that where the two active ingredients are administered as a single composition, the dosage forms disclosed above for substituted azetidinone compounds may readily be modified using the knowledge of one skilled in the art.

The treatment compositions of the present invention can inhibit the intestinal absorption of sitosterol in an animal model, as shown in the Example below. Thus, the treatment compositions of the present invention are hypositosterolemic agents by virtue of their ability to inhibit the intestinal absorption of sitosterol and can be useful in the treatment and/or prevention of atherosclerosis and sitosterolemia in mammals, in particular in humans.

In another embodiment of the invention, the compositions and therapeutic combinations of the present invention can reduce plasma concentration of at least one sterol selected from the group consisting of phytosterols (such as sitosterol, campesterol, stigmasterol and avenosterol), 5α-stanols (such as cholestanol, 5α-campestanol, 5α-sitostanol), cholesterol and mixtures thereof. The plasma concentration can be reduced by administering to a mammal in need of such treatment an effective amount of at least one treatment composition or therapeutic combination comprising at least one sterol absorption inhibitor described above. The reduction in plasma concentration of sterols can range from about 1 to about 70 percent, and preferably about 10 to about 50 percent. Methods of measuring serum total blood cholesterol and total LDL cholesterol are well known to those skilled in the art and for example include those disclosed in PCT WO 99/38498 at page 11, incorporated by reference herein. Methods of determining levels of other sterols in serum are disclosed in H. Gylling et al., “Serum Sterols During Stanol Ester Feeding in a Mildly Hypercholesterolemic Population”, J. Lipid Res. 40: 593-600 (1999), incorporated by reference herein.

In an alternative embodiment, the plasma and tissue concentration of sterols can be reduced by administering to a mammal in need of such treatment an effective amount of at least one treatment composition comprising at least one sterol absorption inhibitor and an effective amount of at least one bile acid sequestrant.

In a further embodiment, the plasma and tissue concentration of sterols can be reduced by administering to a mammal in need of such treatment an effective amount of at least one treatment composition comprising at least one sterol absorption inhibitor and an effective amount of at least one other lipid lowering agent.

Illustrating the invention are the following examples which, however, are not to be considered as limiting the invention the their details. Unless indicated otherwise, all parts and percentages in the following examples, as well as throughout the specification, are by weight.

### EXAMPLE 1

### In Vivo Evaluation in Mice

In vivo activity of compound VIII in mice was determined by the following procedure:

Male ApoE knockout mice, age 6 wks, were received from Jackson Laboratory along with age-matched C57BL/J. The mice were housed 5 per cage, normal light cycle, normal diet. Twenty-six mice of each variety were weighed and housed, 1 per cage, in suspended wire cages with normal light cycle, normal diet. After three days, the mice were reweighed. Based on body weight, the mice were divided into 5 groups for each type of treatment:

Control (corn oil) and Compositions including Compound VIII at 0.3, 1, 3, and 10 mg/kg of body weight per day.

Preparation of Compositions including Compound VIII based on 22 g average mouse body weight:

| Dosage of Compound | Compound VIII (ml) + |
|---|---|
| VIII (mg/ml/day) | corn oil (ml) |
| 10 mg/kg/day in 0.1 ml | 2.2 mg/ml * 10 ml = 22 mg in 10 ml corn oil |
| corn oil | |
| 3 mg/kg: | 3 ml of 10 mg/kg + 7 ml corn oil; |
| 1 mg/kg: | 3 ml of 3 mg/kg + 6 ml corn oil; |
| 0.3 mg/kg: | 2 ml of 1 mg/kg + 4.67 ml corn oil. |

The mice were gavaged using a feeding needle 30 min before receiving ¹⁴C-cholesterol (NEN, NEC 018) and ³H-sitosterol (NEN, CUS 030T). The radioactive dose was prepared from:

114 μL ³H-sitosterol stock (1 μCi/μL in ethanol);

1.425 mL ¹⁴C-cholesterol stock (40 μCi/mL in ethanol);

5.7 mg cholesterol, Sigma C 8667;

5.7 mg β-sitosterol, Sigma, S 1270;

The ethanol was removed under N₂;

5.7 ml of corn oil was added, and the mixture was warmed to 60° C.; and shaken for 1 hr.

Each 0.1 ml dose contained 2 μCi ³H-sitosterol, 0.1 mg cold (non radioactive) sitosterol; 1 μCi ¹⁴C-cholesterol, and 0.1 mg cold (non radioactive) cholesterol. Radioactive content was verified: 5×10 μl counted in Beckman LSC (liquid simulation counter). Tritiated sitosterol was used as an “unabsorbable” marker to compare to the absorption of [¹⁴C]-cholesterol in a mouse fecal isotope ratio cholesterol absorption model.

On the 4^{th}, 5^{th}, and 6^{th} days, feces were collected and stored at −20° C. in vials just before dosing with Control or Compound VIII late in the day. Termination of the experiment on the 7^{th} day involved sacrifice by exsanguination, removal and weighing of the liver. 3×˜250 mg samples of liver were put in vials. The liver samples were digested with 1 ml of 1N NaOH at 60° overnight, neutralized with 0.1 ml 12N HCl and counted for ¹⁴C and ³H. The blood samples were allowed to clot at room temp for 1 hr, then centrifuged at 1000G for 15 min. The serum was analyzed for total cholesterol (see Wako CII; see Allain C C, Poon L S, Chan C S G, Richmond W, Fu P C. Enzymatic Determination of Total Serum Cholesterol. Clin. Chem. 1974; 20:470-475, which is incorporated by reference herein) and radioactivity (2×50 μL). Fecal samples were analyzed for radioactivity by combustion in a Packard Oxidizer followed by Beckman L S C.

In this experiment, Wild type mice (C57BL/6J) and mice deficient in apoprotein E (Apo E KO) were found to absorb from 0.15-0.38% of the original [³H]-sitosterol dose administered into their livers. When Compound VIII was given, it was found to dose dependently inhibit the absorption and hepatic accumulation of sitosterol as shown in Table 1 below.

TABLE 1
Effect of Compound VIII on Sitosterol Absorption in Mice

| | |
|---|---|
| | % of administered dose |
| | absorbed of [³H]- |
| | sitosterol in liver |
| Mouse | (total animal liver) |

| strain | Treatment | average ± sem | p = |
|---|---|---|---|
| C57BL/6J | Control | 0.1479 ± 0.0337 | |
| | Compound VIII | 0.1093 ± 0.0143 | |
| | 0.3 mg/kg | | |
| | Compound VIII | 0.0588 ± 0.0115 | (.046) |
| | 1 mg/kg | | |
| | Compound VIII | 0.0489 ± 0.0067 | (.024) |
| | 3 mg/kg | | |
| | Compound VIII | 0.0552 ± 0.0151 | (.040) |
| | 10 mg/kg | | |
| ApoE KO | Control | 0.3773 ± 0.0525 | |
| | Compound VIII | 0.1863 ± 0.0246 | 0.013 |
| | 0.3 mg/kg | | |
| | Compound VIII | 0.1019 ± 0.0225 | 0.0019 |
| | 1 mg/kg | | |
| | Compound VIII | 0.0772 ± 0.0050 | 0.0023 |
| | 3 mg/kg | | |
| | Compound VIII | 0.0780 ± 0.0179 | 0.0017 |
| | 10 mg/kg | | |

N = 4-6 mice per treatment
sem = standard error of mean
p = probability

### EXAMPLE 2

### In Vivo Evaluation in Humans

In a randomized multicenter, double-blind, placebo-controlled, 8-week trial, 37 human patients previously diagnosed with homozygous sitosterolemia were randomized to receive Compound VIII (n=30) or placebo (n=7):

Treatment A—Compound VIII given orally as 1 dose (10 mg) per day,

Treatment B—Placebo (matching image of Compound VIII 10 mg) given orally as 1 dose per day, every morning for 8 consecutive weeks.

During the trial, subjects were instructed to maintain (as a minimum) a National Cholesterol Education Program (NCEP) Step 1 diet

Patients were instructed to maintain a diary of food intake and monitored prior to randomization, at baseline and during therapy. Results of the central diet analysis for each subject were reported as a RISCC score (Ratio of Ingested Saturated fat and Cholesterol to Calories) and as dietary components. RISCC scores indicate the potential for a diet to influence plasma lipid levels. A score ranging from 14 to 20 correlates with a NCEP step 1 diet.

Lipid/Lipoproteins Determinations

Low-Density-Lipoprotein-Cholesterol (LDL-C) results were reported as direct LDL-C (plasma concentration was determined following a standard ultra centrifugation/precipitation procedure; lipid and lipoprotein analysis, see Manual of Laboratory Operations: Lipid Research Clinics Program Report. Washington, D.C.: US Department of Health, Education, and Welfare publication; 1974. NIH 75-628, vol 1, which is incorporated by reference herein or beta-quantification) and calculated LDL-C (plasma concentration; based on Freidewald equation: LDL-C=Total cholesterol minus (Triglycerides divided by 5) minus High-density-lipoprotein cholesterol (HDL-C)).

Total cholesterol and Triglycerides were determined enzymatically using a Hitachi 747 analyzer; see, Steiner P M, Freidel J, Bremner W F, Stein E A: Standardization of micromethods for plasma cholesterol, triglyceride and HDL-cholesterol with the Lipid Clinics' methodology [abstract]. J Clin Chem Clin Biochem 1981; 19:850, which is incorporated by reference herein.

HDL-C was determined enzymatically after heparin and magnesium precipitation; see, Steele W B, Koehle D F, Azar M M, Blaszkowski T P, Kuba K, Dempsey M E: Enzymatic determinations of cholesterol in high density lipoprotein fractions prepared by precipitation technique. Clin Chem 1976; 22:98-101, which is incorporated by reference herein.

Plasma plant sterols (sitosterol and campesterol) and LDL-C were assessed at baseline (Day 1) and at endpoint (average of Weeks 6 and 8 values). See: Salen, Gerald; Shore, Virgie; Tint, GS; Forte, T: Shefer, S; Horak, I; Horak, E; Dayal, B; Nguyen, L.; Batta, A K; Lindgren, FT; Kwiterovich, Jr, PO, “Increased sitosterol absorption, decreased removal and expanded body pools compensate for reduced cholesterol synthesis in sitosterolemia with xanthomatosis”, J Lipid Res, Vol. 30, pp 1319-30, (1989) and Lutjohann, D; Bjorkhem, I; Beil, UF, and von Bergmann, K, “Sterol absorption and sterol balance in phytosterolernia evaluated by deuterium-labeled sterols: effect of sitostanol treatment” J Lipid Res. Vol. 36:(8), pp 1763-73, (1995), each of which is incorporated by reference herein.

Results:

The mean (S.E.) percent (%) change from Baseline at endpoint in plant sterols and LDL-C (mean, 95% Cl) are shown in Table 1 below:

TABLE 1

| Treatment | Sitosterol | Campesterol | LDL-C |
|---|---|---|---|
| A | −21.0% (2.8%) | −24.3% (2.9%) | −13.6% (−21.7%, −5.5%) |
| B | 4.0% (5.3%) | 3.2% (5.5%) | 16.7% (31.6%, 64.9%) |
| (control) | | | |

The coadministration of 10 mg of Compound VIII was well tolerated and caused a significant (p<0.001) reduction in sitosterol and campesterol compared to placebo.

Preparation of Compound (VIII)

Step 1): To a solution of (S)-4-phenyl-2-oxazolidinone (41 g, 0.25 mol) in CH₂Cl₂ (200 ml), was added 4-dimethylaminopyridine (2.5 g, 0.02 mol) and triethylamine (84.7 ml, 0.61 mol) and the reaction mixture was cooled to 0° C. Methyl-4-(chloroformyl)butyrate (50 g, 0.3 mol) was added as a solution in CH₂Cl₂ (375 ml) dropwise over 1 h, and the reaction was allowed to warm to 22° C. After 17 h, water and H₂SO₄ (2N, 100 ml), was added the layers were separated, and the organic layer was washed sequentially with NaOH (10%), NaCl (sat'd) and water. The organic layer was dried over MgSO₄ and concentrated to obtain a semicrystalline product.

Step 2): To a solution of TiCl₄ (18.2 ml, 0.165 mol) in CH₂Cl₂ (600 ml) at 0° C., was added titanium isopropoxide (16.5 ml, 0.055 mol). After 15 min, the product of Step 1 (49.0 g, 0.17 mol) was added as a solution in CH₂Cl₂ (100 ml). After 5 min., diisopropylethylamine (DIPEA) (65.2 ml, 0.37 mol) was added and the reaction mixture was stirred at 0° C. for 1 h, the reaction mixture was cooled to −20° C., and 4-benzyloxybenzylidine(4-fluoro)aniline (114.3 g, 0.37 mol) was added as a solid. The reaction mixture was stirred vigorously for 4 h at −20° C., then acetic acid was added as a solution in CH₂Cl₂ dropwise over 15 min, the reaction mixture was allowed to warm to 0° C., and H₂SO₄ (2N) was added. The reaction mixture was stirred an additional 1 h, the layers were separated, washed with water, separated and the organic layer was dried. The crude product was crystallized from ethanol/water to obtain the pure intermediate.

Step 3): To a solution of the product of Step 2 (8.9 g, 14.9 mmol) in toluene (100 ml) at 50° C., was added N,O-bis(trimethylsilyl)acetamide (BSA) (7.50 ml, 30.3 mmol). After 0.5 h, solid TBAF (0.39 g, 1.5 mmol) was added and the reaction mixture stirred at 50° C. for an additional 3 h. The reaction mixture was cooled to 22° C., CH₃OH (10 ml), was added. The reaction mixture was washed with HCl (1N), NaHCO₃ (1N) and NaCl (sat'd.), and the organic layer was dried over MgSO₄.

Step 4): To a solution of the product of Step 3 (0.94 g, 2.2 mmol) in CH₃OH (3 ml), was added water (1 ml) and LiOH.H₂O (102 mg, 2.4 mmole). The reaction mixture was stirred at 22° C. for 1 h and then additional LiOH.H₂O (54 mg, 1.3 mmole) was added. After a total of 2 h, HCl (1N) and EtOAc was added, the layers were separated, the organic layer was dried and concentrated in vacuo. To a solution of the resultant product (0.91 g, 2.2 mmol) in CH₂Cl₂ at 22° C., was added ClCOCOCl (0.29 ml, 3.3 mmol) and the mixture stirred for 16 h. The solvent was removed in vacuo.

Step 5): To an efficiently stirred suspension of 4-fluorophenylzinc chloride (4.4 mmol) prepared from 4-fluorophenylmagnesium bromide (1M in THF, 4.4 ml, 4.4 mmol) and ZnCl₂ (0.6 g, 4.4 mmol) at 4° C., was added tetrakis(triphenyl-phosphine)palladium (0.25 g, 0.21 mmol) followed by the product of Step 4 (0.94 g, 2.2 mmol) as a solution in THF (2 ml). The reaction was stirred for 1 h at 0° C. and then for 0.5 h at 22° C. HCl (1N, 5 ml) was added and the mixture was extracted with EtOAc. The organic layer was concentrated to an oil and purified by silica gel chromatography to obtain 1-(4-fluorophenyl)-4(S)-(4-hydroxyphenyl)-3(R)-(3-oxo-3-phenylpropyl)-2-azetidinone:

HRMS calc'd for C₂₄H₁₉F₂NO₃=408.1429, found 408.1411.

Step 6): To the product of Step 5 (0.95 g, 1.91 mmol) in THF (3 ml), was added (R)-tetrahydro-1-methyl-3,3-diphenyl-1H,3H-pyrrolo-[1,2-c][1,3,2]oxazaborole (120 mg, 0.43 mmol) and the mixture was cooled to −20° C. After 5 min, borohydride-dimethylsulfide complex (2M in THF, 0.85 ml, 1.7 mmol) was added dropwise over 0.5 h. After a total of 1.5 h, CH₃OH was added followed by HCl (1N) and the reaction mixture was extracted with EtOAc to obtain 1-(4-fluorophenyl)-3(R)-[3(S)-(4-fluorophenyl)-3-hydroxypropyl)]-4(S)-[4-(phenylmethoxy)phenyl]-2-azetidinone (compound 6A-1) as an oil. ¹H in CDCl₃ d H3=4.68. J=2.3 Hz. Cl (M⁺H) 500.

Use of (S)-tetra-hydro-1-methyl-3,3-diphenyl-1H,3H-pyrrolo-[1,2-c][1,3,2]oxazaborole gives the corresponding 3(R)-hydroxypropyl azetidinone (compound 6B-1). ¹H in CDCl₃ d H3=4.69. J=2.3 Hz. Cl (M⁺H) 500.

To a solution of compound 6A-1 (0.4 g, 0.8 mmol) in ethanol (2 ml), was added 10% Pd/C (0.03 g) and the reaction mixture was stirred under a pressure (60 psi) of H₂ gas for 16 h. The reaction mixture was filtered and the solvent was concentrated to obtain compound 6A. Mp 164-166° C.; Cl (M⁺H) 410. [α]_{D}²⁵=−28.1° (c 3, CH₃OH). Elemental analysis calc'd for C₂₄H₂₁F₂NO₃: C, 70.41; H, 5.17; N, 3.42; found C, 70.25; H, 5.19; N, 3.54.

Similarly treat compound 6B-1 to obtain compound 6B.

Mp 129.5-132.5° C.; Cl (M⁺H) 410. Elemental analysis calc'd for C₂₄H₂₁F₂NO₃: C, 70.41; H, 5.17; N, 3.42; found C, 70.30; H, 5.14; N, 3.52.

Step 6′ (Alternative): To a solution of the product of Step 5 (0.14 g, 0.3 mmol) in ethanol (2 ml), was added 10% Pd/C (0.03 g) and the reaction was stirred under a pressure (60 psi) of H₂ gas for 16 h. The reaction mixture was filtered and the solvent was concentrated to afford a 1:1 mixture of compounds 6A and 6B.

It will be appreciated by those skilled in the art that changes could be made to the embodiments described above without departing from the broad inventive concept thereof. It is understood, therefore, that this invention is not limited to the particular embodiments disclosed, but it is intended to cover modifications which are within the spirit and scope of the invention, as defined by the appended claims.

We claim:

1. A pharmaceutical composition comprising an effective amount of at least one sterol absorption inhibiting compound or a prodrug or a pharmaceutically acceptable salt thereof and an effective amount of at least one CETP inhibitor of the formula (I′): 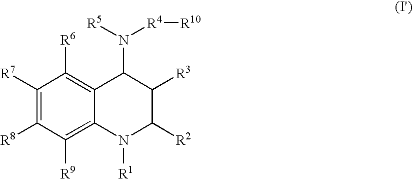
wherein R¹ is a hydrogen atom, an optionally substituted alkoxycarbonyl group, an optionally substituted carbamoyl group, an optionally substituted alkyl group, an optionally substituted alkanoyl group, a saturated or unsaturated monocyclic or bicyclic heterocyclic group containing 1 to 4 heteroatoms selected independently from oxygen, sulfur and nitrogen atoms (the heterocyclic group is optionally substituted), or a saturated or unsaturated monocyclic or bicylci heterocyclic carbonyl group containing 1 to 4 heteroatoms selected independently from oxygen, sulfur and nitrogen atoms (the heterocyclic group is optionally substituted);
R² is a hydrogen atom or an optionally substituted alkyl group;
R³ is a hydrogen atom or an optionally substituted alkyl group;
R⁴ is an optionally substituted alkylene group;
R⁵ is a saturated or unsaturated monocyclic or bicyclic heterocyclic group containing 1 to 4 heteroatoms selected independently from oxygen, sulfur and nitrogen atoms, wherein the heterocyclic group is substituted by 1 to 5 substituents selected from the following groups, or said heterocyclic group is substituted by 1 to 5 substituents selected from the following groups and further by a halogen atom, an oxo and/or hydroxyl group;
cyano group, nitro group, carboxyl group, sulfo group, C₃₋₁₀ alkyl group, substituted alkyl group, optionally substituted cycloalkyl group, optionally substituted alkenyl group, C₃₋₁₀ alkoxy group, substituted alkoxy group, optionally substituted cycloalkoxy group, optionally substituted alkoxycarbonyl group, optionally substituted carbamoyl group, optionally substituted carbamimidoyl group, optionally substituted alkylthio group, optionally substituted alkylsulfinyl group, optionally substituted alkylsulfonyl group, optionally substituted alikylsuflonyl group, optionally substituted amino group, optionally substituted sulfamoyl group, optionally substituted alkanoyl group, a saturated or unsaturated monocyclic or bicyclic heterocyclic group containing 1 to 4 heteroatoms selected independently from oxygen, sulfur and nitrogen atoms (the heterocyclic group is optionally substituted), a saturated or unsaturated monocyclic or bicyclic heterocyclic oxy group containing 1 to 4 heteroatoms selected independently from oxygen, sulfur and nitrogen atoms (the heterocyclic oxy group is optionally substituted), and a saturated or unsaturated monocyclic or bicyclic heterocyclic carbonyl group containing 1 to 4 heterocyclic carbonyl group is optionally substituted);
R⁶ and R⁷, or R⁷ and R₈, or R⁸ and R⁹ may combine at the ends to form an alkylene group which alkylene group may contain 1 to 3 heteroatoms selected independently from nitrogen, sulfur and oxygen atoms, and may have a substituent(s); and
R₁₀ is an aromatic ring optionally containing 1 to 3 heteroatoms selected independently from oxygen, sulfur and nitrogen atoms (the aromatic ring is optionally substituted),
or a pharmaceutically acceptable salt thereof.

2. The pharmaceutical composition of claim 1, wherein the sterol absorption inhibitor is represented by Formula (I) 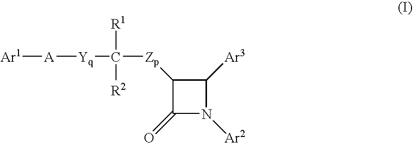
or isomers thereof, or pharmaceutically acceptable salts or solvates of the compounds of Formula (I) or of the isomers thereof, or prodrugs of the compounds of Formula (I) or of the isomers, salts or solvates thereof, wherein:
Ar¹ is R³-substituted aryl;
Ar² is R⁴-substituted aryl;
Ar³ is R⁵-substituted aryl;
Y and Z are independently selected from the group consisting of —CH₂—, —CH(lower alkyl)- and —C(dilower alkyl)-;
A is —O—, —S—, —S(O)— or —S(O)₂—;
R¹ is selected from the group consisting of —OR⁶, —O(CO)R⁶, —O(CO)OR⁹ and —O(CO)NR⁶R⁷;
R² is selected from the group consisting of hydrogen, lower alkyl and aryl; or
R¹ and R² together are ═O;
q is 1, 2 or 3;
p is 0, 1, 2, 3 or 4;
R⁵ is 1-3 substituents independently selected from the group consisting of —OR⁶, —O(CO)R⁶, —O(CO)OR⁹, —O(CH₂)₁₋₅OR⁹, —O(CO)NR⁶R⁷, —NR⁶R⁷, —NR⁶(CO)R⁷, —NR⁶(CO)OR⁹, —NR⁶(CO)NR⁷R⁸, —NR⁶SO₂-lower alkyl, —NR⁶SO₂-aryl, —CONR⁶R⁷, —COR⁶, —SO₂NR⁶R⁷, S(O)₀₋₂-alkyl, S(O)₀₋₂-aryl, —O(CH₂)₁₋₁₀—COOR⁶, —O(CH₂)₁₋₁₀CONR⁶R⁷, o-halogeno, m-halogeno, o-lower alkyl, m-lower alkyl, -(lower alkylene)-COOR⁶, and —CH═CH—COOR⁶;
R³ and R⁴ are independently 1-3 substituents independently selected from the group consisting of R⁵, hydrogen, p-lower alkyl, aryl, —NO₂, —CF₃ and p-halogeno;
R⁶, R⁷ and R⁸ are independently selected from the group consisting of hydrogen, lower alkyl, aryl and aryl-substituted lower alkyl; and
R⁹ is lower alkyl, aryl or aryl-substituted lower alkyl.

3. The pharmaceutical composition of claim 1, wherein the sterol absorption inhibitor is a compound of the formula represented by Formula (VIII)

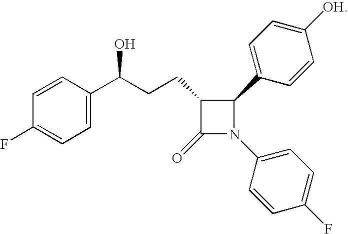

4. The pharmaceutical composition of claim 1, wherein the sterol absorption inhibitor is represented by Formula (II) 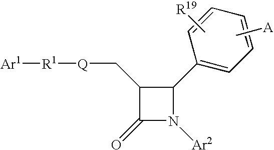
or isomers thereof, or pharmaceutically acceptable salts or solvates of the compounds of Formula (II) or of the isomers thereof, or prodrugs of the compounds of Formula (II) or of the isomers, salts or solvates thereof, wherein:
A is selected from the group consisting of R²-substituted heterocycloalkyl, R²-substituted heteroaryl, R²-substituted benzofused heterocycloalkyl, and R²-substituted benzofused heteroaryl;
Ar¹ is aryl or R³-substituted aryl;
Ar² is aryl or R⁴-substituted aryl;
Q is a bond or, with the 3-position ring carbon of the azetidinone, forms the spiro group 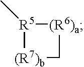
R₁ is selected from the group consisting of
—(CH₂)_{q}—, wherein q is 2-6, provided that when Q forms a spiro ring, q can also be zero or 1;
—(CH₂)ₑ-G-(CH₂)ᵣ—, wherein G is —O—, —C(O)—, phenylene, —NR⁸— or —S(O)₀₋₂-e is 0-5 and r is 0-5, provided that the sum of e and r is 1-6;
—(C₂-C₆ alkenylene)-; and
—(CH₂)_{f}—V—(CH₂)_{g}—, wherein V is C₃-C₆ cycloalkylene, f is 1-5 and g is 0-5, provided that the sum of f and g is 1-6;
R⁵ is 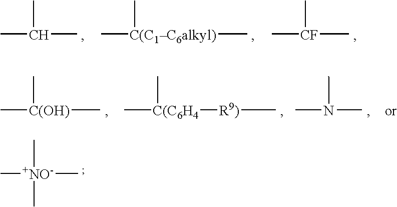
R⁶ and R⁷ are independently selected from the group consisting of —CH₂—, —CH(C₁-C₆ alkyl)-, —C(di-(C₁-C₆) alkyl), —CH═CH— and —C(C₁-C₆ alkyl)=CH—; or R₅ together with an adjacent R₆, or R₅ together with an adjacent R⁷, form a —CH═CH— or a —CH═C(C₁-C₆ alkyl)- group;
a and b are independently 0, 1, 2 or 3, provided both are not zero; provided that when R⁶ is —CH═CH— or —C(C₁-C₆ alkyl)=CH—, a is 1; provided that when R⁷ is —CH═CH— or —C(C₁-C₆ alkyl)=CH—, b is 1; provided that when a is 2 or 3, the R⁶'s can be the same or different; and provided that when b is 2 or 3, the R⁷'s can be the same or different;
and when Q is a bond, R₁ also can be: 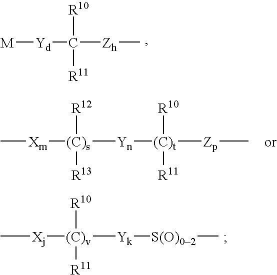
M is —O—, —S—, —S(O)— or —S(O)₂—;
X, Y and Z are independently selected from the group consisting of —CH₂—, —CH(C₁-C₆ alkyl)- and —C(di-(C₁-C₆) alkyl);
R¹⁰ and R¹² are independently selected from the group consisting of —OR¹⁴, —O(CO)R¹⁴, —O(CO)OR¹⁶ and —O(CO)NR¹⁴R¹⁵;
R¹¹ and R¹³ are independently selected from the group consisting of hydrogen, (C₁-C₆)alkyl and aryl; or R¹⁰ and R¹¹ together are ═O, or
R¹² and R¹³ together are ═O;
d is 1, 2 or 3;
h is 0, 1, 2, 3 or 4;
s is 0 or 1; t is 0 or 1; m, n and p are independently 0-4; provided that at least one of s and t is 1, and the sum of m, n, p, s and t is 1-6; provided that when p is 0 and t is 1, the sum of m, s and n is 1-5; and provided that when p is 0 and s is 1, the sum of m, t and n is 1-5;
v is 0 or 1;
j and k are independently 1-5, provided that the sum of j, k and v is 1-5;
R² is 1-3 substituents on the ring carbon atoms selected from the group consisting of hydrogen, (C₁-C₁₀)alkyl, (C₂-C₁₀)alkenyl, (C₂-C₁₀)alkynyl, (C₃-C₆)cycloalkyl, (C₃-C₆)cycloalkenyl, R¹⁷-substituted aryl, R¹⁷-substituted benzyl, R¹⁷-substituted benzyloxy, R¹⁷-substituted aryloxy, halogeno, —NR¹⁴R¹⁵, NR¹⁴R¹⁵(C₁-C₆ alkylene)-, NR¹⁴R¹⁵C(O)(C₁-C₆ alkylene)-, —NHC(O)R¹⁶, OH, C₁-C₆ alkoxy, —OC(O)R¹⁶, —COR¹⁴, hydroxy(C₁-C₆)alkyl, (C₁-C₆)alkoxy(C₁-C₆)alkyl, NO₂, —S(O)₀₋₂R¹⁶, —SO₂NR¹⁴R¹⁵ and —(C₁-C₆ alkylene)COOR¹⁴; when R² is a substituent on a heterocycloalkyl ring, R² is as defined, or is ═O or 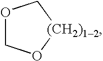
and, where R² is a substituent on a substitutable ring nitrogen, it is hydrogen, (C₁-C₆)alkyl, aryl, (C₁-C₆)alkoxy, aryloxy, (C₁-C₆)alkylcarbonyl, arylcarbonyl, hydroxy, —(CH₂)₁₋₆CONR¹⁸R¹⁸, 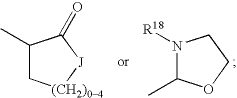
wherein J is —O—, —NH—, —NR¹⁸— or —CH₂—;
R³ and R⁴ are independently selected from the group consisting of 1-3 substituents independently selected from the group consisting of (C₁-C₆)alkyl, —OR¹⁴, —O(CO)R¹⁴, —O(CO)OR¹⁶, —O(CH₂)₁₋₅OR¹⁴, —O(CO)NR¹⁴R¹⁵, —NR¹⁴R¹⁵, ——NR¹⁴(CO)R¹⁵, —NR¹⁴(CO)OR¹⁶, —NR¹⁴(CO)NR¹⁵R¹⁹, —NR¹⁴SO₂R¹⁶, —COOR¹⁴, CONR¹⁴R¹⁵, —COR¹⁴, —SO₂NR¹⁴R¹⁵, S(O)₀₋₂R¹⁶, —O(CH₂)₁₋₁₀—COOR¹⁴, —O(CH₂)₁₋₁₀CONR¹⁴R¹⁵, —(C₁-C₆ alkylene)-COOR¹⁴, —CH═CH—COOR¹⁴, —CF₃, —CN, —NO₂ and halogen;
R⁸ is hydrogen, (C₁-C₆)alkyl, aryl (C₁-C₆)alkyl, —C(O)R¹⁴ or —COOR¹⁴;
R⁹ and R¹⁷ are independently 1-3 groups independently selected from the group consisting of hydrogen, (C₁-C₆)alkyl, (C₁-C₆)alkoxy, —COOH, NO₂, —NR¹⁴R¹⁵, OH and halogeno;
R¹⁴ and R¹⁵ are independently selected from the group consisting of hydrogen, (C₁-C₆)alkyl, aryl and aryl-substituted (C₁-C₆)alkyl;
R¹⁶ is (C₁-C₆)alkyl, aryl or R¹⁷-substituted aryl;
R¹⁸ is hydrogen or (C₁-C₆)alkyl; and
R¹⁹ is hydrogen, hydroxy or (C₁-C₆)alkoxy.

5. The pharmaceutical composition of claim 1, wherein the sterol absorption inhibitor is represented by Formula (III) 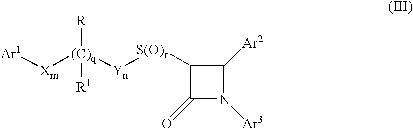
or isomers thereof, or pharmaceutically acceptable salts or solvates of the compounds of Formula (III) or of the isomers thereof, or prodrugs of the compounds of Formula (III) or of the isomers, salts or solvates thereof, wherein:
Ar¹ is aryl, R¹⁰-substituted aryl or heteroaryl;
Ar² is aryl or R⁴-substituted aryl;
Ar³ is aryl or R⁵-substituted aryl;
X and Y are independently selected from the group consisting of —CH₂—, —CH(lower alkyl)- and —C(dilower alkyl)-;
R is —OR⁶, —O(CO)R⁶, —O(CO)OR⁹ or —O(CO)NR⁶R⁷;
R¹ is hydrogen, lower alkyl or aryl; or R and R¹ together are ═O;
q is 0 or 1;
r is 0, 1 or 2;
m and n are independently 0, 1, 2, 3, 4 or 5; provided that the sum of m, n and q is 1, 2, 3, 4 or 5;
R⁴ is 1-5 substituents independently selected from the group consisting of lower alkyl, —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, -(lower alkylene)COOR⁶ and —CH═CH—COOR⁶;
R⁵ is 1-5 substituents independently selected from the group consisting of —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, —CF₃, —CN, —NO₂, halogen, -(lower alkylene)COOR⁶ and —CH═CH—COOR⁶;
R⁶, R⁷ and R⁸ are independently selected from the group consisting of hydrogen, lower alkyl, aryl and aryl-substituted lower alkyl;
R⁹ is lower alkyl, aryl or aryl-substituted lower alkyl; and
R¹⁰ is 1-5 substituents independently selected from the group consisting of lower alkyl, —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, —CF₃, —CN, —NO₂ and halogen.

6. The pharmaceutical composition of claim 1, wherein the sterol absorption inhibitor is represented by Formula (IVA) 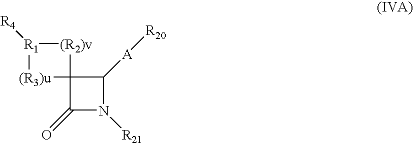
or isomers thereof, or pharmaceutically acceptable salts or solvates of the compounds of Formula (IVA) or of the isomers thereof, or prodrugs of the compounds of Formula (IVA) or of the isomers, salts or solvates thereof, wherein:
R₁ is 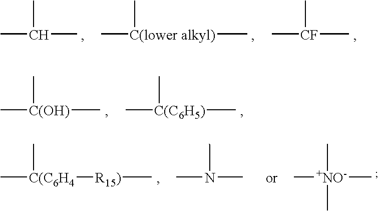
R₂ and R₃ are independently selected from the group consisting of —CH₂—, —CH(lower alkyl)-, —C(di-lower alkyl)-, —CH═CH— and —C(lower alkyl)=CH—; or
R₁ and R₂ together or R₁ and R₃ together form a —CH═CH— or a —CH═C(lower alkyl)- group;
u and v are independently 0, 1, 2 or 3, provided both are not zero;
R₄ is
B—(CH₂)ₘC(O)—, wherein m is 0, 1, 2, 3, 4 or 5;
B—(CH₂)_{q}—, wherein q is 0, 1, 2, 3, 4, 5 or 6;
B—(CH₂)e-Z-(CH₂)ᵣ—, wherein Z is —O—, —C(O)—, phenylene,
—NR₈— or —S(O)₀₋₂—, and wherein e is 0, 1, 2, 3, 4 or 5 and r is 0, 1, 2, 3, 4 or 5, provided that the sum of e and r is 0, 1, 2, 3, 4, 5 or 6;
B—(C₂-C₆ alkenylene)-;
B′—(C₄-C₆ alkadienylene)-;
B—(CH₂)ₜ-Z-(C₂-C₆ alkenylene)-, wherein Z is as defined above, and wherein t is 0, 1, 2 or 3, provided that the sum of t and the number of carbon atoms in the alkenylene chain is 2, 3, 4, 5 or 6;
B—(CH₂)_{f}—V—(CH₂)_{g}—, wherein V is C₃-C₆ cycloalkylene, f is 1, 2, 3, 4 or 5 and g is 0, 1, 2, 3, 4 or 5, provided that the sum of f and g is 1, 2, 3, 4, 5 or 6;
B—(CH₂)ₜ—V—(C₂-C₆ alkenylene)- or B′—(C₂-C₆ alkenylene)-V-(CH₂)ₜ—, wherein V and t are as defined above, provided that the sum of t and the number of carbon atoms in the alkenylene chain is 2, 3, 4, 5 or 6;
B—(CH₂)ₐ-Z-(CH₂)_{b}—V—(CH₂)_{d}—, wherein Z and V are as defined above and a, b and d are independently 0, 1, 2, 3, 4, 5 or 6, provided that the sum of a, band d is 0, 1, 2, 3, 4, 5 or 6;
T-(CH₂)ₛ—, wherein T is cycloalkyl of 3-6 carbon atoms and s is 1, 2, 3, 4, 5 or 6; or naphthylmethyl, heteroarylmethyl, or W-substituted heteroarylmethyl, wherein heteroaryl is selected from the group consisting of pyrrolyi, pyridinyl, pyrimidinyl, pyrazinyl, triazinyl, imidazolyl, thiazolyl, pyrazolyl, thienyl, oxazolyl and furanyl, and for nitrogen-containing heteroaryls, the N-oxides thereof;
and wherein W is 1 to 3 substituents independently selected from the group consisting of lower alkyl, hydroxy lower alkyl, lower alkoxy, alkoxyalkyl, alkoxyalkoxy, alkoxycarbonylalkoxy, (lower alkoxyimino)-lower alkyl, lower alkanedioyl, lower alkyl lower alkanedioyl, allyloxy, —CF₃, —OCF₃, benzyl, R₇-benzyl, benzyloxy, R₇-benzyloxy, phenoxy, R₇-phenoxy, dioxolanyl, NO₂, —NR₈R₉, NR₈R₉(lower alkyl)-, NR₈R₉(lower alkoxy)-, OH, halogeno, —NHC(O)OR₁₀, —NHC(O)R₁₀, R₁₁O₂SNH—, (R₁₁O₂S)₂N—,
—S(O)₂NH₂, —S(O)₀₋₂R₈, tert-butyldimethyl-silyloxymethyl, —C(O)R₁₂, —CH═CHC(O)R₁₂, R₁₀C(O)(lower alkoxy)-, NR₈R₉C(O)(lower alkoxy)- and 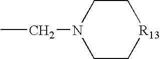
for substitution on ring carbon atoms, and the substituents on the substituted heteroaryl ring nitrogen atoms, when present, are selected from the group consisting of lower alkyl, lower alkoxy, —C(O)OR₁₀, —C(O)R₁₀, OH, NR₈R₉(lower alkyl)-, NR₈R₉(lower alkoxy)-, —S(O)₂NH₂ and 2-(trimethylsilyl)ethoxymethyl;
A is a bond; C₃-C₆ cycloalkylene; C₁-C₁₀ alkylene; C₂-C₁₀ alkenylene; C₂-C₁₀ alkynylene; an alkylene, alkenylene or alkynylene chain as defined above, substituted by 1 to 4 substituents independently selected from the group consisting of
phenyl,
W-substituted phenyl,
heteroaryl and
W-substituted heteroaryl,
wherein heteroaryl is as defined above; an alkylene, alkenylene or alkynylene chain as defined above interrupted by 1 to 4 groups independently selected from the group consisting of
—O—,
—S—,
—SO—,
—SO₂—,
—NR₁₄,
—C(O)—,
C₃-C₆ cycloalkylene,
phenylene,
W-substituted phenylene,
heteroarylene and
W-substituted heteroarylene; or
an interrupted alkylene,
alkenylene or
alkynylene chain as defined substituted by 1 to 4 substituents independently selected from the group consisting of
phenyl,
W-substituted phenyl,
heteroaryl and
W-substituted heteroaryl;
B is naphthyl, heteroaryl or W-substituted heteroaryl, wherein heteroaryl is as defined above, or 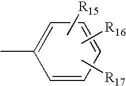
R₇ is 1-3 groups independently selected from the group consisting of lower alkyl, lower alkoxy, —COOH, NO₂, —NR₈R₉, OH or halogeno;
R₈ and R₉ are independently H or lower alkyl;
R₁₀ is lower alkyl, phenyl, R₇-phenyl, benzyl or R₇-benzyl;
R¹¹ is OH, lower alkyl, phenyl, benzyl, R₇-phenyl or R₇-benzyl;
R₁₂ is H, OH, alkoxy, phenoxy, benzyloxy, 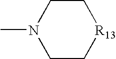
NR₈R₉, lower alkyl, phenyl or R₇-phenyl;
R₁₃ is —O—, —CH₂—, —NH—, —N(lower alkyl)- or —NC(O)R₁₉;
R₁₄ is H, lower alkyl, phenyl lower alkyl or —C(O)R₁₉;
R₁₅, R₁₆ and R₁₇ are independently selected from the group consisting of H and the groups defined for W; or R₁₅ is hydrogen and R₁₆ and R₁₇, together with adjacent carbon atoms to which they are attached, form a dioxolanyl ring;
R₁₉ is H, lower alkyl, phenyl or phenyl lower alkyl;
R₂₁ is
phenyl,
W-substituted phenyl,
naphthyl,
W-substituted naphthyl,
benzodioxolyl,
heteroaryl,
W-substituted heteroaryl,
benzofused heteroaryl,
W-substituted benzofused heteroaryl or
cyclopropyl,
wherein heteroaryl is as defined above; and
R₂₀ is H or R₂₁.

7. The pharmaceutical composition of claim 1, wherein the sterol absorption inhibitor is represented by Formula (IVB) 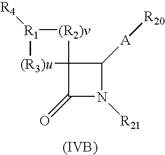
or isomers thereof, or pharmaceutically acceptable salts or solvates of the compounds of Formula (IVB) or of the isomers thereof, or prodrugs of the compounds of Formula (IVB) or of the isomers, salts or solvates thereof, wherein:
R₁, R₂, R₃, R₄, u and v are as defined for Formula (IVA),
R₅ is —CH═CH—B′, wherein B′ is 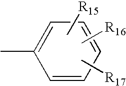
and R₁₅, R₁₆ and R₁₇ are as defined for Formula (IVA);
—C≡C—B′;
—(CH₂)ₚ—X—B′, wherein p is 0, 1 or 2 and X is a bond, —NH— or
—S(O)_{0-2—;}
—C(O)—B′;
heteroaryl,
benzofused heteroaryl,
W-substituted heteroaryl or
W-substituted benzofused heteroaryl,
wherein heteroaryl and W are as defined for Formula (IVA); or 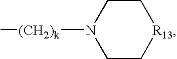
wherein k is 1 or 2 and R₁₃ is as defined for Formula (IVA); and
R₆ is 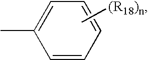
indanyl,
benzofuranyl,
benzodioxolyl,
tetrahydronaphthyl,
pyridyl,
pyrazinyl,
pyrimidinyl,
quinolyl or
cyclopropyl;
n is 0, 1, 2 or 3;
R₁₈ is
lower alkyl,
lower alkoxy,
OH,
halogeno,
—NR₈R₉,
—NHC(O)OR₁₀,
—NHC(O)R₁₀,
NO₂,
—CN,
—N₃,
—SH,
—S(O)₀₋₂-(lower alkyl),
—COOR₁₉,
—CONR₈R₉,
—COR₁₂,
phenoxy,
benzyloxy,
—CH═CHC(O)R₁₂,
—OCF₃ or
tert-butyl-dimethyl-silyloxy,
wherein when n is 2 or 3, the R₁₈ groups can be the same or different, and wherein R₈, R₉, R₁₀, R₁₂ and R₁₉ are as defined in Formula (IVA).

8. The pharmaceutical composition of claim 1, wherein the sterol absorption inhibitor is represented by Formula (VA) or Formula (VB) 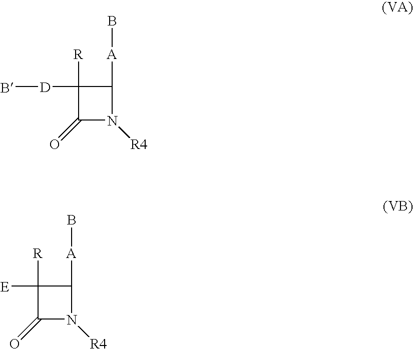
or isomers thereof, or pharmaceutically acceptable salts or solvates of the compounds of Formula (VA) and (VB) or of the isomers thereof, or prodrugs of the compounds of Formula (VA) and (VB) or of the isomers, salts or solvates thereof, wherein:
A is —CH═CH—, —C≡C— or —(CH₂)ₚ— wherein p is 0, 1 or 2;
B is 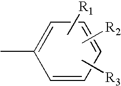
B′ is 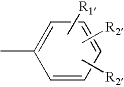
D is —(CH₂)ₘC(O)— or —(CH₂)_{q}— wherein m is 1, 2, 3 or 4 and q is 2, 3 or 4;
E is C₁₀ to C₂₋₀ alkyl or —C(O)-(C₉ to C₁₉)-alkyl, wherein the alkyl is straight or branched, saturated or containing one or more double bonds;
R is hydrogen, C₁-C₁₅ alkyl, straight or branched, saturated or containing one or more double bonds, or B—(CH₂)ᵣ—, wherein r is 0, 1, 2, or 3;
R₁, R₂, R₃, R_{1′}, R_{2′}, and R_{3′} are independently selected from the group consisting of
hydrogen,
lower alkyl,
lower alkoxy,
carboxy,
NO₂,
NH₂,
OH,
halogeno,
lower alkylamino,
dilower alkylamino,
—NHC(O)OR₅,
R₆O₂SNH— and
—S(O)₂NH₂;
R₄ is 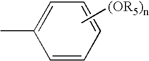
wherein n is 0, 1, 2 or 3;
R₅ is lower alkyl; and
R₆ is OH,
lower alkyl,
phenyl,
benzyl or
substituted phenyl
wherein the substituents are 1-3 groups independently selected from the group
consisting of
lower alkyl,
lower alkoxy,
carboxy,
NO₂,
NH₂,
OH,
halogeno,
lower alkylamino and
dilower alkylamino.

9. The pharmaceutical composition of claim 1, wherein the sterol absorption inhibitor is represented by Formula (VI) 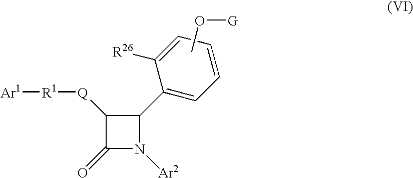
or isomers thereof, or pharmaceutically acceptable salts or solvates of the compounds of Formula (VI) or of the isomers thereof, or prodrugs of the compounds of Formula (VI) or of the isomers, salts or solvates thereof, wherein:
R²⁶ is H or OG¹;
G and G¹ are independently selected from the group consisting of
H, 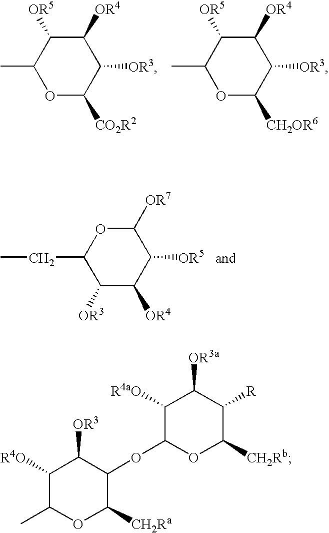
provided that when R²⁶ is H or OH, G is not H;
R, R^{a} and R^{b} are independently selected from the group consisting of
H,
—OH,
halogeno,
—NH₂,
azido,
(C₁-C₆)alkoxy(C₁-C₆)-alkoxy and
—W—R³⁰;
wherein W is independently selected from the group consisting of
—NH—C(O)—,
—O—C(O)—,
—O—C(O)—N(R³¹)—,
—NH—C(O)—N(R³¹)— and
—O—C(S)—N(R³¹)—;
R² and R⁶ are independently selected from the group consisting of
H,
(C₁-C₆)alkyl,
aryl and
aryl(C₁-C₆)alkyl;
R³, R⁴, R⁵, R⁷, R₃ₐ and R^{4a} are independently selected from the group consisting of H,
(C₁-C₆)alkyl,
aryl(C₁-C₆)alkyl,
—C(O)(C₁-C₆)alkyl and
—C(O)aryl;
R³⁰ is selected from the group consisting of
R³²-substituted T,
R³²-substituted-T-(C₁-C₆)alkyl,
R³²-substituted-(C₂-C₄)alkenyl,
R³²-substituted-(C₁-C₆)alkyl,
R³²-substituted-(C₃-C₇)cycloalkyl and
R³²-substituted-(C₃-C₇)cycloalkyl(C₁-C₆)alkyl;
R³¹ is selected from the group consisting of H and (C₁-C₄)alkyl;
T is selected from the group consisting of
phenyl,
furyl,
thienyl,
pyrrolyl,
oxazolyl,
isoxazolyl,
thiazolyl,
iosthiazolyl,
benzothiazolyl,
thiadiazolyl,
pyrazolyl,
imidazolyl and
pyridyl;
R³² is independently selected from 1-3 substituents independently selected from the group consisting of
halogeno,
(C₁-C₄)alkyl,
—OH, phenoxy,
—CF₃,
—NO₂,
(C₁-C₄)alkoxy,
methylenedioxy,
oxo,
(C₁-C₄)alkylsulfanyl,
(C₁-C₄)alkylsulfinyl,
(C₁-C₄)alkylsulfonyl,
—N(CH₃)₂,
—C(O)—NH(C₁-C₄)alkyl,
—C(O)—N((C₁-C₄)alkyl)₂,
—C(O)—(C₁-C₄)alkyl,
—C(O)—(C₁-C₄)alkoxy and
pyrrolidinylcarbonyl; or
R³² is a covalent bond and R³¹, the nitrogen to which it is attached and R³² form a
pyrrolidinyl,
piperidinyl,
N-methyl-piperazinyl,
indolinyl or
morpholinyl group,
or a
(C₁-C₄)alkoxycarbonyl-substituted
pyrrolidinyl,
piperidinyl,
N-methylpiperazinyl,
indolinyl or
morpholinyl group;
Ar¹ is aryl or R¹⁰-substituted aryl;
Ar² is aryl or R¹¹-substituted aryl;
Q is a bond or, with the 3-position ring carbon of the azetidinone,
forms the spiro group 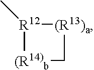
and
R¹ is selected from the group consisting of
—(CH₂)_{q}—, wherein q is 2-6, provided that when Q forms a spiro ring, q can also be zero or 1;
—(CH₂)ₑ-E-(CH₂)ᵣ—, wherein E is —O—, —C(O)—, phenylene, —NR²²— or —S(O)₀₋₂—, e is 0-5 and r is 0-5, provided that the sum of e and r is 1-6;
—(C₂-C₆)alkenylene-; and
—(CH₂)_{f}—V—(CH₂)_{g}—, wherein V is C₃-C₆ cycloalkylene, f is 1-5 and g is 0-5, provided that the sum of f and g is 1-6;
R¹² is 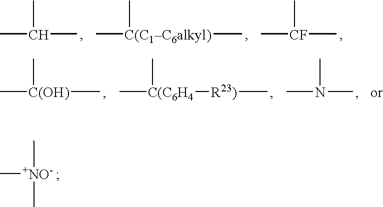
R¹³ and R¹⁴ are independently selected from the group consisting of
—CH₂—,
—CH(C₁-C₆ alkyl)-,
—C(di-(C₁-C₆) alkyl),
—CH═CH— and
—C(C₁-C₆ alkyl)=CH—; or
R¹² together with an adjacent R¹³, or R¹² together with an adjacent R¹⁴, form a —CH═CH— or a —CH═C(C₁-C₆ alkyl)- group;
a and b are independently 0, 1, 2 or 3, provided both are not zero;
provided that when R¹³ is —CH═CH— or —C(C₁-C₆ alkyl)=CH—, a is 1;
provided that when R¹⁴ is —CH═CH— or —C(C₁-C₆ alkyl)=CH—, b is 1;
provided that when a is 2 or 3, the R¹³'s can be the same or different; and
provided that when b is 2 or 3, the R¹⁴′s can be the same or different;
and when 0 is a bond. R¹ also can be: 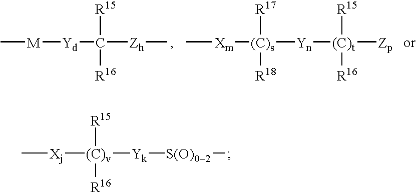
M is —O—, —S—, —S(O)— or —S(O)₂—;
X, Y and Z are independently selected from the group consisting of —CH₂—, —CH(C₁-C₆)alkyl- and —C(di-(C₁-C₆)alkyl);
R₁₀ and R¹¹ are independently selected from the group consisting of 1-3 substituents independently selected from the group consisting of
(C₁-C₆)alkyl,
—OR¹⁹,
—O(CO)R¹ ⁹,
—O(CO)OR²¹,
—O(CH₂)₁₋₅OR¹⁹,
—O(CO)NR¹⁹R²⁰,
—NR¹⁹R²⁰,
—NR¹⁹(CO)R²⁰,
—NR¹⁹(CO)OR²¹,
—NR¹⁹(CO)NR²⁰R²⁵,
—NR¹⁹SO₂R²¹,
—COOR¹⁹,
—CONR¹⁹R²⁰,
—COR¹⁹,
—SO₂NR¹⁹R²⁰,
S(O)₀₋₂R²¹,
—O(CH₂)₁₋₁₀—COOR¹⁹,
—O(CH₂)₁₋₁₀CONR¹⁹R²⁰,
—(C₁-C₆ alkylene)-COOR¹⁹,
—CH═CH—COOR¹⁹,
—CF₃,
—CN,
—NO₂ and
halogen;
R¹⁵ and R¹⁷ are independently selected from the group consisting of —OR¹⁹, —O(CO)R¹⁹, —O(CO)OR²¹ and —O(CO)NR¹⁹R²⁰; R¹⁶ and R¹⁸ are independently selected from the group consisting of H, (C₁-C₆)alkyl and aryl; or R¹⁵ and R¹⁶ together are ═O, or R¹⁷ and R¹⁸ together are ═O;
d is 1, 2 or 3;
h is 0, 1, 2, 3 or 4;
s is 0 or 1; t is 0 or 1; m, n and p are independently 0-4; provided that at least one of s and t is 1, and the sum of m, n, p, s and t is 1-6; provided that when p is 0 and t is 1, the sum of m, s and n is 1-5; and provided that when p is 0 and s is 1, the sum of m, t and n is 1-5;
v is 0 or 1;
j and k are independently 1-5, provided that the sum of j, k and v is 1-5; and when Q is a bond and R¹ is 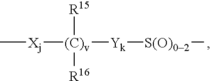
Ar¹ can also be
pyridyl,
isoxazolyl,
furanyl,
pyrrolyl,
thienyl,
imidazolyl,
pyrazolyl,
thiazolyl,
pyrazinyl,
pyrimidinyl or
pyridazinyl;
R¹⁹ and R²⁰ are independently selected from the group consisting of H, (C₁-C₆)alkyl, aryl and aryl-substituted (C₁-C₆)alkyl;
R²¹ is (C₁-C₆)alkyl, aryl or R²⁴-substituted aryl;
R²² is H, (C₁-C₆)alkyl, aryl (C₁-C₆)alkyl, —C(O)R¹⁹ or —COOR¹⁹;
R²³ and R²⁴ are independently 1-3 groups independently selected from the group consisting of H, (C₁-C₆)alkyl, (C₁-C₆)alkoxy, —COOH, NO₂, —NR¹⁹R²⁰, —OH and halogeno; and
R²⁵ is H, —OH or (C₁-C₆)alkoxy.

10. The pharmaceutical composition of claim 1, wherein the sterol absorption inhibitor is represented by Formula (VII) 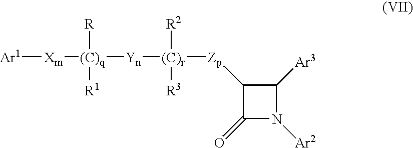
or isomers thereof, or pharmaceutically acceptable salts or solvates of the compounds of Formula (VII) or of the isomers thereof, or prodrugs of the compounds of Formula (VII) or of the isomers, salts or solvates thereof, wherein:
Ar¹ and Ar² are independently selected from the group consisting of aryl and R⁴-substituted aryl;
Ar³ is aryl or R⁵-substituted aryl;
X, Y and Z are independently selected from the group consisting of —CH₂—, —CH(lower alkyl)- and —C(dilower alkyl)-;
R and R² are independently selected from the group consisting of —OR⁶, —O(CO)R⁶, —O(CO)OR⁹ and —O(CO)NR⁶R⁷;
R¹ and R³ are independently selected from the group consisting of hydrogen, lower alkyl and aryl;
q is 0 or 1;
r is 0 or 1;
m, n and p are independently 0, 1, 2, 3 or 4;
provided that at least one of q and r is 1, and the sum of m, n, p, q and r is 1, 2, 3, 4, 5 or 6; and
provided that when p is 0 and r is 1, the sum of m, q and n is 1, 2, 3, 4 or 5;
R⁴ is 1-5 substituents independently selected from the group consisting of
lower alkyl,
—OR⁶,
—O(CO)R⁶,
—O(CO)OR⁹,
—O(CH₂)₁₋₅OR⁶,
—O(CO)NR⁶R⁷,
—NR⁶R⁷,
—NR⁶(CO)R⁷,
—NR⁶(CO)OR⁹,
—NR⁶(CO)NR⁷R⁸,
—NR⁶SO₂R⁹,
—COOR⁶,
—CONR⁶R⁷,
—COR⁶,
—SO₂NR⁶R⁷,
—S(O)₀₋₂R⁹,
—O(CH₂)₁₋₁₀—COOR⁶,
—O(CH₂)₁₋₁₀CONR⁶R⁷,
-(lower alkylene)COOR⁶,
—CH═CH—COOR⁶,
—CF₃,
—CN,
—NO₂ and
halogen;
R⁵ is 1-5 substituents independently selected from the group consisting of
—O(CO)R⁶,
—O(CO)OR⁹,
—O(CH₂)₁₋₅OR⁶,
—O(CO)NR⁶R⁷,
—NR⁶R⁷,
—NR⁶(CO)R⁷,
—NR⁶(CO)OR⁹,
—NR⁶(CO)NR⁷R⁸,
—NR⁶SO₂R⁹,
—COOR⁶,
—CONR⁶R⁷,
—COR⁶₁
—SO₂NR⁶R⁷,
—S(O)₀₋₂R⁹,
—O(CH₂)₁₋₁₀—COOR⁶,
—O(CH₂)₁₋₁₀CONR⁶R⁷,
-(lower alkylene)COOR⁶ and
—CH═CH—COOR⁶;
R⁶, R⁷ and R⁸ are independently selected from the group consisting of hydrogen, lower alkyl, aryl and aryl-substituted lower alkyl; and
R⁹ is lower alkyl, aryl or aryl-substituted lower alkyl.

11. The pharmaceutical composition of claim 10 wherein wherein R⁴ is 1-3 substituents independently selected from the group consisting of;
lower alkyl,
—OR⁶,
—O(CO)R⁶,
—O(CO)OR⁹,
—O(CH₂)₁₋₅OR⁶,
—O(CO)NR⁶R⁷,
—NR⁶R⁷,
—NR⁶(CO)R⁷,
—NR⁶(CO)OR⁹,
—NR⁶(CO)NR⁷R⁸,
—NR⁶SO₂R⁹,
—COOR⁶,
—CONR⁶R⁷,
—COR⁶,
—SO₂NR⁶R⁷,
—S(O)₀₋₂R⁹,
—O(CH₂)₁₋₁₀—COOR⁶,
—O(CH₂)₁₋₁₀CONR⁶R⁷,
-(lower alkylene)COOR⁶,
—CH═CH—COOR⁶,
—CF₃,
—CN,
—NO₂ and
halogen;

12. The pharmaceutical composition of claim 10 wherein R⁵ is 1-3 substituents independently selected from the group consisting of
—O(CO)R⁶,
—O(CO)OR⁹,
—O(CH₂)₁₋₅OR⁶,
—O(CO)NR⁶R⁷,
—NR⁶R⁷,
—NR⁶(CO)R⁷,
—NR⁶(CO)OR⁹,
—NR⁶(CO)NR⁷R⁸,
—NR⁶SO₂R⁹,
—COOR⁶,
—CONR⁶R⁷,
—COR⁶,
—SO₂NR⁶R⁷,
—S(O)₀₋₂R⁹,
—O(CH₂)₁₋₁₀—COOR⁶,
—O(CH₂)₁₋₁₀CONR⁶R⁷,
-(lower alkylene)COOR⁶ and
—CH═CH—COOR⁶;

13. The pharmaceutical composition of claim 10 wherein Ar1 is (4-R₄)— substituted phenyl.

14. The pharmaceutical composition of claim 10 wherein Ar2 is (4-R₄)— substituted phenyl.

15. The pharmaceutical composition of claim 10 wherein Ar3 is (4-R₅)— substituted phenyl.

16. The pharmaceutical composition of claim 1, wherein the sterol absorption inhibitor is represented by Formula (IX) 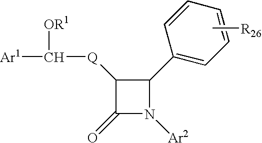
IX
or isomers thereof, or pharmaceutically acceptable salts or solvates of the compounds of Formula (I) or of the isomers thereof, or prodrugs of the compounds of Formula (IX) or of the isomers, salts or solvates thereof, wherein:
R²⁶ is selected from the group consisting of:
a) OH;
b) OCH₃;
c) fluorine and
d) chlorine.
R¹ is selected from the group consisting of
H, 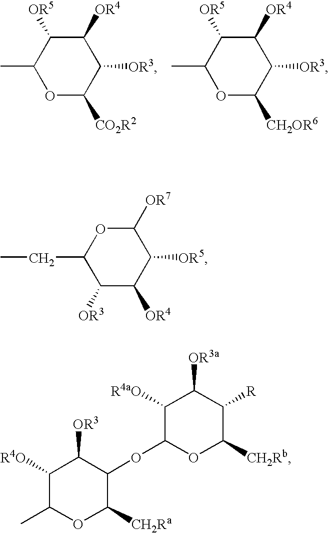
—SO₃H; natural and unnatural amino acids.
R, R^{a} and R^{b} are independently selected from the group consisting of H, —OH, halogeno, —NH₂, azido, (C₁-C₆)alkoxy(C₁-C₆)-alkoxy and —W—R³⁰;
W is independently selected from the group consisting of —NH—C(O)—, —O—C(O)—, —O—C(O)—N(R³¹)—, —NH—C(O)—N(R³¹)— and —O—C(S)—N(R³¹)—;
R² and R⁶ are independently selected from the group consisting of H, (C₁-C₆)alkyl, aryl and aryl(C₁-C₆)alkyl;
R³, R⁴, R⁵, R⁷, R₃ₐ and R^{4a} are independently selected from the group consisting of H, (C₁-C₆)alkyl, aryl(C₁-C₆)alkyl, —C(O)(C₁-C₆)alkyl and —C(O)aryl;
R³⁰ is independently selected form the group consisting of R³²-substituted T, R³²-substituted-T-(C₁-C₆)alkyl, R³²-substituted-(C₂-C₄)alkenyl, R³²-substituted-(C₁-C₆)alkyl, R³²-substituted-(C₃-C₇)cycloalkyl and R³²-substituted-(C₃-C₇)cycloalkyl(C₁-C₆)alkyl;
R³¹ is independently selected from the group consisting of H and (C₁-C₄)alkyl;
T is independently selected from the group consisting of phenyl, furyl, thienyl, pyrrolyl, oxazolyl, isoxazolyl, thiazolyl, iosthiazolyl, benzothiazolyl, thiadiazolyl, pyrazolyl, imidazolyl and pyridyl;
R³² is independently selected from 1-3 substituents independently selected from the group consisting of H, halogeno, (C₁-C₄)alkyl, —OH, phenoxy, —CF₃, —NO₂, (C₁-C₄)alkoxy, methylenedioxy, oxo, (C₁-C₄)alkylsulfanyl, (C₁-C₄)alkylsulfinyl, (C₁-C₄)alkylsulfonyl, —N(CH₃)₂, —C(O)—NH(C₁-C₄)alkyl, —C(O)—N((C₁-C₄)alkyl)₂, —C(O)—(C₁-C₄)alkyl, —C(O)—(C₁-C₄)alkoxy and pyrrolidinylcarbonyl; or R³² is a covalent bond and R³¹, the nitrogen to which it is attached and R³² form a pyrrolidinyl, piperidinyl, N-methyl-piperazinyl, indolinyl or morpholinyl group, or a (C₁-C₄)alkoxycarbonyl-substituted pyrrolidinyl, piperidinyl, N-methylpiperazinyl, indolinyl or morpholinyl group;
Ar¹ is aryl or R¹⁰-substituted aryl;
Ar² is aryl or R¹¹-substituted aryl;
Q is —(CH₂)_{q}—, wherein q is 2-6, or, with the 3-position ring carbon of the azetidinone,
forms the spiro group 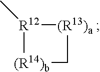
R₁₂ is 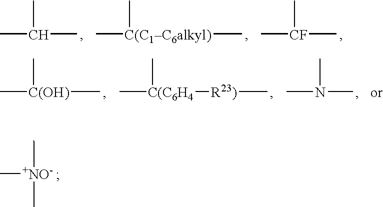
R₁₃ and R¹⁴ are independently selected from the group consisting of —CH₂—, —CH(C₁-C₆ alkyl)-, —C(di-(C₁-C₆) alkyl), —CH═CH— and —C(C₁-C₆ alkyl)=CH—; or R₁₂ together with an adjacent R¹³, or R¹² together with an adjacent R¹⁴, form a —CH═CH— or a —CH═C(C₁-C₆ alkyl)- group;
a and b are independently 0, 1, 2 or 3, provided both are not zero; provided that when R¹³ is —CH═CH— or —C(C₁-C₆ alkyl)=CH—, a is 1; provided that when R¹⁴ is —CH═CH— or —C(C₁-C₆ alkyl)=CH—, b is 1; provided that when a is 2 or 3, the R¹³′s can be the same or different; and provided that when b is 2 or 3, the R¹⁴′s can be the same or different;
R¹⁰ and R¹¹ are independently selected from the group consisting of 1-3 substituents independently selected from the group consisting of (C₁-C₆)alkyl, —OR¹⁹, —O(CO)R¹⁹, —O(CO)OR²¹, —O(CH₂)₁₋₅OR¹⁹, —O(CO)NR¹⁹R²⁰, —NR¹⁹R²⁰, —NR¹⁹(CO)R²⁰, —NR¹⁹(CO)OR²¹, —NR¹⁹(CO)NR²OR²⁵, —NR¹⁹SO₂R²¹, —COOR¹⁹, —CONR¹⁹R²⁰, —COR¹⁹, —SO₂NR¹⁹R²⁰, S(O)₀₋₂R²¹, —O(CH₂)₁₋₁₀—COOR¹⁹, —O(CH₂)₁₋₁₀CONR¹⁹R²⁰, —(C₁-C₆ alkylene)-COOR¹⁹, —CH═CH—COOR¹⁹, —CF₃, —CN, —NO₂ and halogen;
Ar¹ can also be pyridyl, isoxazolyl, furanyl, pyrrolyl, thienyl, imidazolyl, pyrazolyl, thiazolyl, pyrazinyl, pyrimidinyl or pyridazinyl;
R¹⁹ and R²⁰ are independently selected from the group consisting of H, (C₁-C₆)alkyl, aryl and aryl-substituted (C₁-C₆)alkyl;
R²¹ is (C₁-C₆)alkyl, aryl or R²⁴-substituted aryl;
R²² is H, (C₁-C₆)alkyl, aryl (C₁-C₆)alkyl, —C(O)R¹⁹ or —COOR¹⁹;
R²³ and R²⁴ are independently 1-3 groups independently selected from the group consisting of H, (C₁-C₆)alkyl, (C₁-C₆)alkoxy, —COOH, NO₂, —NR¹⁹R²⁰, —OH and halogeno; and
R²⁵ is H, —OH or (C₁-C₆)alkoxy.

17. The pharmaceutical composition of claim 16, wherein the sterol absorption inhibitor is represented by Formula (X): 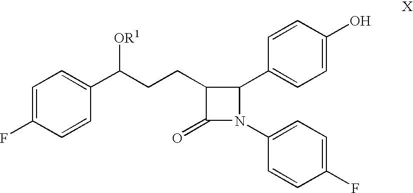
or isomers thereof, or pharmaceutically acceptable salts or solvates of the compounds of Formula (X) or of the isomers thereof, or prodrugs of the compounds of Formula (X) or of the isomers, salts or solvates thereof wherein:
R¹ is defined as above.

18. The pharmaceutical composition claim 16, wherein the sterol absorption inhibitor is represented by Formula (XI) 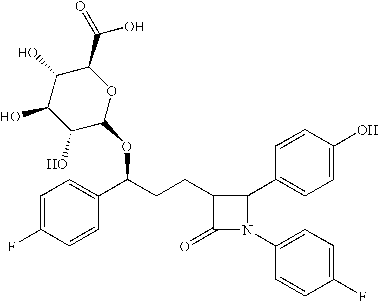
or isomers thereof, or pharmaceutically acceptable salts or solvates of the compounds of Formula (XI) or of the isomers thereof, or prodrugs of the compounds of Formula (XI) or of the isomers, salts or solvates thereof.

19. The pharmaceutical composition of claim 1, further comprising administering to the mammal in need of such treatment an effective amount of at least one lipid lowering agent in combination with the at least one sterol absorption inhibitor.

20. The method of claim 19, wherein the lipid lowering agent is a HMG-CoA reductase inhibitor.

21. The method of claim 20, wherein the HMG-CoA reductase inhibitor is selected from the group consisting of simvastatin, lovastatin, pravastatin, fluvastatin, atorvastatin, rosuvastatin, itavastatin and mixtures thereof.

22. The method of claim 21, wherein the HMG-CoA reductase inhibitor is simvastatin or atorvastatin.

23. A pharmaceutical composition comprising an effective amount of ezetimibe and an effective amount of at least one CETP inhibitor.

24. The pharmaceutical composition of claim 23, which further comprises as HMG-COA reductase inhibitor.

25. The pharmaceutical composition of claim 24, wherein the HMG-CoA reductase inhibitor is simvastatin or atorvastatin.

26. A method of treating or preventing sitosterolemia, hypercholesterolemia, hyperlipidemia, atherosclerosis, mixed dyslipidemia and vascular events prevention, which comprising administering the pharmaceutical composition of claim 1 a to a mammal in need of such treatment:
